(19) United States
(12) Patent Application Publication
(10) Pub. No.: US 2024/0282447 A1
(43) Pub. Date: Aug. 22, 2024

(54) A SYSTEM AND METHOD FOR MONITORING AND MANAGEMENT OF LIFESTYLE AND RELATED DISORDERS

(71) Applicant: Pramod Tripathi, Pune (IN)

(72) Inventor: Pramod Tripathi, Pune (IN)

(21) Appl. No.: 18/292,069

(22) PCT Filed: Jul. 1, 2022
(86) PCT No.: PCT/IN2022/050606

§ 371 Date: Jan. 25, 2024

(30) Foreign Application Priority Data
Jul. 28, 2021 (IN) 202141033982
Jun. 10, 2022 (IN) 202242033472

(51) Int. Cl. G16H 50/20; G16H 20/30; G16H 20/60

(52) U.S. Cl. CPC G16H 50/20; G16H 20/30; G16H 20/60

(57) ABSTRACT

A system and method having a multi-phased holistic approach for monitoring and management of lifestyle and related disorders is disclosed herein. The disclosed system uses a multiple phase approach to monitor and manage weight loss and reduce dependence of a subject on medication. The system provides a model approach based on input data achieved by protocols including a vegan diet, progressive exercises, meditations and practicing mindfulness.

### CROSS REFERENCE TO RELATED APPLICATIONS

This application is based on and derives the benefit of Indian Application IN202141033982 filed on Jul. 28, 2021, and IN202242033472 filed on Jun. 10, 2022, the contents of which are incorporated herein by reference in their entirety.

### TECHNICAL FIELD

The present invention relates to lifestyle, and related disorders, management systems and methods. It particularly relates to a system and method for achieving a healthy lifestyle by way of monitoring and managing factors affecting lifestyle, including optimizing insulin sensitivity, improving glycemic control, reducing dependence on anti-diabetic and other medications, achieving ideal BMI (Body mass index), improving fitness levels, reducing stress, improving positivity, and proper medical and supplement management.

### BACKGROUND

Lifestyle management is crucial in prevention and management of diseases. Unhealthy lifestyle is considered a root cause for lifestyle disorders such as diabetes, obesity, hypertension, cardiovascular diseases (CVDs), etc. Diabetes has become a major health concern necessitating global attention. According to International Diabetes Federation, the total number of adults having Diabetes is expected to rise from 463 million in 2019 to around 700 million by the year 2045. In view of increase in the worldwide incidence of diabetes, the United Nations and the World Health Organization (WHO) are now emphasizing on diabetes as a major health concern requiring immediate consideration. Due to increasing levels of obesity, sedentary lifestyle and poor diet, diabetes is on the rise with an estimated 77 million diabetics in India. The long-term effects of diabetes include macrovascular and microvascular complications like myocardial infarctions, lower extremity amputations, end stage renal disease, coronary artery disease (CAD), stroke, and reduction in life expectancy.

While there is no cure for this condition, various therapeutic approaches have been explored to alleviate its effects. The first and common line of treatment includes evaluation of sugar levels followed by the administration of hypoglycemic drugs and subsequently insulin therapy. However, owing to the progressive nature of the disease, therapeutic regimen gradually become less efficacious requiring a change from time to time.

In addition to medication, lifestyle modifications focusing on balanced diet, regular physical exercise, weight loss, and stress management have been observed to contribute towards better long-term management of blood glucose levels. Role of diet and exercise in the management of diseases such as diabetes and CVDs has been well established. Various ayurvedic intervention and yogic practices have been shown to be effective in reducing blood glucose level in subjects with type II diabetes mellitus.

Lifestyle has a significant influence on the physical and mental health of a being. Various factors that influence lifestyle include diet, body mass index (BMI), exercise, sleep, medication abuse, etc. Efficient monitoring and management of lifestyle is crucial to achieving a well-functioning body and mind. Therefore, there exists a need for an integrated approach for a holistic lifestyle management method and system which is efficacious, stunts progression of the diseases and reduces dependency on medications.

### OBJECTS OF THE DISCLOSED EMBODIMENTS

The principal object of the embodiments disclosed herein is to provide a system for monitoring and holistic management of lifestyle.

A second object of the embodiments disclosed herein is to provide a system for monitoring and holistic management of disorders associated with unhealthy lifestyle.

An object of the embodiments disclosed herein is to provide a holistic method for reducing a subject's dependence on medications.

Another object of the embodiments disclosed herein is to provide system for improving glycemic control in diabetic and prediabetic individuals.

Another object of the embodiment disclosed herein is to provide a system and method for holistic management and reversal of lifestyle associated disorders such as obesity, high blood pressure, high cholesterol, polycystic ovarian disease (PCOD), hypothyroidism, Coronary artery disease (CAD), stroke, etc.)

Another object of the embodiments disclosed herein is to provide a holistic method for improving the levels of important biomarkers for metabolic syndrome including but not limited to serum creatinine level, uric acid level and urine microalbumin level.

Another object of the embodiments disclosed herein is to provide a method for achieving and maintaining a healthy BMI by adjusting the diet and exercises based on subject's BMI.

Another object of the embodiments disclosed herein is to provide a system to monitor and manage a subject's health progress.

These and other objects of the embodiments herein will be better appreciated and understood when considered in conjunction with the following description and the accompanying tables and figures. Many changes and modifications may be made within the scope of the embodiments herein without departing from the spirit thereof, and the embodiments herein include such modifications.

### BRIEF DESCRIPTION OF DRAWINGS

The embodiments are illustrated in the accompanying drawings. The embodiments herein will be better understood from the following description with reference to the drawings, in which:

FIGS. 1(a) and 1(b) show exemplary blocks of the system 100, according to embodiments herein.

FIG. 2 is an example block diagram depicting various components of the subject monitoring device 108 to generate the results of the subject based on the input data, according to embodiments as disclosed herein.

FIGS. 3(a) and 3(b) are flowcharts depicting method (200) to monitor and/or manage the health progress of the subject, according to embodiments as disclosed herein.

FIG. 4 is a graphical representation of the age distribution among the 470 number of Type 2 diabetic/prediabetic participants in Group 1, undergoing the controlled intervention program, according to the embodiments herein.

FIG. 5 graphically depicts the percentage of Body Mass Index (BMI) of the Type 2 diabetic/prediabetic participants in Group 1, both before and after undergoing the controlled intervention program, according to the embodiments herein.

FIG. 6 is a depiction of the absolute change in weight in kilograms, of the Type 2 diabetic/prediabetic participants in Group 1, undergoing the controlled intervention program, according to the embodiments herein.

FIG. 7 is a graphical representation of the number of individuals in Group 1 who are no longer on medication to manage or treat various disorders like hypertension and diabetes at the end of the controlled intervention program, according to the embodiments herein.

FIG. 8 is a graphical representation depicting the percentage of glycated hemoglobin (HbA1c) of the Type 2 diabetic/prediabetic participants in Group 1, both before and after undergoing the controlled intervention program, according to the embodiments herein.

FIGS. 9(a) and 9(b) are representations depicting the percentage of 470 individuals in Group 1 with normal and high levels of cholesterol at the beginning and at the end, respectively, of the controlled intervention program, according to the embodiments herein.

FIGS. 10(a) and 10(b) are representations depicting the percentage of 470 individuals in Group 1 with normal and high levels of Thyroid Stimulating Hormone (TSH) at the beginning and at the end, respectively, of the controlled intervention program, according to the embodiments herein.

FIGS. 11(a) and 11(b) are representations depicting the percentage of individuals in Group 1 with normal and high levels of creatinine in women at the beginning and at the end, respectively, of the controlled intervention program, according to the embodiments herein

FIGS. 12(a) and 12(b) are representations depicting the percentage of individuals in Group 1 with normal and high levels of creatinine in men at the beginning and at the end, respectively, of the controlled intervention program, according to the embodiments herein.

FIG. 13 is a graphical representation of the age distribution among the 643 number of Type 2 diabetic/prediabetic participants in Group 2, undergoing the controlled intervention program, according to the embodiments herein.

FIG. 14 is a representation depicting the percentage of Body Mass Index (BMI) of the Type 2 diabetic/prediabetic participants in Group 2, both before and after undergoing the controlled intervention program, according to the embodiments herein.

FIG. 15 is a representation depicting the absolute change in weight in kilograms, of the Type 2 diabetic/prediabetic participants in Group 2, undergoing the controlled intervention program, according to the embodiments herein.

FIG. 16 is a graphical representation of the number of individuals in Group 2 who are no longer on medication to manage or treat various disorders like hypertension and diabetes at the end of the controlled intervention program, according to the embodiments herein.

FIG. 17 graphically depicts the percentage of glycated hemoglobin (HbA1c) of the Type 2 diabetic/prediabetic participants in Group 2, both before and after undergoing the controlled intervention program, according to the embodiments herein.

FIGS. 18(a) and 18(b) represent the percentage of 643 individuals in Group 2 with normal and high levels of cholesterol at the beginning and at the end, respectively, of the controlled intervention program, according to the embodiments herein.

FIGS. 19(a) and 19(b) represent the percentage of 643 individuals in Group 2 with normal and high levels of Thyroid Stimulating Hormone (TSH) at the beginning and at the end, respectively, of the controlled intervention program, according to the embodiments herein.

FIGS. 20(a), 20(b) and 20(c) represent the percentage of individuals in Group 2 with normal and high levels of creatinine in both women and men, at the beginning and at the end, respectively, of the controlled intervention program, according to the embodiments herein.

FIG. 21 is a graphical representation of the age distribution among the 711 number of Type 2 diabetic/prediabetic participants in Group 3, undergoing the controlled intervention program, according to the embodiments herein.

FIG. 22 is a representation depicting the percentage of Body Mass Index (BMI) of the Type 2 diabetic/prediabetic participants in Group 3, both before and after undergoing the controlled intervention program, according to the embodiments herein.

FIG. 23 is a representation depicting the absolute change in weight in kilograms, of the Type 2 diabetic/prediabetic participants in Group 3, undergoing the controlled intervention program, according to the embodiments herein.

FIG. 24 is a graphical representation of the number of individuals in Group 3 who are no longer on medication to manage or treat various disorders like hypertension and diabetes at the end of the controlled intervention program, according to the embodiments herein.

FIG. 25 is a representation depicting the percentage of glycated hemoglobin (HbA1c) of the Type 2 diabetic/prediabetic participants in Group 3, both before and after undergoing the controlled intervention program, according to the embodiments herein.

FIGS. 26(a) and 26(b) are graphical representations depicting the percentage of 711 individuals in Group 3 with normal and high levels of cholesterol at the beginning and at the end, respectively, of the controlled intervention program, according to the embodiments herein.

FIGS. 27(a) and 27(b) are graphical representations depicting the percentage of 711 individuals in Group 3 with normal and high levels of Thyroid Stimulating Hormone (TSH) at the beginning and at the end, respectively, of the controlled intervention program, according to the embodiments herein.

FIGS. 28(a), 28(b) and 28(c) are graphical representations depicting the percentage of individuals in Group 3 with normal and high levels of creatinine in both women and men, at the beginning and at the end, respectively, of the controlled intervention program, according to the embodiments herein.

FIG. 29 is a graphical representation depicting the age distribution among the 376 number of Type 2 diabetic/prediabetic participants in Group 4, undergoing the controlled intervention program, according to the embodiments herein.

FIG. 30 is a graphical representation depicting the percentage of Body Mass Index (BMI) of the Type 2 diabetic/prediabetic participants in Group 4, both before and after undergoing the controlled intervention program, according to the embodiments herein.

FIG. 31 is a graphical representation depicting the absolute change in weight in kilograms, of the Type 2 diabetic/prediabetic participants in Group 4, undergoing the controlled intervention program, according to the embodiments herein.

FIG. 32 is a graphical representation depicting the number of individuals in Group 4 who are no longer on medication to manage or treat various disorders like hypertension and diabetes at the end of the controlled intervention program, according to the embodiments herein.

FIG. 33 is a graphical representation depicting the percentage of glycated hemoglobin (HbA1c) of the Type 2 diabetic/prediabetic participants in Group 4, both before and after undergoing the controlled intervention program, according to the embodiments herein.

FIGS. 34(a) and 34(b) are graphical representations depicting the percentage of 376 individuals in Group 4 with normal and high levels of cholesterol at the beginning and at the end, respectively, of the controlled intervention program, according to the embodiments herein.

FIGS. 35(a) and 35(b) are graphical representations depicting the percentage of 376 individuals in Group 4 with normal and high levels of Thyroid Stimulating Hormone (TSH) at the beginning and at the end, respectively, of the controlled intervention program, according to the embodiments herein.

FIGS. 36(a) and 36(b) are graphical representations depicting the percentage of individuals in Group 4 with normal and high levels of creatinine in women at the beginning and at the end, respectively, of the controlled intervention program, according to the embodiments herein

FIGS. 37(a) and 37(b) are a graphical representation depicting the percentage of individuals in Group 4 with normal and high levels of creatinine in men at the beginning and at the end, respectively, of the controlled intervention program, according to the embodiments herein.

FIG. 38 is a graphical representation depicting the age distribution among the 470 number of Type 2 diabetic/prediabetic participants in Group 5, undergoing the controlled intervention program, according to the embodiments herein.

FIG. 39 is a graphical representation depicting the percentage of Body Mass Index (BMI) of the Type 2 diabetic/prediabetic participants in Group 5, both before and after undergoing the controlled intervention program, according to the embodiments herein.

FIG. 40 is a depiction of the absolute change in weight in kilograms, of the Type 2 diabetic/prediabetic participants in Group 5, undergoing the controlled intervention program, according to the embodiments herein.

FIG. 41 is a graphical representation depicting the number of individuals in Group 5 who are no longer on medication to manage or treat various disorders like hypertension and diabetes at the end of the controlled intervention program, according to the embodiments herein.

FIG. 42 is a graphical representation depicting the percentage of glycated hemoglobin (HbA1c) of the Type 2 diabetic/prediabetic participants in Group 5, both before and after undergoing the controlled intervention program, according to the embodiments herein.

FIGS. 43(a) and 43(b) are graphical representations depicting the percentage of 470 individuals in Group 5 with normal and high levels of cholesterol at the beginning and at the end, respectively, of the controlled intervention program, according to the embodiments herein.

FIGS. 44(a) and 44(b) are graphical representations depicting the percentage of 470 individuals in Group 5 with normal and high levels of Thyroid Stimulating Hormone (TSH) at the beginning and at the end, respectively, of the controlled intervention program, according to the embodiments herein.

FIGS. 45(a) and 45(b) are graphical representations depicting the percentage of individuals in Group 5 with normal and high levels of creatinine in women at the beginning and at the end, respectively, of the controlled intervention program, according to the embodiments herein

FIGS. 46(a) and 46(b) are graphical representations depicting the percentage of individuals in Group 5 with normal and high levels of creatinine in men at the beginning and at the end, respectively, of the controlled intervention program, according to the embodiments herein.

FIG. 47 is a graphical representation depicting the age distribution among the 602 number of Type 2 diabetic/prediabetic participants in Group 6, undergoing the controlled intervention program, according to the embodiments herein.

FIG. 48 is a graphical representation depicting the percentage of Body Mass Index (BMI) of the Type 2 diabetic/prediabetic participants in Group 6, both before and after undergoing the controlled intervention program, according to the embodiments herein.

FIG. 49 is a graphical representation depicting the absolute change in weight in kilograms, of the Type 2 diabetic/prediabetic participants in Group 6, undergoing the controlled intervention program, according to the embodiments herein.

FIG. 50 is a graphical representation of the number of individuals in Group 6 who are no longer on medication to manage or treat various disorders like hypertension and diabetes at the end of the controlled intervention program, according to the embodiments herein.

FIG. 51 is a graphical representation depicting the percentage of glycated hemoglobin (HbA1c) of the Type 2 diabetic/prediabetic participants in Group 6, both before and after undergoing the controlled intervention program, according to the embodiments herein.

FIGS. 52(a) and 52(b) are graphical representations depicting the percentage of 602 individuals in Group 6 with normal and high levels of cholesterol at the beginning and at the end, respectively, of the controlled intervention program, according to the embodiments herein.

FIGS. 53(a) and 53(b) are graphical representations depicting the percentage of 602 individuals in Group 6 with normal and high levels of Thyroid Stimulating Hormone (TSH) at the beginning and at the end, respectively, of the controlled intervention program, according to the embodiments herein.

FIGS. 54(a) and 54(b) are graphical representations depicting the percentage of individuals in Group 6 with normal and high levels of creatinine in women at the beginning and at the end, respectively, of the controlled intervention program, according to the embodiments herein

FIGS. 55(a) and 55(b) are graphical representations depicting the percentage of individuals in Group 6 with normal and high levels is a graphical of creatinine in men at the beginning and at the end, respectively, of the controlled intervention program, according to the embodiments herein.

FIG. 56 is a representation depicting the age distribution among the 654 number of Type 2 diabetic/prediabetic participants in Group 7, undergoing the controlled intervention program, according to the embodiments herein.

FIG. 57 is a representation depicting the percentage of Body Mass Index (BMI) of the Type 2 diabetic/prediabetic participants in Group 7, both before and after undergoing the controlled intervention program, according to the embodiments herein.

FIG. 58 is representation depicting the absolute change in weight in kilograms, of the Type 2 diabetic/prediabetic participants in Group 7, undergoing the controlled intervention program, according to the embodiments herein.

FIG. 59 is a graphical representation depicting the number of individuals in 5 Group 7 who are no longer on medication to manage or treat various disorders like hypertension and diabetes at the end of the controlled intervention program, according to the embodiments herein.

FIG. 60 is a graphical representation depicting the percentage of glycated hemoglobin (HbA1c) of the Type 2 diabetic/prediabetic participants in Group 7, both before and after undergoing the controlled intervention program, according to the embodiments herein.

FIGS. 61(a) and 61(b) are graphical representations depicting the percentage of 654 individuals in Group 7 with normal and high levels of cholesterol at the beginning and at the end, respectively, of the controlled intervention program, according to the embodiments herein.

FIGS. 62(a) and 62(b) are graphical representations depicting the percentage of 654 individuals in Group 7 with normal and high levels of Thyroid Stimulating Hormone (TSH) at the beginning and at the end, respectively, of the controlled intervention program, according to the embodiments herein.

FIGS. 63(a) and 63(b) are graphical representations depicting the percentage of individuals in Group 7 with normal and high levels of creatinine in women at the beginning and at the end, respectively, of the controlled intervention program, according to the embodiments herein

FIGS. 64(a) and 64(b) are graphical representations depicting the percentage of individuals in Group 7 with normal and high levels of creatinine in men at the beginning and at the end, respectively, of the controlled intervention program, according to the embodiments herein.

### DETAILED DESCRIPTION

The embodiments herein and the various features and advantageous details thereof are explained more fully and detailed in the following description. Descriptions of well-known components, ingredients, and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The embodiments disclosed herein, provide a system and method having a holistic approach for monitoring and management of lifestyle. Embodiments herein provide a system for monitoring and holistic management of lifestyle and associated disorders such as diabetes, obesity, hypertension, cardiovascular diseases (CVDs), etc. Embodiments herein include a system and method for improving health parameters such as blood glucose level, serum creatinine in diabetic and prediabetic subjects. The method, as disclosed herein, is observed to alleviate, and even reverse associated metabolic complications. Further, the embodiments herein, are also capable of significantly reducing a subject's requirement for medication. Accordingly, a method for reducing the subject's dependence on medication, eg: antidiabetic medication, is disclosed herein. Disclosed herein are embodiments of a method for the prevention, management, and reversal of complications associated with unhealthy lifestyle. Also disclosed is a system for monitoring and managing subject's health progress.

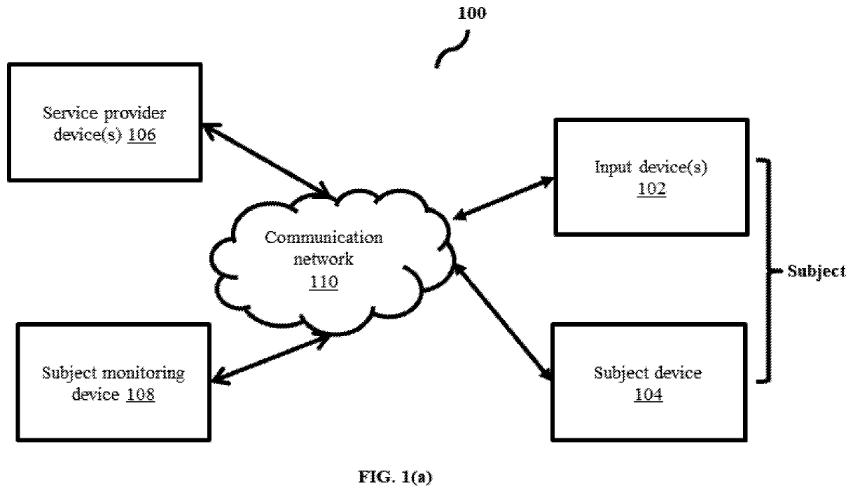

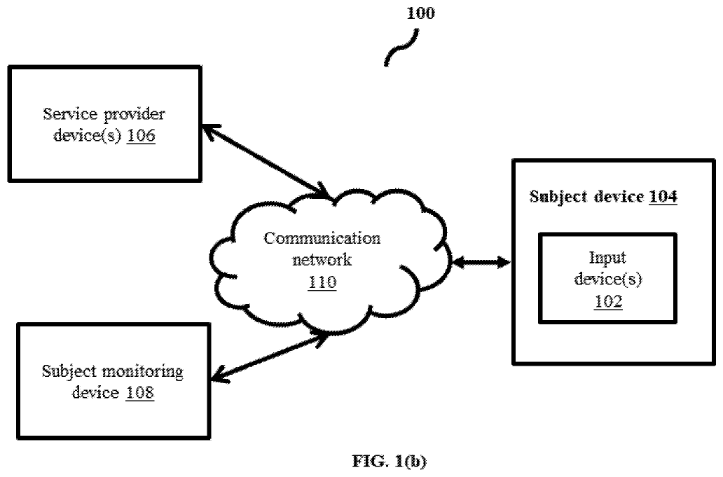

Referring now to FIGS. 1(a) and 1(b) which depict a system 100 to monitor and/or manage the health progress of the subject, according to embodiments as disclosed herein. The system provides a holistic approach on monitoring and management of lifestyle and lifestyle associated disorders.

The system 100 includes one or more input devices 102, a subject device 104, a service provider device 106, and a subject monitoring device 108. The input device(s) 102, the subject device 104, the service provider device 106, and the subject monitoring device 108 may be connected with each other using a communication network/carrier network 110. The communication network 110 may include at least one of, but is not limited to, a wired network, a value-added network, a wireless network, a satellite network, or a combination thereof. Examples of the wired network may be, but are not limited to, a Local Area Network (LAN), a Wide Area Network (WAN), an Ethernet, and so on. Examples of the wireless network may be, but are not limited to, a cellular network, a wireless LAN (Wi-Fi), Bluetooth, Bluetooth low energy, Zigbee, Wi-Fi Direct (WFD), Ultra-wideband (UWB), infrared data association (IrDA), near field communication (NFC), and so on. In another example, the input device(s) 102, the subject device 104, the service provider device 106, and the subject monitoring device 108 may be connected with each other directly (for example: via a direct communication, via an access point, and so on). In another example, the input device(s) 102, the subject device 104, the service provider device 106, and the subject monitoring device 108 may be connected with each other via a relay, a hub, and a gateway. It is understood that the input device(s) 102, the subject device 104, the service provider device 106, and the subject monitoring device 108 may be connected to each other in any of various manners (including those described above) and may be connected to each other in two or more of various manners (including those described above) at the same time.

The input device(s) 102 referred herein may be a device associated with the subject, which is configured to capture the input data of the subject. In an example, the input device 102 of the subject may be communicatively connected to the subject device 104 of the respective subject through the communication network 110, as depicted in FIG. 1(a). In another example, the input device 102 may be integrated with the subject device 104, as depicted in FIG. 1(b).

Examples of the input device 102 may be, but are not limited to, one or more glucometers for analyzing blood glucose levels, fitness meters, trackers, diet trackers or meters (for example: fitbit), nutrition trackers or managers, calorie trackers, or any other device capable of capturing the input data of the subject. A function of each of the exemplary input devices 102 referred herein may be intuitively inferred by one of ordinary skill in the art based on its name or type, and thus, its detailed description is omitted.

Examples of the input data may be, but are not limited to, subject's medication requirement, weight, fitness levels, stress levels, fasting blood glucose level (mg/dl), post prandial glucose level, HbA1C level (%), BMI (Body Mass Index) (kg/m2), and subject's waist circumference (cm), subject's lifestyle related parameters, and so on. Examples of the lifestyle related parameters may be, but are not limited to, subject's age, height, other medical conditions, blood pressure, blood group, genetic factors, and so on. In an embodiment, the input data are initial levels of markers, which may be used to assess improvement in the health conditions of the subject.

The one or more input devices 102 provides the captured input data of the subject to the subject monitoring device 108.

The subject device 104 referred herein may be a device being used by the subject to receive the results from the subject monitoring device 108, depending on the input data of the subject. The service provider device 106 referred herein may be a device being used by a service provider to receive the results corresponding to the subject from the subject monitoring device 108, depending on the input data of the subject. In an example herein, the service provider may include at least one of, but is not limited to, a healthcare professional, a doctor, and so on. Examples of the subject device 104 and the service provider device 106 may be, but are not limited to, a smart phone, a mobile phone, a video phone, a computer, a tablet personal computer (PC), a netbook computer, a laptop, a wearable device, a personal digital assistant (PDA), a fax machine, a workstation, a server, an Internet of Things (IoT) device, and so on.

The subject monitoring device 108 referred herein may be least one of, but is not limited to, a cloud computing device (can be a part of a public cloud or a private cloud), a server, a database, a computing device, and so on. The server may be at least one of a standalone server, a server on a cloud, or the like. The computing device may be, but is not limited to, a personal computer, a notebook, a tablet, desktop computer, a laptop, a handheld device, a mobile device, and so on. Also, the subject monitoring device 108 may be at least one of, a microcontroller, a processor, a System on Chip (SoC), an integrated chip (IC), a microprocessor based programmable consumer electronic device, and so on.

The subject monitoring device 108 may be configured to generate the results for the subject, by processing the input data of the subject received from the associated one or more input devices 102. Examples of the results may be, but are not limited to, subject's medication requirement, weight, fitness levels, stress levels, fasting blood glucose level (mg/dl), post prandial glucose level, HbA1C level (%), BMI (Body Mass Index) (kg/m2), subject's waist circumference (cm), and so on, taken or evaluated after the treatment or a phase, and so on.

The subject monitoring device 108 may also be configured to monitor the markers such as blood glucose, HbA1c levels, medications, physical activity, weight loss, of the subject. Thus, the results may also include the markers evaluated at any point after the treatment is started for the subject. The results may be used for lifestyle modifications or determining subject's health conditions.

The subject monitoring device 108 may also be configured to provide a prognosis based on the health conditions of the subject. The subject monitoring device 108 provides the prognosis by evaluating the difference in the input data and the results of the subject.

The subject monitoring device 108 may also be configured to suggest a diet and exercise protocol based on the health conditions of the subject. In an example, the subject monitoring device 108 suggests the diet and exercise protocol with the support of at least one of, but is not limited to, the healthcare professional, the doctor, and so on. In another example, the subject monitoring device 108 suggests the diet and exercise protocol without the support of at least one of, but is not limited to, the healthcare professional, the doctor, and so on.

The subject monitoring device 108 provides at least one of, the results, the prognosis, the diet, and exercise protocol, and so on, corresponding to the subject to at least one of, the subject device 104/subject, the service provider device 106/service provider, or the like through the communication network 110. In an example herein, the subject monitoring device 108 provides at least one of, the results, the prognosis, the diet and exercise protocol, and so on, corresponding to the subject as the message (written message, visual message and audio message) to the subject, the service provider, or the like.

The subject monitoring device 108 may also be configured to process and monitor the results of the subjects based on the input data, in a closed loop control operation and to display the results to an authorized person.

The subject monitoring device 108 may also be configured to schedule group sessions, provide doctor's support, and provide alert, reminders, notification and/or alarms to the subject/healthcare provider.

FIGS. 1(a) and 1(b) show exemplary blocks of the system 100, but it is to be understood that other embodiments are not limited thereon. In other embodiments, the system 100 may include less or more number of blocks. Further, the labels or names of the blocks are used only for illustrative purpose and does not limit the scope of the embodiments herein. One or more blocks can be combined together to perform same or substantially similar function in the system 100.

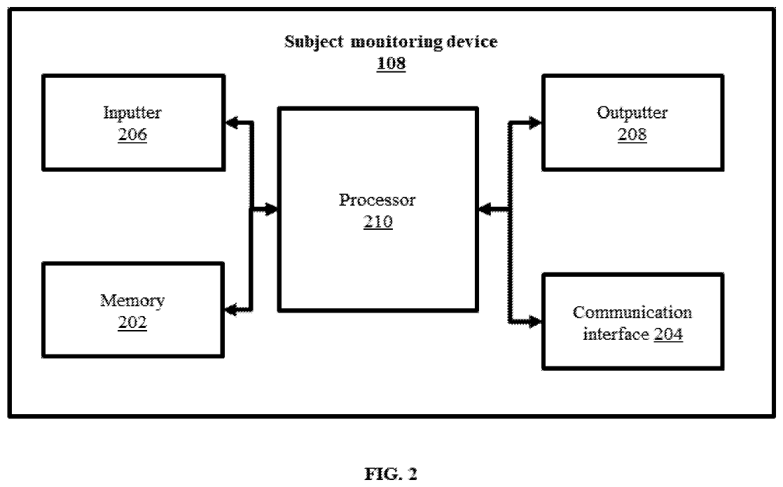

FIG. 2 is an example block diagram depicting various components of the subject monitoring device 108 to generate the results of the subject based on the input data, according to embodiments as disclosed herein. The subject monitoring device 108 includes a memory 202, a communication interface 204, an inputter 206, an outputter 208, and a processor 210.

The memory 202 may store at least one of, but is not limited to, the input data of the subject, the generated results corresponding to the input data of the subject, the prognosis provided based on the health conditions of the subject, the diet and exercise protocol provided based on the health conditions of the subject, and so on. The memory 202 referred herein may include at least one type of storage medium, from among a flash memory type storage medium, a hard disk type storage medium, a multi-media card micro type storage medium, a card type memory (for example, an SD or an XD memory), random-access memory (RAM), static RAM (SRAM), read-only memory (ROM), electrically erasable programmable ROM (EEPROM), programmable ROM (PROM), a magnetic memory, a magnetic disk, or an optical disk.

The communication interface 204 may be configured to enable the subject monitoring device 108 to communicate with the input devices 102, the subject device 104, the service provider device 106, or the like, using an interface supported by the communication network 110. Examples of the interface may be, but are not limited to, a wired interface, a wireless interface, a wired interface, or any structure supporting communications over a wired or wireless connection.

The inputter 206 may be configured to receive the input data of the subject from the associated one or more input devices 102.

The outputter 208 may be configured to enable the authorized person to interact with the subject monitoring device 108. The outputter 208 may also be configured to receive the results corresponding to the subject from the processor 210 and provide the results to at least one of, the authorized person, the subject, the service provider, and so on. The outputter 208 provides the results corresponding to the subject as the message (written message, visual message and audio message) to the subject, the service provider, or the like through the communication interface 204. The outputter 208 may include at least one of, for example, but is not limited to, a sound outputter/voice assistant module, a display, a vibration motor, a User Interface (UI) module, a light emitting device, and so on, to provide the results corresponding to the subject to the authorized person. The UI module may provide a specialized UI or graphics user interface (GUI), or the like, synchronized to the subject monitoring device 108, according to the applications.

The processor 210 referred herein may include at least one of, a single processer, a plurality of processors, multiple homogeneous or heterogeneous cores, multiple Central Processing Units (CPUs) of different kinds, microcontrollers, special media, and other accelerators. The processor 210, according to embodiments herein, is configured such that it is capable of computing the subject's input data to provide the results based on the method according to embodiments herein.

The processor 210 may be configured to generate the results corresponding to the subject by processing the input data of the subject. The results may be used to monitor and/or manage the health progress of the subject. The processor 210 may provide the results corresponding to the subject to the outputter 208.

The processor 210 may also be configured to provide the prognosis and the diet and exercise protocol based on the health conditions of the subject, to the subject, the service provider, or the like through the outputter 208.

The processor 210 may also be configured to process and monitor the results of the subject based on the input data in the closed loop control operation.

FIG. 2 shows exemplary blocks of the subject monitoring device 108, but it is to be understood that other embodiments are not limited thereon. In other embodiments, the subject monitoring device 108 may include less or more number of blocks. Further, the labels or names of the blocks are used only for illustrative purpose and does not limit the scope of the embodiments herein. One or more blocks can be combined together to perform same or substantially similar function in the subject monitoring device 108.

In an embodiment, the system (100) comprises at least one input device (102) associated with the subject, the input device (102) is configured to: capture input data of the subject, wherein the input data is received by subjecting said subject to, an alkalizing diet, an exercise protocol comprising circulation and activation exercises, and determining improvement in subject's health condition; a cleansing diet, or SNS meal plan for subject requiring additional weight loss, an exercise protocol comprising yoga and strengthening exercises, and determining improvement in subject's health condition; a cleansing diet or a muscle building diet, an exercise protocol comprising of yoga and strengthening exercises or sampling and athletic specialization exercises, a meditation protocol for release of stress, and determining improvement in subject's health condition; a muscle building diet, an exercise protocol comprising sampling and specialization exercises; and determining improvement in subject's health condition; and a specific diet and exercise chart to sustain reversal and phase 4 athletic identity. Further, the system (100) includes the subject monitoring device (108) coupled to the at least one input device (102), where the subject monitoring device (108) is configured to: generate results for the subject by processing the input data of the subject captured by the at least one input device (102); process and monitor the generated results of the subject based on the input data in a closed loop control operation for management of lifestyle and associated disorders in the subject; and provide the results of the subject as a message (written message, visual message and audio message) to at least one of, the subject, a healthcare professional, and a doctor.

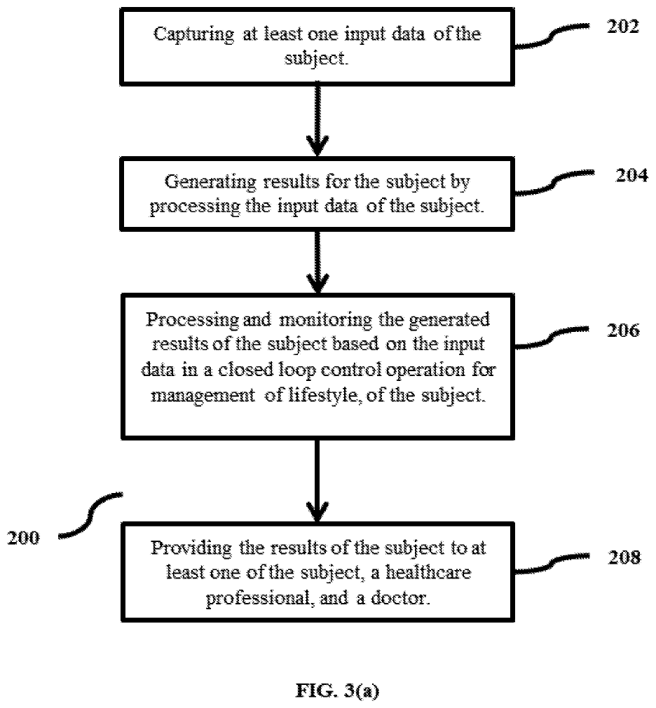

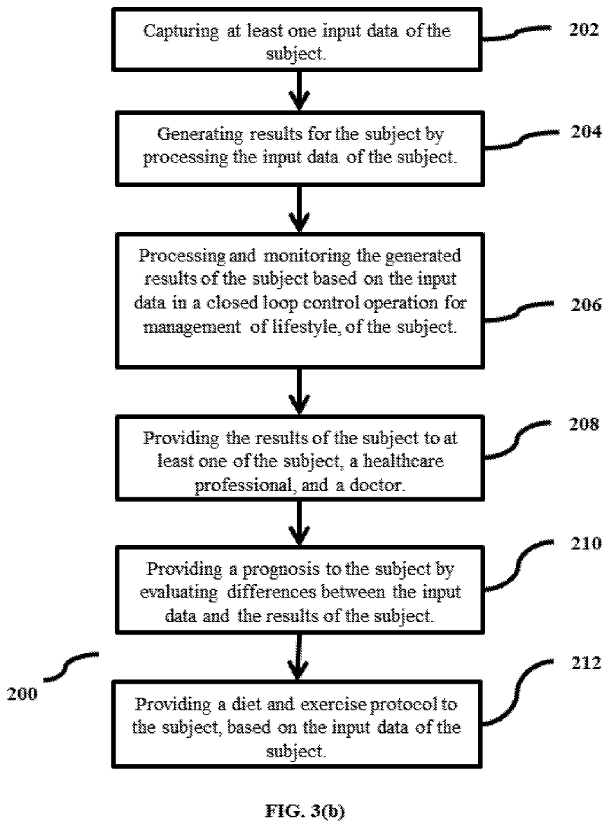

Embodiments herein include a method for monitoring and/or managing lifestyle, lifestyle related disorders or health progress of the subject. Referring now to FIGS. 3(a) and 3(b) which depict a method (200) to monitor and/or manage the health progress of the subject, according to embodiments as disclosed herein. The method provides a holistic approach on monitoring and management of lifestyle and associated disorders. The method (200) includes capturing (202) at least one input data of the subject; generating (204) results for the subject by processing the input data of the subject; processing and monitoring (206) the generated results of the subject based on the input data in a closed loop control operation for management of lifestyle of the subject; and providing (208) the results of the subject to at least one of, the subject, a healthcare professional, and a doctor. In an embodiment, the method (200) is achieved using the system (100).

In an embodiment, the method includes capturing (202) by at least one input device (102) associated with the subject, at least one input data of the subject; generating (204), by a subject monitoring device (108), results for the subject by processing the input data of the subject captured by the at least one input device (102); processing and monitoring (206), by the subject monitoring device (108), the generated results of the subject based on the input data in a closed loop control operation for management of lifestyle, of the subject; and providing (208), by the subject monitoring device (108), the results of the subject to at least one of, the subject, a healthcare professional, and a doctor.

The input data capturing (202) of at least one input data, according to embodiments herein, further includes receiving the at least one input data by subjecting the subject to: an alkalizing diet, an exercise protocol comprising circulation and activation exercises, and determining improvement in subject's health condition; a cleansing diet, or SNS meal plan for subject requiring additional weight loss, an exercise protocol comprising yoga and strengthening exercises, and determining improvement in subject's health condition; cleansing diet or a muscle building diet, an exercise protocol comprising of yoga and strengthening exercises or sampling and athletic specialization exercises, a meditation protocol for release of stress, and determining improvement in subject's health condition; the muscle building diet, an exercise protocol comprising sampling and specialization exercises, and determining improvement in subject's health condition; and a specific diet and exercise chart to sustain reversal and phase 4 athletic identity.

In another embodiment, the method comprises capturing (202) at least one input data of the subject; generating (204) results for the subject by processing the input data of the subject; processing and monitoring (206) the generated results of the subject based on the input data in a closed loop control operation for management of lifestyle of the subject; providing (208) the results of the subject to at least one of, the subject, a healthcare professional, and a doctor; providing (210), by the subject monitoring device (108), a prognosis to the subject by evaluating differences between the input data and the results of the subject; and providing (212), by the subject monitoring device (108), a diet and exercise protocol to the subject, based on the input data of the subject.

The term “lifestyle related disorders” or “lifestyle associated disorders”

Embodiments herein further include a method for prevention, treatment, management, and/or reversal of Diabetes and related lifestyle disorders in a subject. In an embodiment, the method comprises subjecting said subject to an alkalizing diet, an exercise protocol comprising circulation and activation exercises, and determining improvement in subject's condition; a cleansing diet, or SNS meal plan for subject requiring additional weight loss, an exercise protocol comprising yoga and strengthening exercises, and determining improvement in subject's condition; a cleansing diet or a muscle building diet, an exercise protocol comprising of yoga and strengthening exercises or sampling and athletic specialization exercises, a meditation protocol for release of stress, and determining improvement in subject's condition; a muscle building diet, an exercise protocol comprising sampling and specialization exercises; and determining improvement in subject's condition; and a specific diet and exercise chart to sustain reversal and phase 4 athletic identity. In an embodiment, the diet is a vegan diet which is free of milk and milk product, wherein said vegan diet comprises a green smoothie comprising at least one green leafy vegetable selected from a group consisting of spinach, kale, lettuce and green sorrel; at least one fruit selected from a group consisting of apple, pear, guava, banana and papaya; leaves of at least two herbs selected from a group consisting of Murraya koenigii, Piper betle, Ocimum tenuiflorum, basil, and mint leaves; protein rich breakfast comprising sprouts at a portion size of 25%, salads at a portion size of 25% and cooked Lentil-based food at a portion size of 50%, of said breakfast; and a principle meal comprising 25% of each of raw salad, cooked vegetable, grain and lentil-based food.

In an embodiment, each diet is a vegan diet which is free of milk and milk product. In an embodiment, the vegan diet comprises a green smoothie comprising at least one green leafy vegetable selected from a group consisting of spinach, kale, lettuce and green sorrel; at least one fruit selected from a group consisting of apple, pear, guava, banana and papaya; leaves of at least two herbs selected from a group consisting of Murraya koenigii, Piper betle, Ocimum tenuiflorum, basil, and mint leaves; protein rich breakfast comprising sprouts at a portion size of 25%, salads at a portion size of 25% and cooked Lentil-based food at a portion size of 50%, of said breakfast; and a principle meal comprising 25% of each of raw salad, cooked vegetable, grain and lentil-based food.

Embodiments herein include five distinct progressive phases: Adjustment, Acceleration, Inner Transformation, Athletic Specialization, Individualization and Independence phase. The pace and duration of the phases is based on subject's requirement and health condition. The start of each phase is marked by attending the monthly group sessions.

Embodiments herein, preferably include assessing subject's condition in one or more group sessions. In an embodiment, the subject's health improvement is determined in one or more of such group sessions, preferably based on the markers at each stage. In an embodiment, the method includes 1 to 12, or more of such group sessions depending on the subject. The group session is preferably performed at the beginning of each phase. The group therapy includes a discussion of the subject with one or more health care professional/doctor for the subject, with or without other participants or subjects. Such sessions generally involve phase wise education and implementation. Participation and attendance in group sessions has been observed to improve subject's condition and may therefore be considered a requirement in the method described herein. The term “subject”, as used herein refers to a mammal, particularly humans. It refers to any individual having unhealthy lifestyle. It refers to any individuals intending to have healthy lifestyle. It refers to individuals having or suspected of having any disorder which is generally recognized as lifestyle associated disorders. It also refers to individuals having lifestyle related complications such as stress, anxiety, poor diet and other complications based on sedentary lifestyle. In some embodiment, it also includes individuals intending to have a disease free and healthy lifestyle. It includes any individual having a family history of diabetes. In another embodiment, subject is a patient having diabetes. The term “lifestyle related disorder” or “lifestyle associated disorder” or “lifestyle disorders”, according to various embodiments herein, includes any disorder or ailment which is generally linked with a subject's lifestyle. It is a non-communicable disease primarily based on and/or is manageable by management of the subject's lifestyle or daily habits, including, but is not limited to, diabetes, metabolic disorder, obesity, cardiovascular disorders, and diabetes related complications. It includes disorders that may or may not result from an unhealthy lifestyle but may be prevented, reversed or managed by maintaining a healthy lifestyle. It includes complications such as stress, anxiety, poor diet and other complications based on sedentary lifestyle.

The group sessions are preferably performed at the beginning of each phase (pre-recorded or live) although they may be conducted more frequently. Such sessions generally involve phase wise protocol demonstration, education, and instructions for implementation. Following the group sessions, the subjects are encouraged to seek one-on-one consultation with their assigned experts for customized instructions. Since participation and attendance in group sessions have been observed to improve subject's adherence and by association their chances of reversal, they may therefore be considered a requirement in the method described herein. The spectrum of the group sessions can be said to cover diet, exercise, inner transformation, and medical information. Some of the group sessions may be followed by a question-and-answer interaction with the facilitator for further clarity.

### Adjustment Phase

The embodiments herein include the adjustment phase. The adjustment phase includes providing the subject with an alkalizing diet, providing the subject with an exercise protocol comprising circulation and activation exercises, and determining improvement in subject's health condition. The individually customized protocol is based on a specifically designed battery of markers including, but not limited to, subject's requirement of medication, fasting blood glucose level (mg/dl), post prandial glucose level, HbA1C level (%), BMI (Body Mass Index) (kg/m2), and subject's waist circumference (cm). In an embodiment, the diet and exercise protocol are adjusted based on BMI (<25 or >25). In an embodiment, the adjustment phase diet is a plant based vegan diet including a green smoothie; lentil (also generally referred to as “Lentil”) based breakfast; Nuts and Seeds and Disorder Specific diet for thyroid, liver function, renal, and cardiac conditions. The alkalizing diet, according to embodiments herein, includes a diet with no milk/milk products or animal products. It includes a green smoothie; breakfast (25% sprouts, 25% salads and 50% cooked Lentil-based items); nuts (preferably soaked) and seeds; and a portion of principle meal. The principle meal includes a portion of 25% each of raw salad, a portion of cooked vegetable, a portion of grain, and lentil based food, wherein said meal comprises anti-diabetogenic food, wherein calorie intake of said diet is in the range of 1300 to 1400. Table 1 provides a sample diet chart based on BMI for phase 1.

TABLE 1
Adjustment phase diet chart based on BMI.

| S. No. | Diet | <25 BMI | >25 BMI |
|---|---|---|---|
| 1 | Green | 23-25 BMI | 4 glasses minimum |
| | Smoothie | (2 glasses only) | for all 4 weeks. |
| | | 21-23 BMI | If possible 5-6 glasses. |
| | | (only 1 glass) | |
| | | <21 BMI | |
| | | (Fortified Smoothie) | |
| 2 | Main | When target weight drop | Carb counting important |
| | direction | is achieved number of | 1 gm/kg body weight id |
| | | meals may be increased | ideal |
| | | | (can begin with 2 gm. |
| | | | Progress to 1.5 gm) |

For Hypothyroid, High Uric Acid, Kidney Stones, High Creatinine, High Potassium get special diet charts/restrictions

The smoothie is preferably consumed by the subject on an empty stomach, first thing in the morning. The green smoothie, according to embodiments herein, is a juice comprising at least one green leafy vegetable selected from a group consisting of spinach, kale, lettuce, aambat chukka (also known as green sorrel); at least one fruit selected from a group consisting of apple, pear, guava, berries and kiwi; at least two herbs selected from a group consisting of curry leaves (also known as Murraya koenigii), betel (also known as Piper beetle) leaf, tulsi (also known as Ocimum tenuiflorum) or basil leaves and mint leaves. Spinach may be avoided for hypothyroid subjects. The green smoothie may further include a pinch of black pepper powder, cinnamon powder, turmeric powder, rock salt and juice of half a lemon. The portion size of each of the ingredients may be tailored as per requirement. In an embodiment, the typical green smoothie comprises fresh juice obtained by blending spinach, half of apple, curry leaves, mint leaves, a pinch of black pepper powder, cinnamon powder, turmeric powder, rock salt and juice of half a lemon. For 500 ml of green smoothie, the mixture is blended with 250-300 ml water. For subjects having BMI between 23-25, two glasses or portions of smoothie is prescribed. For subjects having BMI between 21-23, only one glass or portion of smoothie is prescribed. For subjects having BMI above 25, a minimum 4 glasses or portions of smoothie is prescribed. The micronutrient rich green smoothie has been observed to reduce acid and inflammation, promote fat, improve energy levels, improve digestion, and improve hemoglobin (Hb).

The alkalizing diet, according to embodiments herein include anti-diabetogenic foods including vegetable such as cabbage, bitter melon, cucumber, garlic, onion, coccinia, okra, wood apple, and banana stem; nuts such as walnut and almonds; seeds such as flax seeds, sunflower seeds, watermelon seeds, pumpkin seeds, and sesame seeds; stevia; super greens such as spirulina, moringa, wheat grass, barley grass, curcumin, cinnamon, cayenne and holy basil.

The meal comprising a portion of sprouts, salad, and cooked Lentil or Lentil based food may be provided in equal proportion (25% each) prescribed as a breakfast and/or lunch meal. The salad and sprouts may include a combination of anti-diabetogenic food ingredients such as green gram, chickpeas, cabbage, bitter melon, cucumber, garlic, onion, coccinia, okra, carrot, wood apple, sweet potato, banana stem, strawberries, banana, mango, apple, orange and cherry. The breakfast may include lentil-based food dishes such as gram flour pancakes, mixed lentil flat bread, lentil-based dumplings, green gram based steamed cakes, pancakes etc. Alternatively, other forms of lentil or lentil-based food products may be prescribed. The portion size of each meal and its components may be tailored as per requirements. In an embodiment, the salad comprises of 20 to 30% sprouts, 20 to 30% salad, and 30 to 60% cooked Lentil or Lentil based food. In an embodiment, the breakfast meal comprises a combination of about 40 to 50% of raw foods comprising salads and sprouts; and 40 to 50% cooked food prepared using either pulse/lentils.

Nuts and seeds, according to embodiments herein, may be prescribed as an evening snack. The portion size may be tailored as per requirement. In an embodiment, about 8 to 10 nuts and 2 to 4 tablespoons of seeds are prescribed.

The principle meal, according to embodiments herein, may be prescribed as lunch and/or dinner meal. In terms of nutritional balance, the diet comprises of 55-60% of carbohydrates, 15-20% proteins and 20-25% fats. In an embodiment, the principle meal includes a portion of raw salad, a portion of cooked vegetable, a portion of grain, and lentil-based food in equal proportion (25% each). fox tail, little, proso, etc.), pulses, legumes, barley, bajra, brown rice, jowar, green gram, Garbanzo and kidney beans.

Grains are selected from a group consisting of minor millets (e.g.: fox tail, little, proso, etc), pulses, legumes, barley, bajra, brown rice, jowar, green gram, Garbanzo and kidney beans.

Diet, according to embodiment herein, includes a daily dose of micro and macro-nutrients. The portion of the diet that is used in the various embodiments of the method herein may vary depending on the body types categorized as ectomorph and thin mesomorph (<25 BMI) and Obese Mesomorph and Endomorph>(25 BMI).

The exercise protocol in adjustment phase includes circulation and activation exercises. The circulation and activation exercise, according to embodiments herein, may include any exercise known to activate muscle fiber; reduce acid and inflammation and detoxify the human body. The circulation and activation exercise includes exercises selected from a group consisting of warm up, super brain yoga, soorya namaskaras, palm plank, push-ups and anti-gravity exercise (AGE: ascending and descending a stairway and nitric oxide dump) which may be tailored based on BMI (<25 OR>25). Nitric oxide dump includes alternate arm raise for about 20 times; squats, about 8 times; shoulder presses, about 20 times; and non-jumping jacks, about 20 times. Soorya namaskaras, or sun salutation exercise, is a generally known ancient Yogic Practice consisting of about 12 body poses performed in succession.

In an embodiment, the warm-up includes a combination of dry rubbing, joint movement, energy shake, and acupressure tapping. Dry rubbing may be performed by rubbing the subject's body with a dry cloth. Joint movement, energy shake and acupressure tapping may be performed as per generally known methods. The duration and time of exercise varies depending on the subject Table 2 provides a sample exercise chart for phase 1 based on BMI.

TABLE 2
Adjustment phase exercise chart based
on BMI (Morning Exercise Variation)

| S. No | Exercise | <25 BMI | >25 BMI |
|---|---|---|---|
| 1 | World's Best Warm Up | Yes | |
| 2 | Super Brain Yoga (Squats) | Upto 20 in 1 set by end of 4 weeks | |
| 3 | Soorya | Start with 3. | |
| | Namaskaras(Chair/Regular) | Within 2 weeks 12. | |
| | | Within 4 weeks 24. | |
| 4 | Walk (20 minutes only) | Start with 1-minute | Walk or cycle |
| | | jog:2-minute walk. | 20 minutes |
| | | End with 1-minute | only |
| | | jog:1-minute walk. | |

| | | |
|---|---|---|
| 5 | Palm Planks (Pose 5 in | 30 secs × 3 reps |
| | Soorya Namaskar) | Increase to 45 secs × |
| | | 3 reps within 2 weeks |
| | | 60 secs × 3 reps within 4 |
| | | weeks |
| 6 | Pushups | ×3 sets (daily) |
| | (Normal/Modified/Wall) | |

In an embodiment, the anti-gravity exercises (AGE) i.e. post meal exercises are performed 1 hour 45 min from the start of every meal e.g.: post breakfast, lunch, and dinner. AGE includes a combination of staircase climbing and Nitric oxide dump performed for a total for 10-15 minutes. Table 3 provides a sample protocol for exercise chart based on BMI for post meal exercises.

TABLE 3
Adjustment phase exercise chart based
on BMI (Post Meal Exercise Variation)

| S. No. | Exercise | <25 BMI | >25 BMI |
|---|---|---|---|
| 1 | Stair | With (5-10 kg) or | Non-Weighted |
| | Climbing | without weight × 150 | Start with |
| | AND/OR | steps × 3 times (meal) | 100 steps × 3 |
| | Nitric Oxide | | times going |
| | Dump (NOD) | | upto 300 steps × |
| | | | 3 times (meal) |
| 2 | | 3 sets × 10 reps × | 3 sets × 20 reps × |
| | | 3 times (meal) | 3 times (meal) |
| | | (with 1-liter bottle | (with 1-liter bottle |
| | | in each hand) | in each hand) |
| 3 | Heart | NOD or max 100 steps without weights | |
| | Subjects <30% | | |
| | EF | | |

>30 BMI=100 steps once a day on alternate days. The embodiments herein include determining improvement in subject's health condition. Markers which indicate the progress or improvement in adjustment phase includes, reduction in acidity, reduction in body weight, improvement in subject's energy level, improvement in stamina, improvement in body flexibility, improvement in strength, decrease in blood sugar level and decrease in requirement of medication. In an embodiment, markers include subject's medication requirement, weight, fitness levels, stress levels, fasting blood glucose level (mg/dl), post prandial glucose level, HbA1C level (%), BMI (Body Mass Index) (kg/m2), and subject's waist circumference (cm).

In an embodiment, the method comprises monitoring all the following parameters: BMI, energy level, stability in body weight, reduction in body weight, improvement in strength, decrease in blood sugar level and decrease in requirement of medication. According to embodiments herein, a 5 point drop in BMI or achieving a BMI of 23 and below is considered an improvement. For sugar levels, consistent stability of fasting sugar readings below 110 and PP<140 would be considered for reduction in subject's medication. Relative ease in performing the phase 1 exercises is considered as an improvement in strength, according to embodiments herein.

The adjustment phase helps in removing IGF molecules; alkalize the body by removing acidic food; reduce fat by reducing calories; adds antioxidants, minerals, and vitamins concentration in body; improve circulation of lymph and blood from skin, joints, muscle and deep tissues and finally activate full body muscles, joints and deeper tissues.

In an embodiment, the duration of adjustment phase is between 4 to 8 weeks depending on the date of joining of the program.

### Acceleration Phase

The embodiments herein elucidate an acceleration phase. The acceleration phase includes providing or prescribing the subject a cleansing diet and an exercise protocol comprising yoga and strengthening exercises, while monitoring improvement in subject's health condition. The individually customized protocol is based on a specifically designed list of markers which may be determined using standard tests. The tests include generally known test for monitoring of the subject's medication, fasting blood glucose level (mg/dl), post prandial glucose level, HbA1C level (%), BMI (Body Mass Index) (kg/m2), and subject's waist circumference (cm). In an embodiment, the diet and exercise protocol are adjusted based on BMI (<23 or >23). In an embodiment, the diet is a plant based vegan diet comprising green smoothie (up to a volume of about 1000 mL); lentil (also generally referred to as “Lentil”) based breakfast; Nuts and Seeds, juice feasting and principal meal. The main meal, according to embodiments herein, may be prescribed as lunch and/or dinner meal as described in the adjustment phase. The diet protocol includes intermittent fasting, juice feasting, and water fasting. Table 4 provides a sample diet chart based on BMI.

TABLE 4
Acceleration phase diet chart based on BMI.

| S. No. | Diet | <23 BMI | >23 BMI |
|---|---|---|---|
| 1 | Green | Fortified or Anabolic Smoothie | Regular Smoothie |
| | Smoothie | (1 or 2 glass only) | 23-25 BMI: 2 glasses only |
| | | (Add 4 almonds/nuts, 2 tsp oil, | >25 BMI: 4 glasses or more |
| | | 2 tsp seeds and 2 tsp protein | |
| | | powder) | |
| 2 | Juice Feasting | Once a week | Once a week 10-14 glasses |
| | (JF)/Water | 21-23 BMI: Full day JF | or Water Fast (WF) |
| | Fasting (WF) | <21 BMI: Half day JF | |
| | | (have dinner) | |
| | | Type 1s: can have juices | |
| | | in between meals | |
| 3 | Intermittent | NO IF Protocol | Complete IF Protocol |
| | Fasting (IF) | (As don't need to burn fat) | 1st week: 3 2 3 2 3 2 JF/WF |
| | | 3, 4, 5 or 6 meals a day as | 2nd week: 2 2 2 2 2 2 JF/WF |
| | | given by Diet Experts depending | 3rd week: 2 1 2 1 2 1 JF/WF |
| | | on BMI and weight targets | 4th week: Long fast for 48 hours+ |

Hypothyroid, High Uric Acid, Kidney Stones, High Creatinine, High Potassium get your special diet charts/restrictions from your respective Diet Experts

The cleansing diet is a continuation of the phase 1 diet with the same dietary principles. Additionally, intermittent or water fasting or/and juice feasting for incremental durations of half a day, one day, or longer fasting is specifically provided or prescribed as per needs of the subject.

Juice feasting includes consuming juices such as red juice, green juice, and white juice. In an embodiment, the red juice comprises suitable quantities of the following: tomato, carrot, red capsicum and yellow capsicum. The green juice is of two types, one or both of which may be prescribed to the subject. The green juice comprises suitable quantities of ash gourd, apple, capsicum, ridge gourd and lemon juice. Alternatively, the green juice comprises suitable quantities of bottle gourd, green sorrel/Chenopodium, coriander leaves and lemon juice. The white juice comprises suitable quantities of bottle gourd, cucumber, and lemon juice. Table 5 depicts the different types of juices and their composition.

TABLE 5
Juices and their composition.

| | Juice | | Time of |
|---|---|---|---|
| S. No. | type | Composition | consumption |
| 1 | Red | 800 gm tomato, 250 gm carrot, 1 red capsicum, 1 | Morning |
| | juice | yellow capsicum (500 ml) | |
| 2 | Green | 500 gm Ash gourd, half apple, 4 big capsicums, | Afternoon |
| | juice 1 | 1 ridge gourd, half lemon juice (500 ml) | |
| 3 | Green | 700 gm bottle gourd, 2 cups green | Afternoon |
| | juice 2 | sorrel/Chenopodium, 1 cup coriander, half | |
| | | lemon juice (500 ml) | |
| 4 | White | 500 gm bottle gourd, 500 gm cucumber, half | Evening |
| | juice | lemon juice (500 ml) | |

The smoothie is provided or prescribed as previously described herein. The cleansing diet, according to embodiments herein, includes iron and vitamin rich meals. Iron rich meals include Spinach, broccoli, almonds, sunflower seeds, fenugreek, carrots, tomatoes, lentils, green peas, chickpeas, soya beans, muesli, etc. Other iron rich foods may also be prescribed. Substances such as Coffee, tea, calcium supplements and milk are preferably avoided as they lead to low iron absorption.

The meal comprising a portion of sprouts and salad is provided or prescribed as a breakfast and/or lunch meal as described previously herein. Nuts and seeds, according to embodiments herein, may be provided or prescribed as an evening snack as described in the adjustment phase. The portion size may be adjusted as per subject requirement. The principle meal, according to embodiments herein, may be provided or prescribed as lunch and/or dinner meal as described in the adjustment phase.

As a hunger suppressant “Royal Water (Shahi Pani)” is advised which is a drink comprising water with 1 tsp sabja/chia seeds, rock salt, and a few strands of kesar and juice of a medium sized lemon. 1 cup at a time is preferable. For High Potassium patients, Sabja/Chia, Lemon and rock salt is not recommended.

In an embodiment, the method includes intermittent fasting (IF) protocol in the acceleration phase that is based on BMI. Shahi pani acts as a hunger suppressant during intermittent fasting in the acceleration phase. Intermittent fasting is an eating pattern where the subject switches between periods of eating and fasting. Fasting may include water fasting (WF) and/or juice feasting (JF). Intermittent fasting pattern depends on BMI of the subject. Table 6 provides a sample intermittent fasting protocol.

In an embodiment, IF protocol for BMI>23 includes a first week of 3-2-3-2-3-2 of JF or WF, a first week of 2-2-2-2-2-2 of JF or WF; a third week of 2-1-2-1-2-1 of JF or WF; and a 4th week of long fast, wherein 1,2 or 3 indicates the number of cooked meals in a day of the week). Table 6 indicates the protocol for intermittent fasting for various BMI.

Breakfast comprises of lentil-based items along with sprouts and salad (50% cooked and 50% raw); whereas lunch and dinner include 25% grain, 25% daal, 25% subji and 25% salad. Brunch is same as lunch with 25% grain, 25% daal, 25% subji and 25% salad (timing for brunch can be between 9 am to 1 pm); sprouts can be included with brunch keeping daal and sprouts within 25% of the total meal. To minimize insulin secretion, ideally smoothie on Tuesday, Thursday and Saturday should be without the fruit (cucumber, tomato or bottle gourd instead of fruit) and can be consumed in the morning or afternoon. In between meals may be prescribed, comprising of water with sabja or chia seeds, beverages such as Cinnamon Tea/Herbal Tea, Green/Black Tea, Black Coffee/Coffee with coconut milk or coconut oil and soaked almonds, walnuts and seeds. During the second week of IF, 2 2 2 2 2 2 JF/WF pattern in followed (2 cooked meals i.e. brunch and dinner in a day). These meals follow the same FFD principle as in the first week. Smoothie on all days should be consumed without fruits and can be consumed in the morning or afternoon. In between meals same as first week may be provided or prescribed. Further in an embodiment, third week includes IF 2 1 2 1 2 1 JF/WF pattern. One meal per day is according to the subject's comfort level i.e. either brunch or dinner. These meals follow the same FFD principle as in the first week. Smoothie on all days should be consumed without fruits and can be consumed in the morning or afternoon. In between meals same as first week may be prescribed. The fourth week includes a long fast (minimum 48 hours) with single meals on other days. Benefits of IF include:—significant reduction of insulin level in blood thereby facilitating in burning of fats; weight and belly fat reduction; and multifold activation of body's internal healing mechanism. Fasting protocol is not preferred for Type 1 diabetic subjects, children below 18 years, pregnant and breast-feeding women and subjects having gastrointestinal reflux disease.

TABLE 6
Intermittent Fasting (IF) protocol

| S. No. | <23 BMI | >23 BMI |
|---|---|---|
| 1 | 21-23 BMI: 3 3 3 3 3 3 3 JF | Complete IF Protocol |
| | <21 BMI: 3 3 3 3 3 3 3 JF | 1st week: 3 2 3 2 3 2 JF/WF |
| | (can have dinner) | 2nd week: 2 2 2 2 2 2 JF/WF |
| | (OR as per 4 to 6 meals a day) | 3rd week: 2 1 2 1 2 1 JF/WF |
| | Depending on BMI and weight | 4th week: Long Fast |
| | targets | |

| | |
|---|---|
| 2 | >25 BMI who still need weight loss |
| | First week options: 2 2 2 2 2 2 JF or 2 1 2 1 2 1 JF |
| | Second week options: 2 1 2 1 2 1 JF 2 1 or 1 1 1 1 1 1 JF |
| | Third week: 1 1 1 1 1 1 JF |
| | Fourth week: 1 long fast (minimum 48 hours) |
| | with single meals on other days |
| 3 | STUBBORN WEIGHT |
| | First week: 1 1 1 1 1 1 JF |
| | Second week: 1 long fast (minimum 48 hours) |
| | with single meals on other days |
| | Keep alternating the above combination in |
| | subsequent weeks or go for 1 1 1 1 1 1JF |

The exercise protocol in acceleration phase includes Yoga and strengthening exercises. Yoga and strengthening exercises include exercises selected from a group consisting of cleansing processes, posture correction, special yoga sequence, strength building and resistance band exercises which are tailored based on BMI (<25 OR>25).

In an embodiment, the exercise protocol includes warm up, palm plank, pushups, walking or jogging, cleansing processes, posture correction, special yoga sequence, strength building and resistance band exercises and anti-gravity exercise such as staircase climbing and nitric oxide dump. In an embodiment, the cleansing processes, also known as Dhautikriyas (specific cleansing processes, generally known in the yoga, for the oral cavity and head region), such as cleansing of roots of teeth, mouth palate, roots of tongue, ears, eyes, nose, anus, etc. The cleansing processes are to be performed by the subject, preferably in inverted foot posture. Special yoga sequence includes a combination of yoga asanas or yoga exercises such as breathing exercise, Kapalbhati, Agni-Saar Kriya, and Yogasanas-ArdhMatsyendrasana, Paschimottanasana, AdhomukhiSwanasana, Paadahastana, Baalasana, etc. The yoga asanas may be performed as per generally known practices. Alternatively, any basic yoga exercises may be performed.

In an embodiment, the strength building exercises include lightweight training and/or resistance band (upper body focused) exercises performed for 10 minutes about 15 repetitions each, 3 times a week.

In an embodiment, the phase 1 exercises and AGE are continued albeit for a reduced duration.

The duration and time of exercise may vary depending on the subject. In an embodiment, the morning exercise is performed for a duration of 10 to 60 mins. Table 7 provides exercise chart based on BMI for morning exercise variation.

TABLE 7
Exercise chart based on BMI (Morning exercise variation)

| S. No. | Exercise | <25 BMI | >25 BMI |
|---|---|---|---|
| 1 | World's Best Warm Up | Yes (<5 minutes) | |
| | (Short Version) | | |
| 2 | Super Brain Yoga | 10 × 3 times per day (daily) | |
| | (Squats) | | |
| 3 | Soorya | 6-12 reps (daily) | |
| | Namaskaras (Chair/ | Focus on breathing and form | |
| 4 | Resistance Band | 15 minutes (daily) | |
| | | (1-4 exercise. 15 reps each hand × 3 sets) | |
| 5 | Walk/Jog | Nil | 30 minutes on non JF |
| | | | & Water Fast days |

| | | |
|---|---|---|
| 6 | Pushups | 10 × 3 sets (daily) |
| | (Wall/Normal/Modified) | |
| 7 | Palm Planks | 1-minute × 3 sets (daily) |
| | (Pose 5 in Soorya | |
| | Namaskar) | |
| 8 | Whole System | 8 breaths × 8 times a day |
| | Breathing | |
| 9 | Kapalbhati | Start-25 counts from each nostril. 50 from both. |
| | | Increase to 50 from each and 100 from both in 1 |
| | | week |
| | | Upto 75 from each and 150 from both within 2 |
| | | weeks |
| 10 | Agni-saar Kriya | 3 times (for max duration each time) |
| 11 | Asana Practice | 30 seconds × 3 sets each |
| | (Diabetes and Other) | (ArdhMatsyendrasana, Paschimottanasana, Adhomukhi |
| | | Swanasana, Paadahastana, Baalasana) |

In the embodiment, the method comprises providing an exercise protocol comprising post meal exercise of Anti-Gravity Exercise (AGE). In an embodiment, the post meal exercise is performed for 8-10 mn, 1.5 hours to 2 hours after start of every meal e.g.: post breakfast, lunch, and dinner. Table 8 provides exercise chart based on BMI for post meal variation.

TABLE 8
Exercise chart based on BMI (Post meal exercise variation).

| S. No. | Exercise | <25 BMI | >25 BMI |
|---|---|---|---|
| 1 | Stair Climbing | With or without weight | Preferably weighted thrice a day |
| | And Nitric | once/thrice a day | 5 kgs × 100 steps × 3 times |
| | Oxide Dump | depending on doctor's | >30 BMI |
| | (NOD) | opinion | (Non weighted based |
| | | 5-10 kgs × 100 steps | on one's comfort) |

| | | |
|---|---|---|
| | | 3 sets × 10 reps post each meal |
| | | (with 1 liter bottle in each hand) |
| 2 | Heart | NOD or max 100 steps without weights |
| | Subjects <30% | Other special |
| | EF | |

The embodiments herein include monitoring subject's health status. Markers which indicate the progress or improvement in acceleration phase include faster reduction in body weight; improved energy levels; improvement in posture and breathing; further improvement in flexibility and body awareness; alleviation of any acidity, pains, digestions problems, etc.; and decrease in blood sugar level and decrease in requirement of medication. In an embodiment, the subject may be subjected to an extended period of acceleration phase if the desired reduction in BMI (either a 5-point drop in BMI or target BMI below 23) is not achieved. The total duration of this phase may vary from 30 to 90 days. The transition to the next phase is marked by the monthly group session.

The acceleration phase helps in detoxification; enhancing body's repair system, rehydration of body; inducing natural ketosis, deeper cleansing and initiating strength building.

### Inner Transformation Phase

The embodiments herein include the inner transformation phase. The inner transformation phase does not include a separate diet and exercise protocol. Depending on the BMI of the subject, the phase includes prescribing the subject with a cleansing diet or a muscle building diet, providing the subject with an exercise protocol comprising of yoga and strengthening exercises or sampling and athletic specialization exercises, whilst providing the subject with a protocol for meditation and release of stress, and determining improvement in subject's condition. In an embodiment, the diet and exercise protocol are adjusted based on BMI. If a subject has >23 BMI or has not yet achieved a 5-pointdrop in BMI, alkalizing diet and acceleration phase exercise must be continued while for <23 BMI, muscle building diet and exercise is prescribed.

In an embodiment, if the subject has not achieved the desired BMI level (as described above), cleansing diet is continued as has been described in detail in the acceleration phase. It includes a green smoothie; breakfast (25% sprouts, 25% salads and 50% cooked Lentil-based items); nuts (preferably soaked) and seeds; juice feasting/water fasting/intermittent fasting as per BMI protocol. According to the embodiment herein, the principle meal may be in the form of lunch and/or dinner with description given previously.

In an embodiment herein, if the diet is followed as per athletic phase, then the diet (muscle building) is planned as per body type i.e., Ecto, Meso and Endo. In an embodiment herein, the muscle building diet protocol includes increasing number of meals and calories. Further, the embodiment also includes protocol for those having sensitive sugar levels by following an incremental approach wherein the number of meals is increased on a case to case basis.

In an embodiment herein, for the athletic specialization phase, the calorie intake and Carbohydrate:Protein:Fat ratio are followed as per body type. For all the three body types 250 ml of Anabolic smoothie (preparation described in adjustment phase) is advised. The breakfast (cooked lentil-based meals), lunch and dinner (25% of lentil, grains, cooked vegetables and salads each) are portioned according to serving size for each body type restricted oil intake. comprises of fruits, while for early evening fruits, nuts and seeds are recommended. Grain and lentil or lentil-based meal are allowed during late evenings. For muscle building, 20-30 gm of supplementary protein powder is recommended for all three body types. The protocol also includes consumption of plant-based milk at bedtime. Table 9 provides a sample diet chart for muscle building based on body type.

TABLE 9
Diet chart for muscle building based on body type.

| S. No. | Components | ECTO | | MESO | | ENDO | |
|---|---|---|---|---|---|---|---|
| 1 | Calories (in Kcal) | 2200 | | 2000 | | 1800 | |
| 2 | Carb:Protein:Fat (in grams) | 300:80:75 | | 270:75:70 | | 240:75:60 | |
| 3 | Anabolic Smoothie | Y (250 ml) | | Y (250 ml) | | Y (250 ml) | |
| 4 | Breakfast Cooked Lentil based | 2 | servings | 2 | servings | 1 | serving |
| 5 | Mid-morning | Fruit | | Fruit | | — | |
| 6 | Lunch (25% principle) | 2 | servings | 1.5 | servings | 1.5 | servings |
| 7 | Early evening Fruit, Nuts, Seeds | Yes | | Yes | | Yes | |
| 8 | Late evening | Grain + Lentil | | Grain + Lentil/Lentil | | Lentil Based snack | |
| 9 | Dinner (25% principle) | 2 | servings | 1.5 | servings | 1.5 | servings |
| 10 | Oil | 8 | tsp | 7 | tsp | 6 | tsp |
| 11 | Protein Powder | 20-30 | gms | 20-30 | gms | 20-30 | gms |

| | | |
|---|---|---|
| 12 | Bedtime | Plant based Milk |

For those desiring additional weight loss or for those whose weight loss is stagnant, the use of SNS (Smoothie, nuts, and soups) may be suggested. This is a meal replacement plan and involves consuming 2 glasses of smoothie in the morning using a low GI fruit like Apple, Berries, Kiwi, Dragon Fruit, Pear, Apple, or Guava; 25-40 nuts consumed in lunch. These must include at least 10 soaked walnuts and almonds (peeled); the rest can be Cashews, Pistachios, Macadamia Nuts, Brazil Nuts, Pecans, etc. The subject can also take 1 or 2 Dates, OR 5 to 10 Raisins, along with the nuts (however, if sugars are high, these should be avoided). For dinner, 400-500 ml thick soups are recommended (Avoid adding more than 50 gm of high GI vegetables like Beetroot, Carrot, or Pumpkin, in Soup. The idea is to use about 250 gm of vegetables for making 500 ml soup.

Further, the embodiments herein include monitoring and enabling weight gain for subjects with <21 BMI. It further includes setting a target to increase weight by 1-1.5 kg per month while measuring weight every day after bath; analyzing the diet intake that has contributed to achieving weight gain and calculating the extra that would be needed. If the weight is still stagnant or reduces, calorie intake can be increased to 300 by consuming additional 2 tsp oil, 2 fruits and grain. In addition, the number of meals may be increased to 4, 5, or 6 with intake of anabolic smoothie, 8 tsp oil and seeds each, 20 nuts and 3-4 fruits. The main goal of the embodiment herein is to increase the feeding window and reduce fasting window. Strength building exercises such as Dumbbells, Bands, Push Ups, Squats, Sit Ups and Weighted AGE (WAGE) should be performed. A ratio of 3:2:1 of Strength:Yoga:Cardio or a combination thereof should be followed with reduction or stopping of cardio exercises (walking, running, AGE).

There are no specific inner transformation phase exercises as mentioned before; acceleration or athletic sampling and specialization phase exercise can be followed during this phase. The acceleration phase exercises have previously been described in the acceleration phase. It includes yoga and strengthening exercises according to the BMI of the individual (<25 OR>25). In an embodiment herein, the acceleration phase exercises are divided into morning and post meal exercises.

In an embodiment herein, the athletic specialization phase exercises i.e., sampling and specialization exercise, are aimed to reveal the subject's own athletic identity. These may be Yogi/Yogini, Runner, Cyclist, Gymmer/Strength, Dancer and Swimmer. The subject may be asked about their athletic history, whether they enjoy team sports or individual sports, gym, or yoga. The subjects can select the athletic identity of their choice along with the duration per week.

In an embodiment, the sampling includes key considerations such as age, BMI, goals, logistics and skills. Table 10 provides a sample plan based on body type.

TABLE 10
Sampling exercise based on body type.

| | Body Type | | | |
|---|---|---|---|---|
| | Endomorphs/Mesomorphs | | Lean Mesomorphs/Ectomorphs | |
| | BMI | | | |
| | | Recommended | | Recommended |
| | BMI >25 | List | BMI <25 | List |
| Starting | Avoid running or | Cycling | Alternate day running | Running |
| Exercise | skipping that put | Yoga | or skipping is | Yoga |
| Type | pressure on knees | Gym | acceptable | Gym |
| | and back | Zumba/ | Avoid Heavy | Cycling |
| | Avoid Impact Sports | Dance Or | weightlifting | Zumba/ |
| | like squash, basketball | any sport | without proper | Dance Or |
| | | | guidance | any sport |

Further, the embodiment includes enquiring the subject's top 3 exercises that he/she can sample for next three months. The first choice comprises of 3 hours/units per week (Monday, Wednesday and Saturday), second choice of 2 hours/units per week (Tuesday, Thursday) and lastly third choice of 1 hour/units per week (Friday). Sunday is kept for rest, hydration and stretches.

The inner transformation phase focuses on improvement of emotional quotient (EQ) for the release of stress, improving awareness and inner healing. EQ is the ability to perceive and evaluate emotions in positive ways for stress release, effective communication, overcoming challenges, have empathy for others and managing conflicts. The phase comprises of water charging, gratitude journaling, book of life, anger/grief and forgiveness meditations, right brain activities, selflove and appreciation, completion letters to loved ones, cultivating mindfulness, mindful and grateful of challenges and Brahma Viharas. The Brahma Viharas are 4 states of mind (loving-kindness, compassion, empathetic joy and equanimity) and meditation practices according to Buddhism.

The steps for performing meditation include sitting in whole brain/whole heart posture, i.e. sitting with arms and legs crossed, with closed eyes; taking deep breaths, slowly focusing down from the top of the head and traveling behind the eyes, through the heart and abdomen to the base of the spine and getting in touch with the vibrations of the body; remembering in detail, the past stressful experience or the future anxiety by seeing the scenes, hearing the words, feeling the emotions and in the end exaggerating them; creating a strong intention to release this stored energy and breathe out in breaks repeatedly till the pictures fade away and/or feelings dissolve; and lastly focusing on heart to receive deeper guidance on why this happened, what was the purpose (if any) and what's the direction/action to be taken now.

Further the embodiment includes short meditation and affirmation steps called I can! I will!! Meditation. The first step is taking 10-30 long breaths by inhaling through nose and exhaling through mouth. Step 2 is taking one long breath in and holding for as long as comfortable (for high BP individuals less than 15 seconds while others can go up to 60 seconds). Step 3 of the process is to completely breathe out and avoid breath in again for next 15-30 seconds. Step 4 comprises of focusing attention to the center of the brain, going deep into the throat and lastly lingering in the area of heart. The last step includes saying and feeling I can! I will!! till one feels positive and determined. If an individual has difficulty in following these steps in detail, shorter versions may be repeated many times a day.

In an embodiment herein, the inner transformation protocol includes sleeping early and getting up early (ideal time may be 5 am) with 4 steps of 20 mins each. Step 1 includes physical and energy cleansing, comprising of, emptying bowels ending with Ganesh Dhauti (process of cleansing in and around the anus after emptying bowels so that any remaining faecal matter is also disposed off), brushing teeth ending with Dhauti Kriyas, water charging, and drinking it peacefully. Step 2 includes emotional and mental cleansing by Journal Writing (day to day thoughts, feelings, events, actions and even dreams). Step 3 is the physical and energy activation consisting of world's best warm up and Nitric Oxide Dump (1-2 cycles), thereby, immediately reducing cortisol (stress hormone) and increasing happy hormones such as serotonin, and dopamine. The last is step 4 i.e., emotional and mental activation, with 3 months of a variety of inner transformation practices to be followed every day.

Further the embodiment includes becoming aware (of one's beliefs/patterns), taking the life experiences lightly and not seriously, checking personal willingness for change (moving to 100% willingness), clarifying the end outcome (SAAF format). SAAF stands for Scene, Actors, Actions and Feelings. The SAAF goals include, clearing GTT (Glucose Tolerance Test), losing 10 kg weight and being free from tablets and insulin. These goals need to be visualized, heard and felt to instill message deep in the subconscious and help in achieving one's goal.

The embodiments herein, include monitoring subject's status. Markers which indicate the progress or improvement in inner transformation phase, include feeling lighter due to release of past emotions; increased ability to handle existing challenges in personal as well as professional life; experiencing deep inner positivity with feeling loved, confident, optimistic, elated and at peace; feeling of greater connectivity or bonding with self as well as with others; absolute clarity of weight and fitness goals (strength/stamina/flexibility) with actions needed for accomplishing them; being highly motivated in achieving one's goals; progressing to the next phase by reducing or gaining weight as per diet plan and last and foremost reduction in sugar levels and decrease in requirement of medications especially if it is linked to stress/anxiety. The inner transformation phase aids in improving EQ by release of stress, improves awareness and inner healing.

One of the aims by the end of the inner transformation phase is to maintain sugar levels below 140 for at least 18 hours per day which is called good control; below 140 for 21 hours being very good control. HbA1c is said to be in safe range if one achieves 18/24-hour sugar window for 95% days.

In an embodiment, the duration of inner transformation phase is about 12-13 weeks.

### Athletic Specialization Phase

The embodiments herein include the athletic specialization phase. The athletic specialization phase includes providing the subject with a muscle building diet, providing the subject with an exercise protocol comprising sampling and specialization exercises; and determining improvement in subject's health condition. The subject should have BMI of 23 or a drop of 5 BMI from the initial count for entry into this phase. In an embodiment, the muscle building diet is planned according to body type i.e., Ecto, Meso and Endo and basis level of tolerance to glycemic load by increasing number of meals and calories (1500-1800 calories). The diet protocol along with the table (Table 9) has been described in detail in the inner transformation phase.

Further the embodiment includes Carbohydrates:Protein:Fat ratio distribution in meals according to the BMI category (Table 11).

TABLE 11
Carbohydrate:Protein:Fat ratio based on
BMI category for muscle building diet.

| | BMI | Carb:Protein:Fat | Multiplication | |
|---|---|---|---|---|
| S. No. | Category | Ratio | Factor w.r.t Weight | Calories |
| 1 | <21 | 4:1.25:2 | Weight × 3 | |
| 2 | 21-23 | 4:1.25:1 | Weight × 3-3.5 | 1800+ |
| 3 | 23-25 | 4:1.25:1 | | 1800+ |
| 4 | >25 | 4:1.25:1 | Weight × 2.5 | 2000++ |

Table 12 highlights the main difference between phase 1 and phase 4 diet. As can be seen from the table, more carbohydrates and proteins are included in the muscle building diet along with high calorie intake as compared to the alkalizing diet.

TABLE 12
Main Differences between Phase 1 and 4 Diet

| | | Calorie ratio with | |
|---|---|---|---|
| Phases | Carb:Protein:Fat Ratio | weight | Calories |
| Phase 1 | 4:1:1 | Weight X 2-2.5/1.5 | 1300-1400 |
| Phase 4 | 4-5:1.25:1; 4 to 5:1 to | Weight X 3-3.5/2.5 | 1800-2200 |
| | 1.25:1 to 1.25 | | |

The athletic specialization phase exercises focus on strength, stamina, and flexibility. In an embodiment, the sampling & specialization exercises are based on the athletic identity formula of 3:2:1. Brewer et al., defined athletic identity as the degree to which an individual identifies with the athlete role (1993). The participants can select the athletic identity of their choice along with the duration per week. The identities are: Yogi/Yogini, Runner, Cyclist, Gymmer/Strength, Dancer and Swimmer. The first choice comprises of 3 hours/units per week (Monday, Wednesday and Saturday), second choice of 2 hours/units per week (Tuesday, Thursday) and lastly third choice of 1 hour/units per week (Friday). Sunday is kept for rest, hydration and stretches.

In an embodiment, the athletic specialization phase also includes imparting knowledge to the subjects about medications and their mode of action.

The athletic specialization phase helps in improving energy storage via ideal calorie partitioning, gradually increasing muscle surface area and reducing fat (muscle to fat ratio); this leads to reshaping of the body by repetition of specific exercises, identifying DNA (Deep Natural Alignment) to particular exercise forms through sampling technique and finally improvement in balancing strength, stamina and flexibility by including variety of exercises.

In an embodiment, the markers for the progress of athletic specialization phase includes deeper enjoyment of exercising; better muscle to fat ratio; alteration in body shape in accordance to the specialized exercise form; lesser dependence on AGE for lowering PP sugars; increase in confidence, clarity, energy and vibrancy and finally ability to explore food variations without drastic change in sugar levels.

In an embodiment, the duration of athletic specialization phase is about 4 to 5 weeks although the journey from sampling to specialization may take longer depending on an individual.

### Individualization & Independence

The embodiments herein include individualization and independence phase.

The individualization and independence phase includes providing the subject with a muscle building diet chart to sustain reversal and phase 4 athletic identity. The muscle building diet has been described in detail in the athletic specialization phase.

In an embodiment, the exercises for individualization and independence phase are aimed at achieving fitness goals.

In an embodiment, the duration of individualization and independence phase is about 1 to 6 months.

In an embodiment herein, the duration of each phase may vary depending upon the subject's health, progress, requirements, and flexibility.

In an embodiment, the outcomes are measured by observing time taken to discontinue diabetes medication (OHAs/insulin), fasting and post prandial blood glucose level (mg/dl), HbA1C level (%), subject's BMI, and subject waist circumference (cm).

By the end of the program the subjects can experience optimal health in the following aspects: reduction in acidity, pain, digestion problems (if any); reduction in weight (for those who need to reduce); raised energy levels; improvement in stamina; improvement in flexibility; improvement in strength; feeling lighter, hopeful, positive and reduced/controlled sugar levels and/or drop/stoppage of medication and attaining an ideal BMI and body composition.

### Experimental Study Data

A controlled intervention study was conducted in Pune city, India with patients enrolling from all over the world through online registrations. A total of 3926 participants completed a one-year program. The inclusion criterion was Type 2 diabetic/prediabetic status. The duration of the intervention was 12 months. The intervention for the last batch starting in July 2020 was completed in June 2021. All the participants underwent the 5 Phases of the intervention program, namely-Adjustment, Acceleration, Inner Transformation, Athletic Specialization, and Individualization & Independence phase. The intervention was in phased manner conducted through 12 groups sessions.

### Observations

The one-year controlled intervention study was undertaken by 7 different groups. The observations and subsequent results from the experimental data study for each of the groups are described by the tables and corresponding figures provided herein.

### Group 1

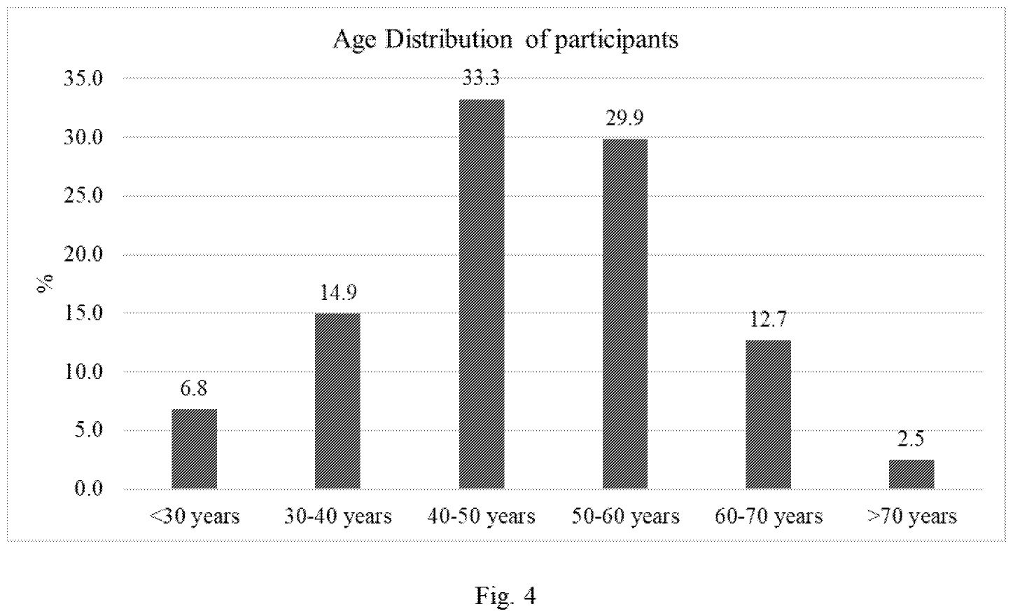

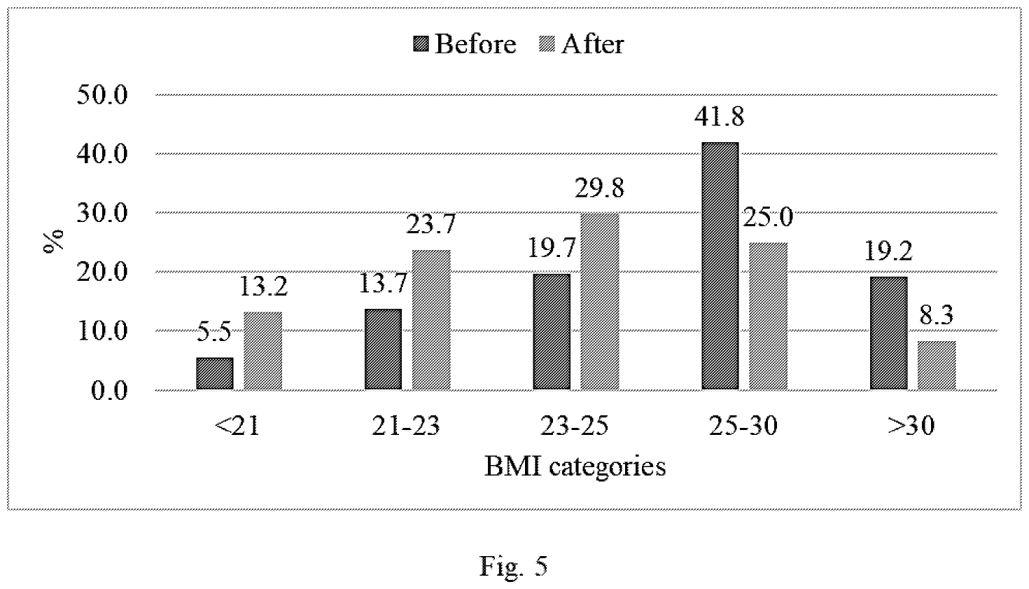

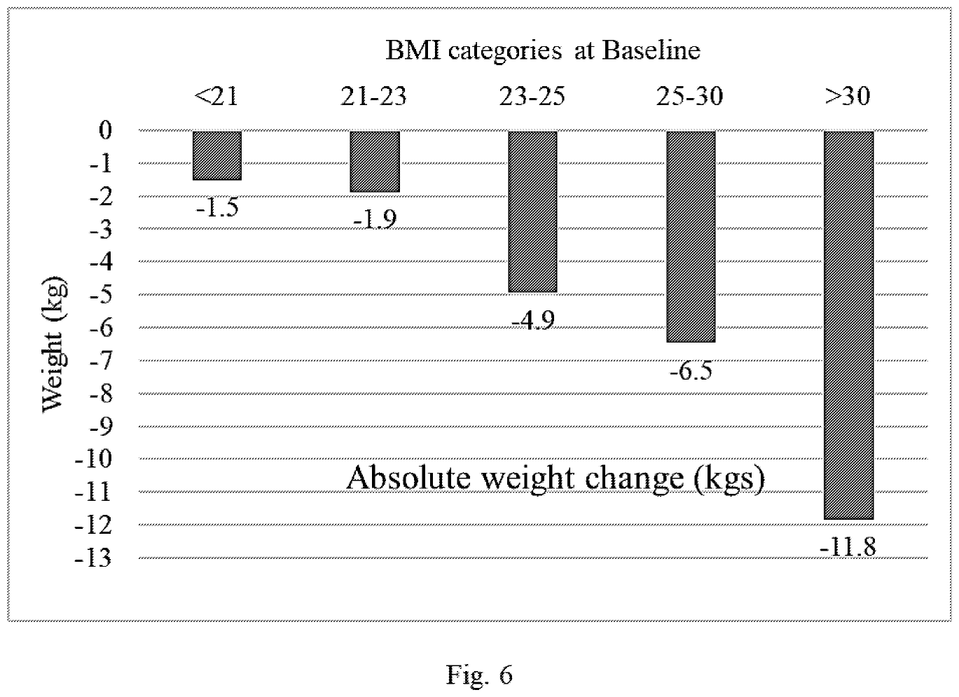

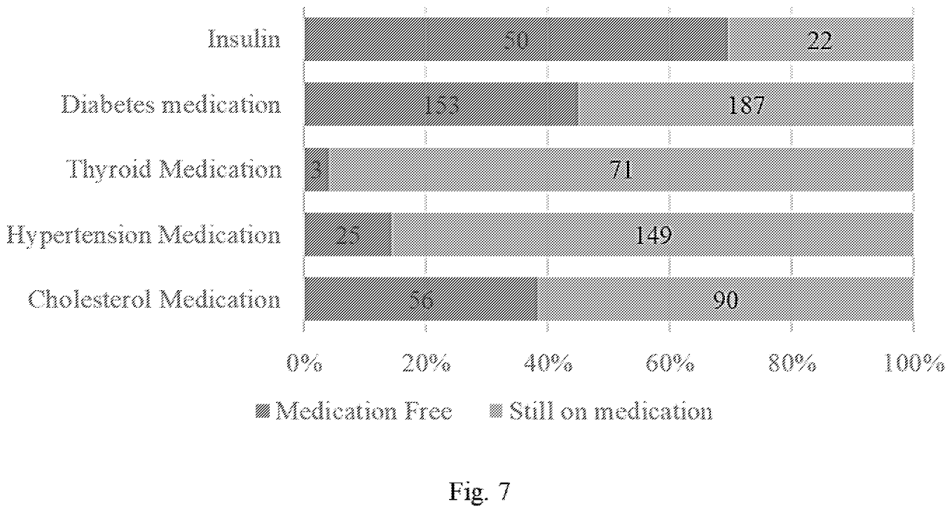

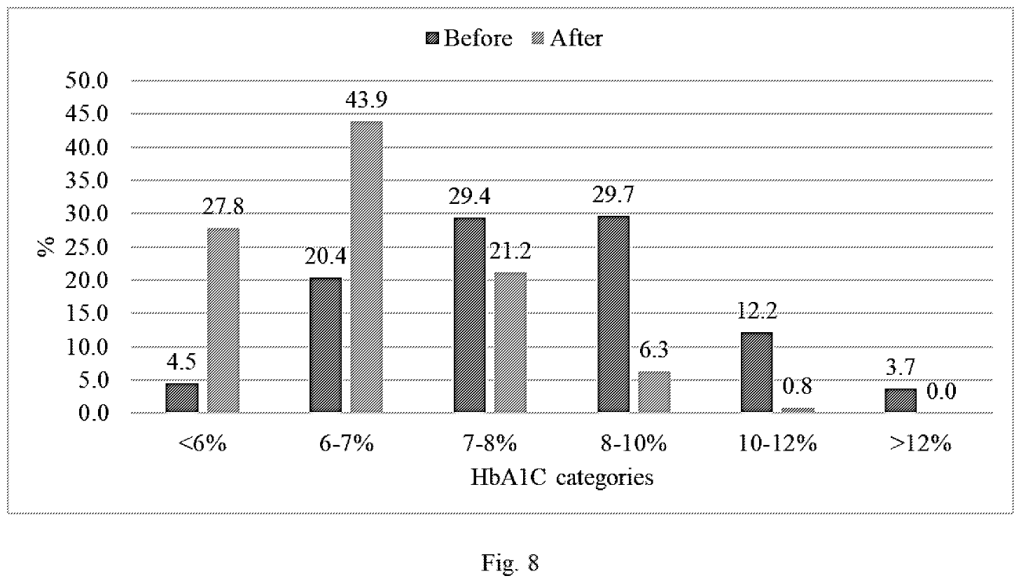

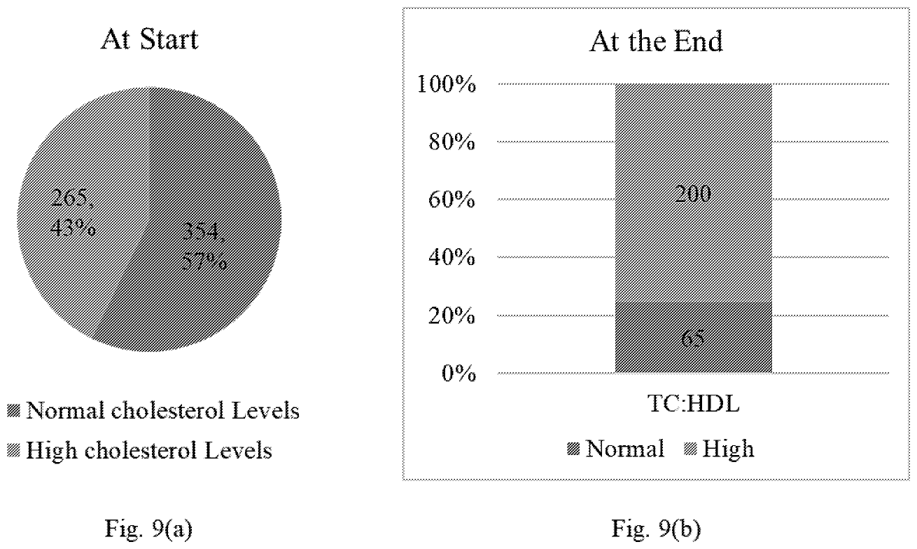

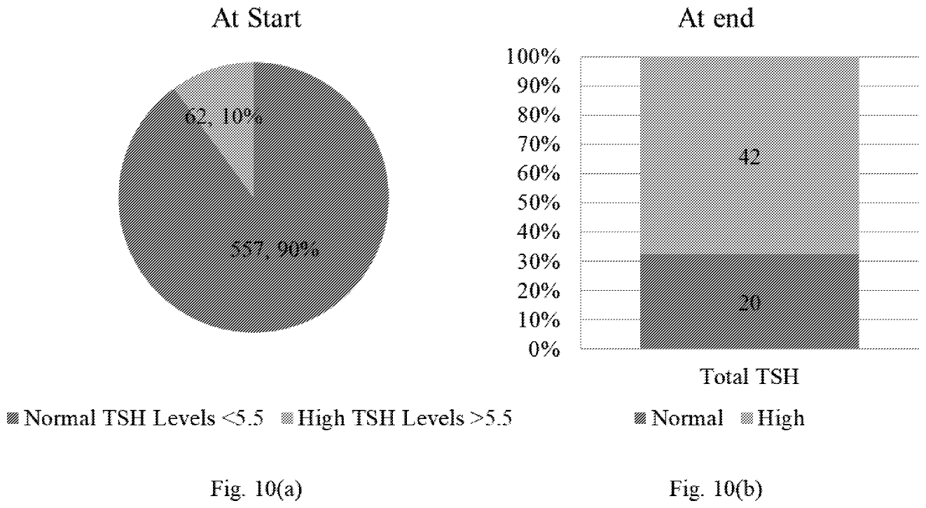

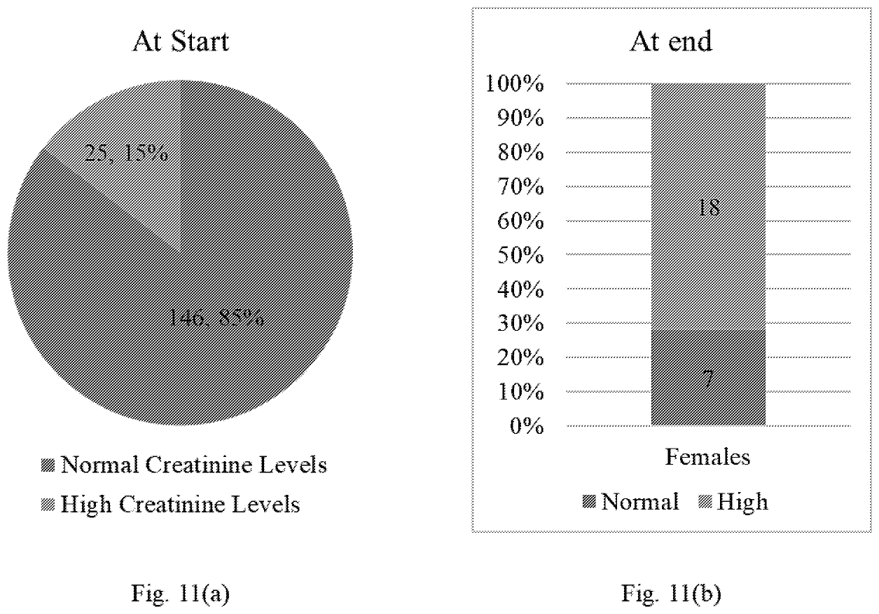

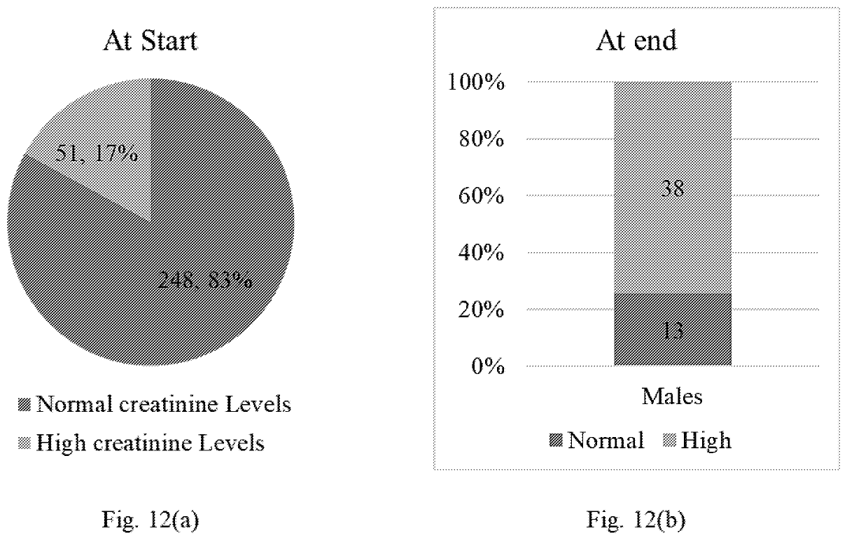

Group 1 consisted of 470 individuals with Type 2 diabetic/prediabetic status. FIG. 4 and Table 13 depict the age distribution among the participants. With 33.3 percent, the age range of 40-50 years had the most number of individuals. Body Mass Index (BMI) of the individual participants was recorded both before and after the intervention program (FIG. 5). FIG. 5 graphically depicts the percentage of Body Mass Index (BMI) of the Type 2 diabetic/prediabetic participants in Group 1, both before and after undergoing the controlled intervention program, according to the embodiments herein. At the start of the program, nearly 41 percent of individuals had a BMI of between 25 and 30, corresponding to the ‘overweight’ category. At the end of the program, only 25 percent of individuals remained in this category. Similarly, the ‘obese’ category, at the start of the program, consisted of 19.2 percent of the 470 individuals, and at the end of the program, the category comprised of only 8.3 percent of individuals. The category of BMI in the range of 21-23 is considered an ‘athletic BMI’, and at the start of the program consisted of 13.7 percent of individuals in Group 1, and at the end of the program, consisted of 23.7 percent of individuals in Group 1. The ‘healthy weight’ category of BMI in the range of 23-25, had 29.8 percent of the individuals at the end of the program, in contrast to only 19.7 percent at the start of the program. The absolute change in the weight is graphically depicted in FIG. 6, where it can be seen that individuals with a BMI of >30 lost on an average 11.8 kilograms of weight after the intervention program. FIG. 6 is a depiction of the absolute change in weight in kilograms, of the Type 2 diabetic/prediabetic participants in Group 1, undergoing the controlled intervention program, according to the embodiments herein. Individuals with BMI of 25-30 lost on average about 6.5 kilograms of weight, those with BMI 23-25 lost on average about 4.9 kilograms of weight, those with BMI 21-23 lost on average 1.9 kilograms and those with BMI<21 lost on average 1.5 kilograms of weight. FIG. 7 graphically represents the number of individuals at the end of program, who are no longer under medication. The percentage of individuals free of the need for medication can be seen in Table 14. Nearly 69 percentage of individuals no longer need insulin medication to manage diabetes, and 38 percent of individuals no longer need cholesterol lowering drugs at the end of the program. A HbA1c test was conducted to see the difference in glycated hemoglobin levels at the start and the end of the intervention program, as depicted in FIG. 8. At the end of the program, it is seen that nearly 44 percent of all individuals showed 6-7 percent of HbA1c, which is considered as good control for a diabetic subject. FIG. 9(a) is a pie chart of the cholesterol levels of individuals in Group 1 of the program at the start. FIG. 9(b) shows the distribution of individuals as a function of cholesterol levels at the end of the program. It is seen that of the 43 percent of individuals who started the program with high cholesterol levels, nearly 20 percent have normal levels of cholesterol at the end of the program. FIG. 10(a) is a pie chart of the Thyroid Stimulating Hormone (TSH) levels of individuals in Group 1 of the program at the start. FIG. 10(b) shows the distribution of individuals as a function of TSH levels at the end of the program. It is seen that of the 10 percent individuals who started the program with high TSH levels, nearly 30 percent have normal levels of TSH at the end of the program. FIG. 11(a) is a pie chart of the creatinine levels of female individuals in Group 1 of the program at the start. FIG. 11(b) shows the distribution of female individuals of Group 1 as a function of creatinine levels at the end of the program. It is seen that of the 15 percent of individuals who started the program with high creatinine levels, nearly 25 percent had normal levels of creatinine at the end of the program. Similarly, FIG. 12(a) is a pie chart of the creatinine levels of male individuals in Group 1 of the program at the start. FIG. 12(b) shows the distribution of male individuals as a function of creatinine levels at the end of the program. It is seen that of the 17 percent of individuals who started the program with high creatinine levels, nearly 21 percent have normal levels of creatinine at the end of the program.

TABLE 13
Age Distribution in participants in Group 1

| Parameter | Number |
|---|---|
| Total no. of active paparticipants | 470 |
| No. of males | 299 |
| No. of females | 171 |
| Primary diabetics | 419 |
| Weight loss | 20 |
| Supporting | 31 |

TABLE 14
Percentage of freedom from medication for participants in Group 1

| Parameter | Number |
|---|---|
| Insulin | 69.4 |
| Diabetes medication | 45 |
| Thyroid Medication | 4.1 |
| Hypertension Medication | 14.4 |
| Cholesterol Medication | 38.4 |

### Group 2

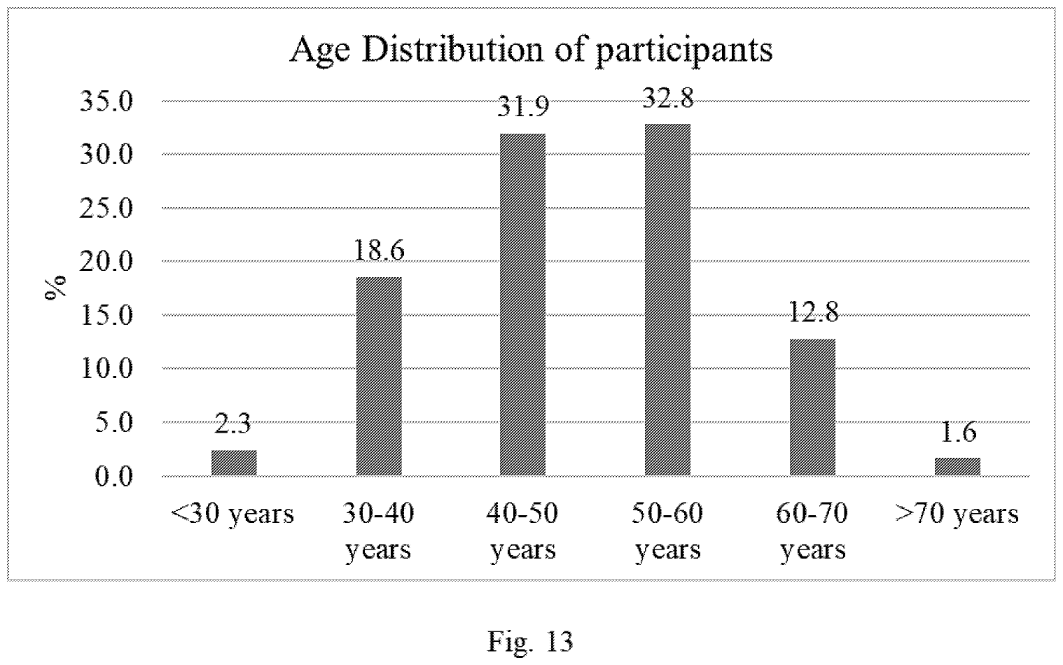

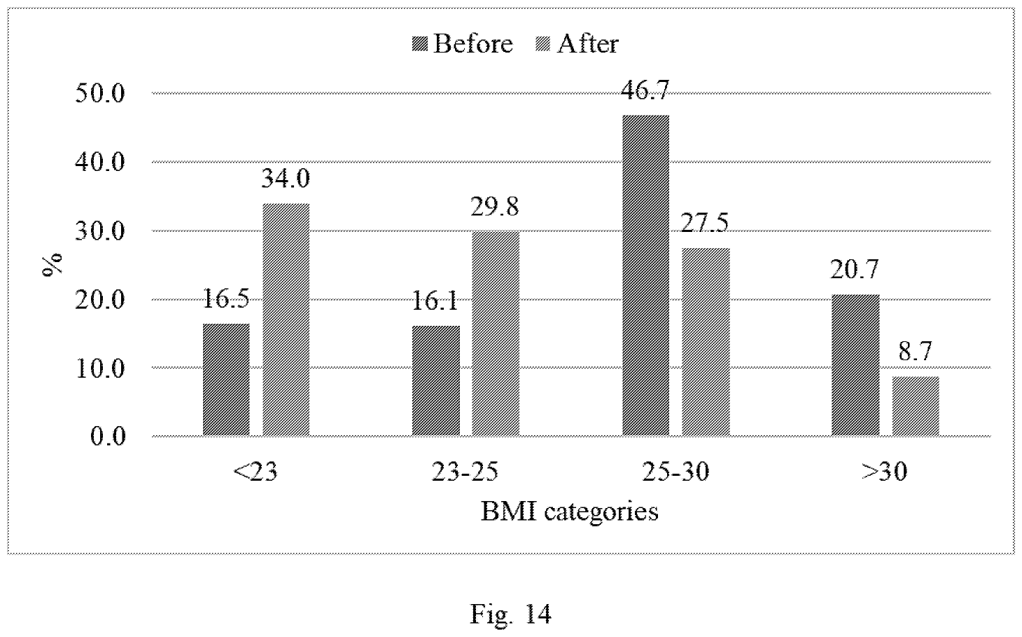

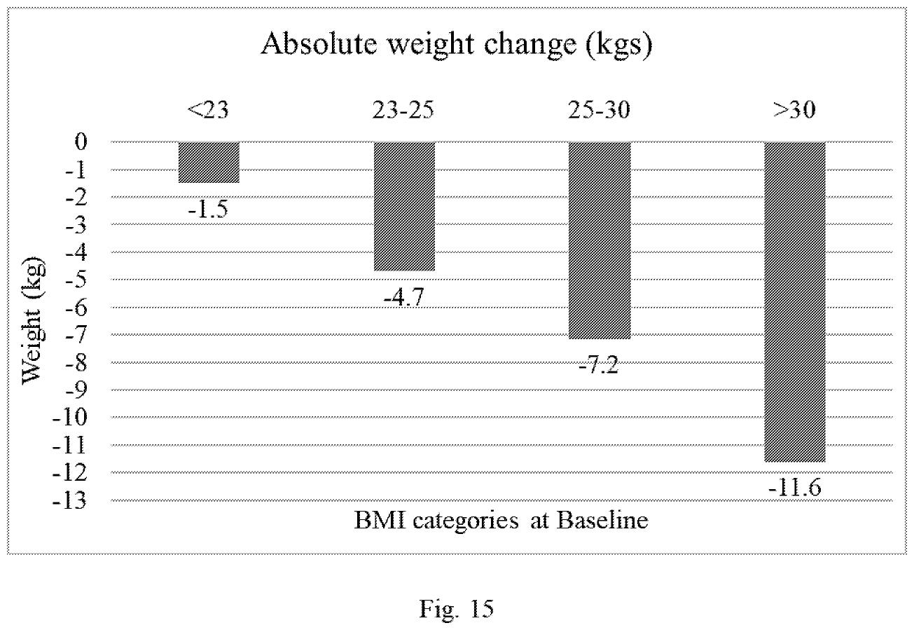

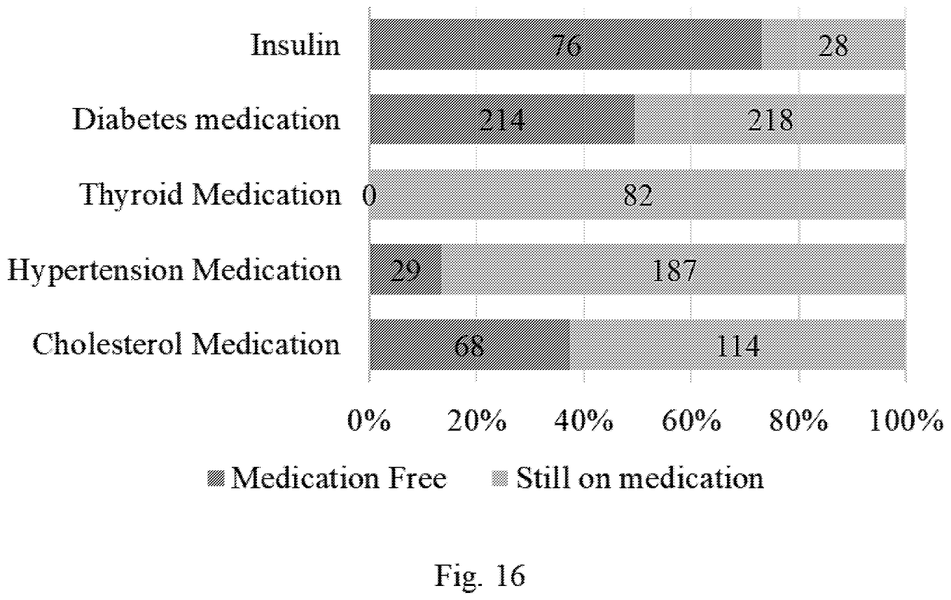

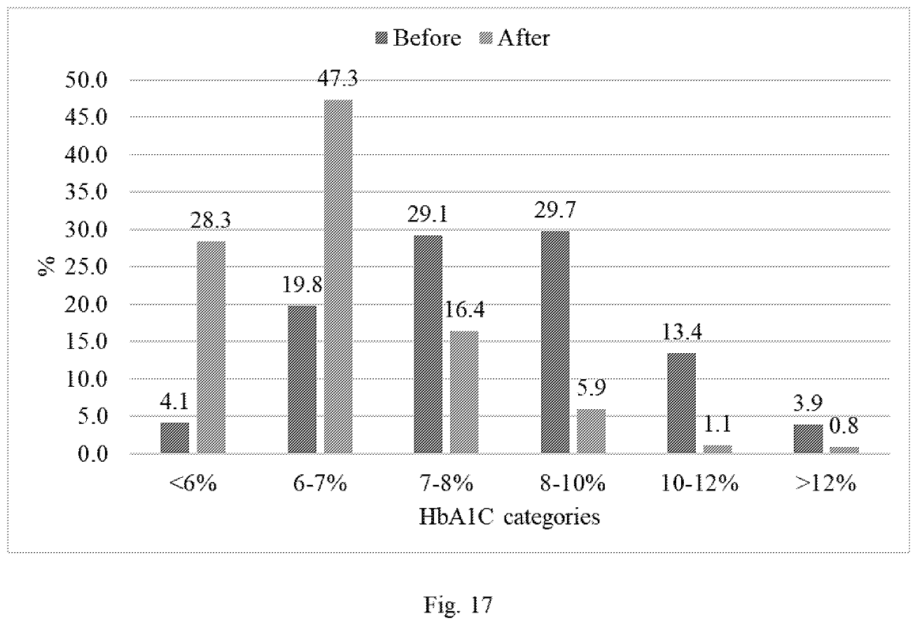

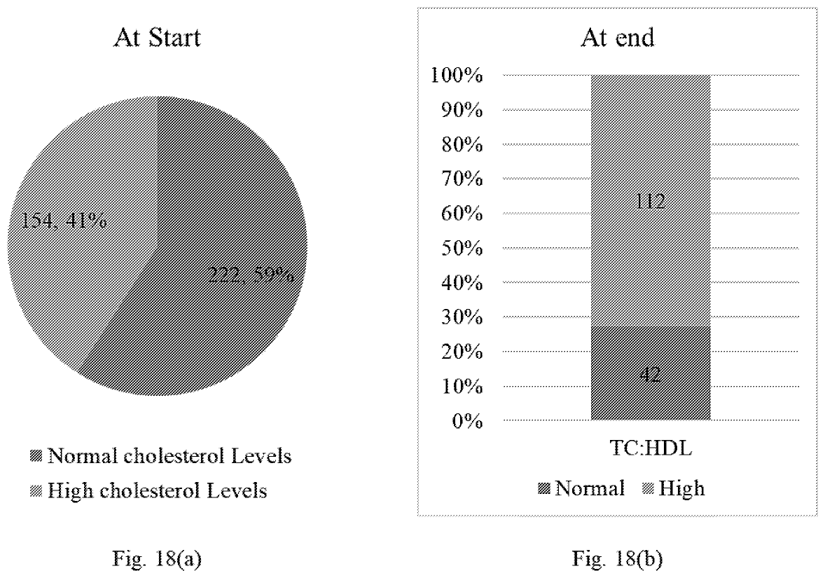

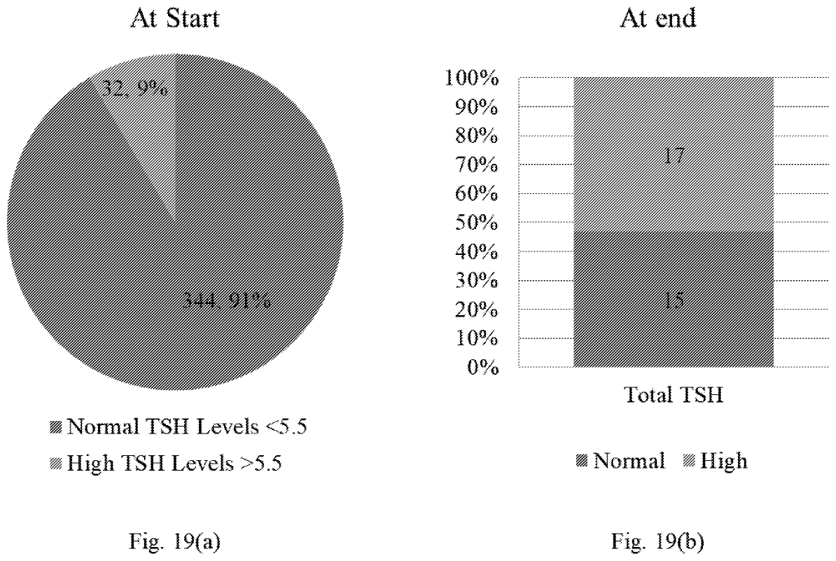

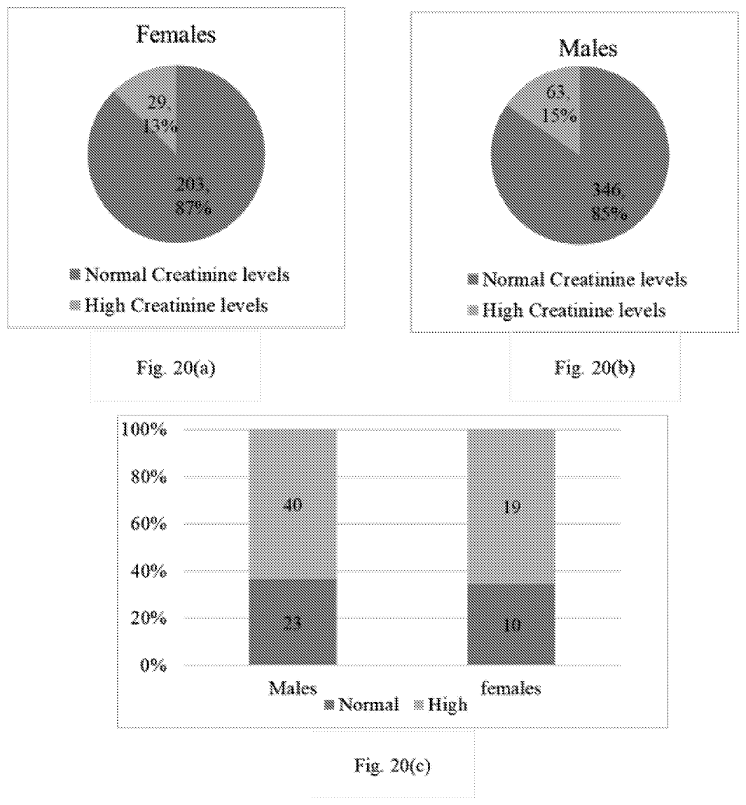

Group 2 consisted of 643 individuals with Type 2 diabetic/prediabetic status. FIG. 13 and Table 15 depict the age distribution among the participants. It was seen that with 32.8 percent, the age range of 50-60 years had the most number of individuals. Body Mass Index (BMI) of the individual participants was recorded both before and after the intervention program (FIG. 14). At the start of the program, nearly 46.7 percent of individuals had a BMI of between 25 and 30, corresponding to the ‘overweight’ category. At the end of the program, only 27.5 percent of individuals remained in this category. Similarly, the ‘obese’ category, at the start of the program, consisted of 20.7 percent of the 643 individuals, and at the end of the program, the category comprised of only 8.7 percent of individuals. The ‘healthy weight’ categories of BMI in the range of 23-25 had 29.8 percent of the individuals, at the end of the program in contrast to only 16.1 percent at the start of the program. The absolute change in the weight is graphically depicted in FIG. 15, where it can be seen that individuals with a BMI of >30 on an average lost 11.6 kilograms of weight after the intervention program. Individuals with BMI of 25-30 on an average, lost about 6.5 kilograms of weight, those with BMI 23-25 on an average, lost about 7.2 kilograms of weight, those with BMI<21 on an average, lost 1.5 kilograms of weight. FIG. 16 graphically represents the number of individuals at the end of program, who are no longer under medication. The percentage of individuals free of the need for medication can be seen in Table 16. Nearly 73 percentage of individuals no longer need insulin medication to manage diabetes, and 49 percent of individuals no longer need diabetes medication at the end of the program. A HbA1c test was conducted to see the difference in glycated hemoglobin levels at the start and the end of the intervention program, as depicted in FIG. 17. At the end of the program, it is seen that nearly 47 percent of all individuals showed 6-7 percent of HbA1c, which is considered as good control for a diabetic subject. FIG. 18(a) is a pie chart of the cholesterol levels of individuals in Group 2 of the program at the start. FIG. 18(b) shows the distribution of individuals as a function of cholesterol levels at the end of the program. It is seen that of the 41 percent of individuals who started the program with high cholesterol levels, nearly 27 percent has normal levels of cholesterol at the end of the program. FIG. 19(a) is a pie chart of the Thyroid Stimulating Hormone (TSH) levels of individuals in Group 2 of the program at the start. FIG. 19(b) shows the distribution of individuals as a function of TSH levels at the end of the program. It is seen that of the 9 percent of individuals who started the program with high TSH levels, nearly 45 percent have normal levels of TSH at the end of the program. FIG. 20(a) is a pie chart of the creatinine levels of female individuals in Group 2 of the program at the start. FIG. 20(b) is a pie chart of the creatinine levels of male individuals in Group 2 of the program at the start. FIG. 20(c) shows the distribution of female individuals of Group 2 as a function of creatinine levels at the end of the program. It is seen that of the 13 and 15 percent of female and male individuals who started the program with high creatinine levels, nearly 35 and 30 percent had normal levels of creatinine at the end of the program, respectively.

TABLE 15
Age Distribution in participants in Group 2

| Parameter | Number |
|---|---|
| Total no. of active participants | 643 |
| No. of males | 378 |
| No. of females | 265 |
| Primary diabetics | 531 |
| Weight loss | 24 |
| Supporting | 88 |

TABLE 16
Percentage of freedom from medication for participants in Group 2

| Parameter | Number |
|---|---|
| Insulin | 73.1 |
| Diabetes medication | 49.5 |
| Thyroid Medication | 0 |
| Hypertension Medication | 13.4 |
| Cholesterol Medication | 37.4 |

### Group 3

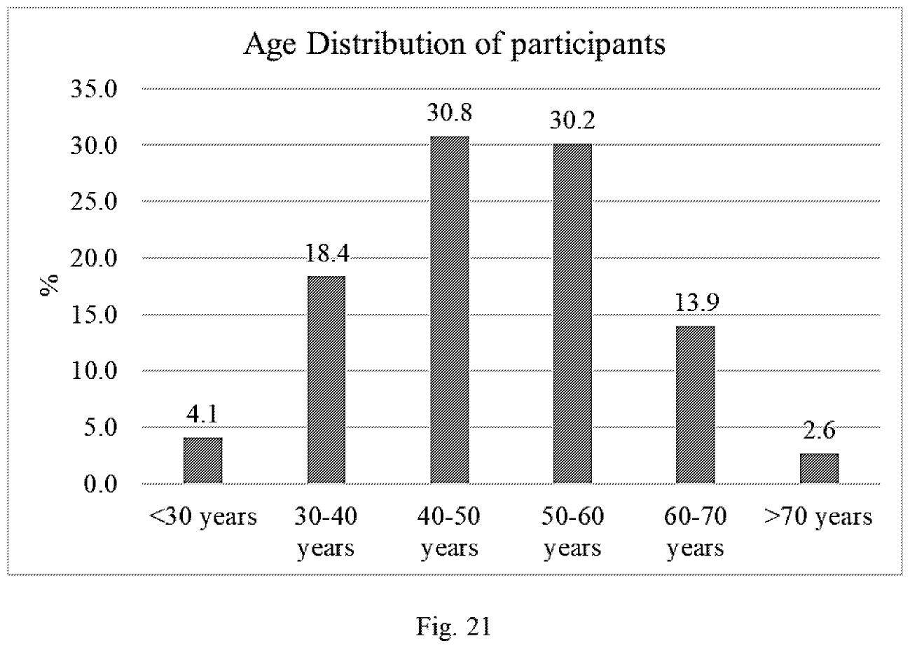

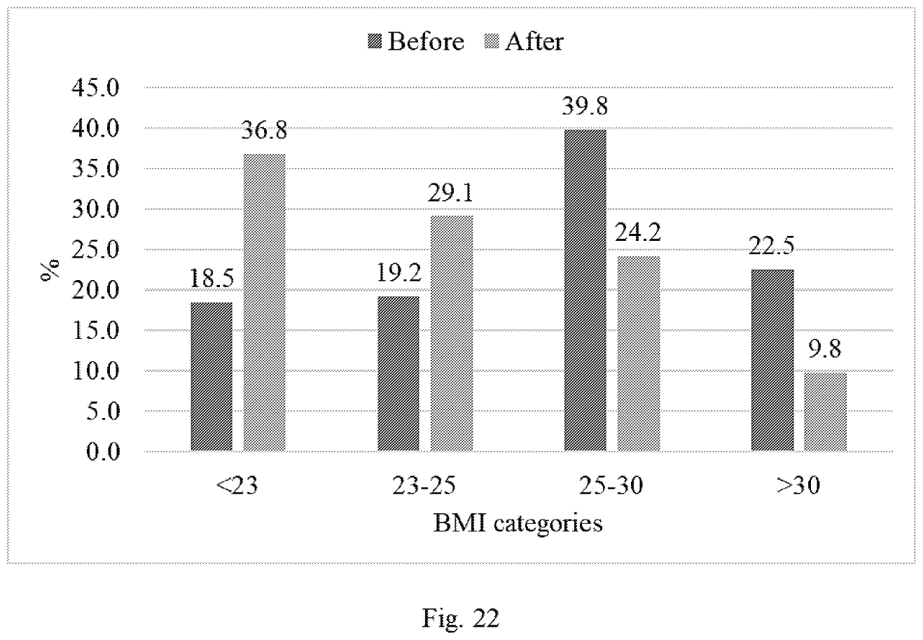

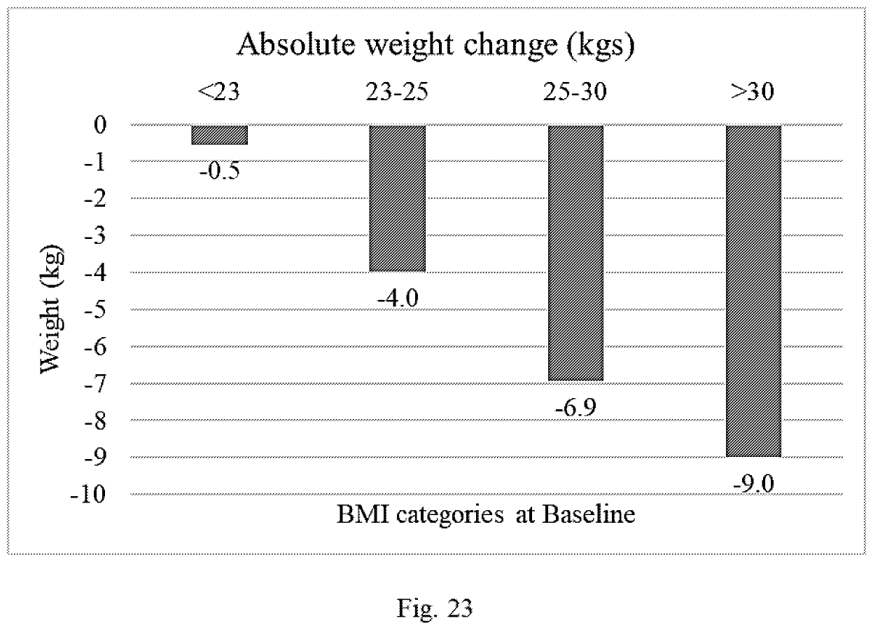

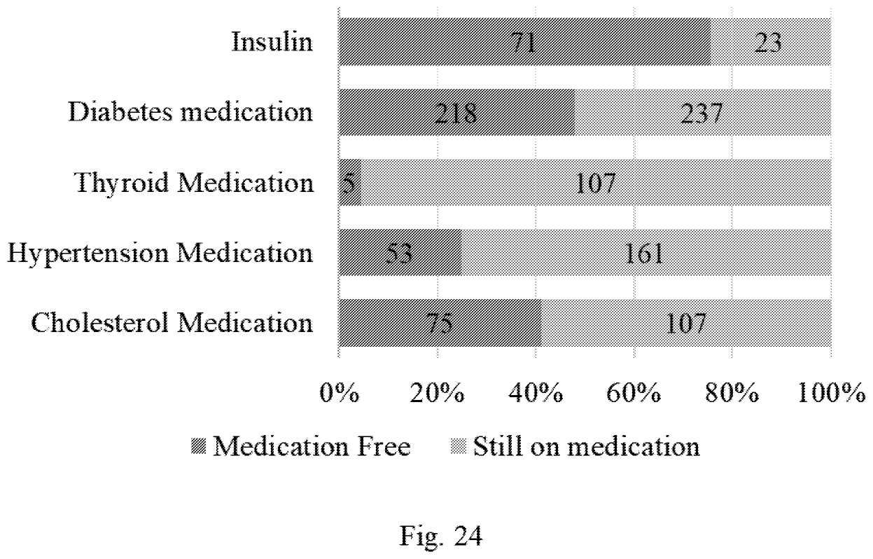

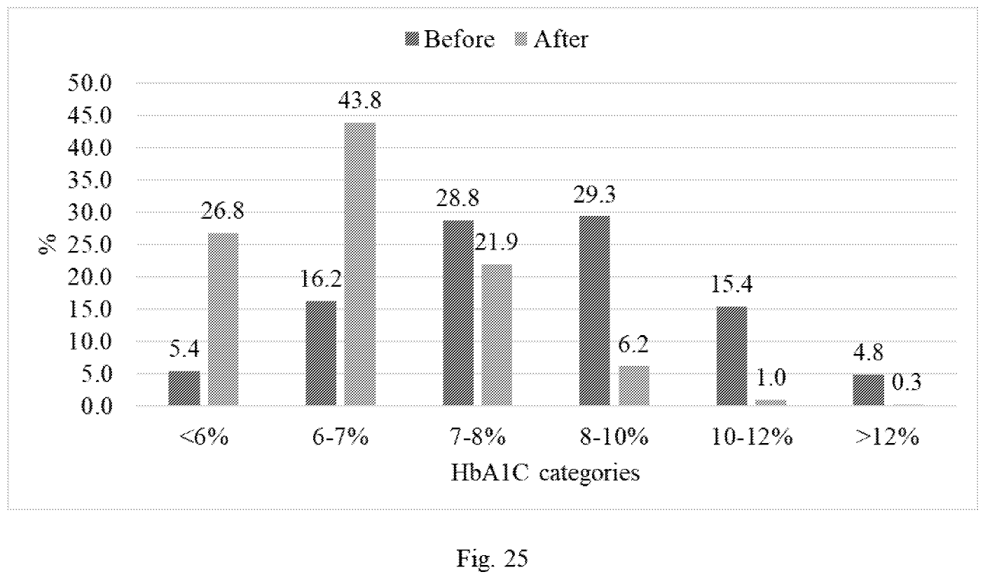

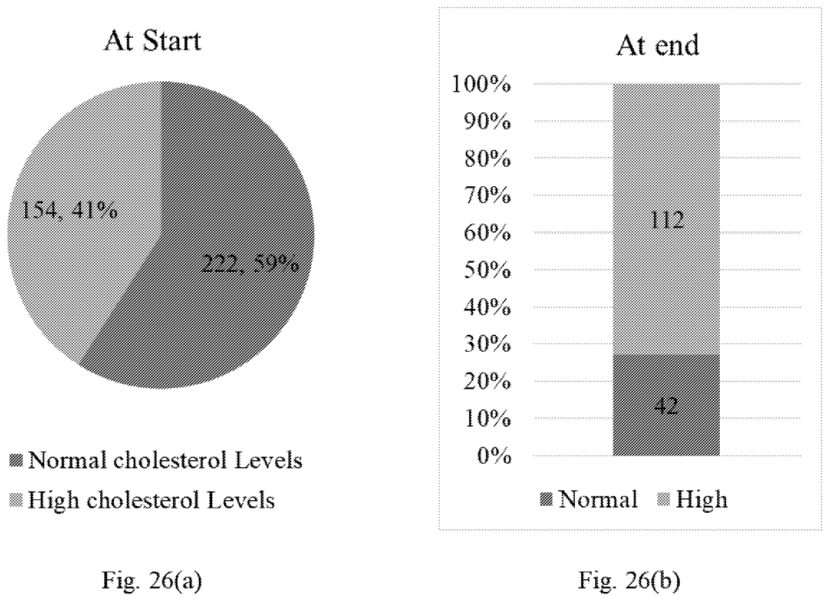

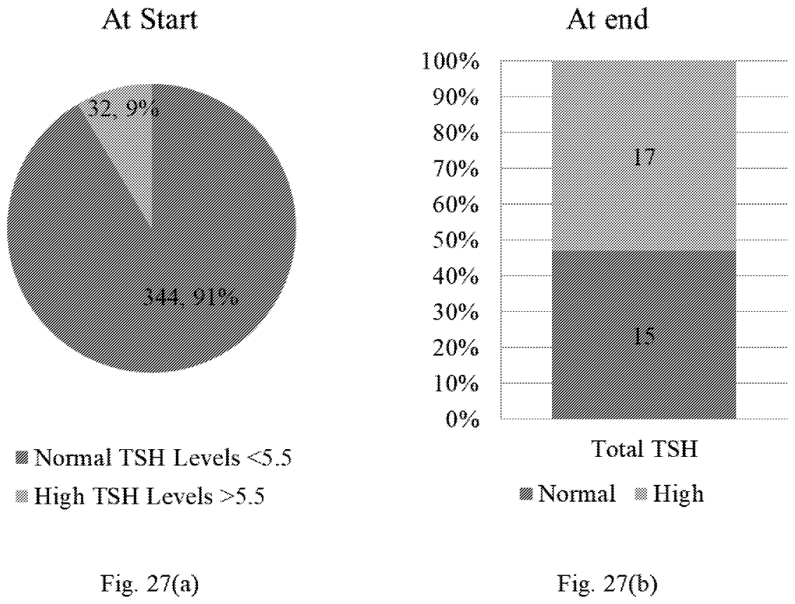

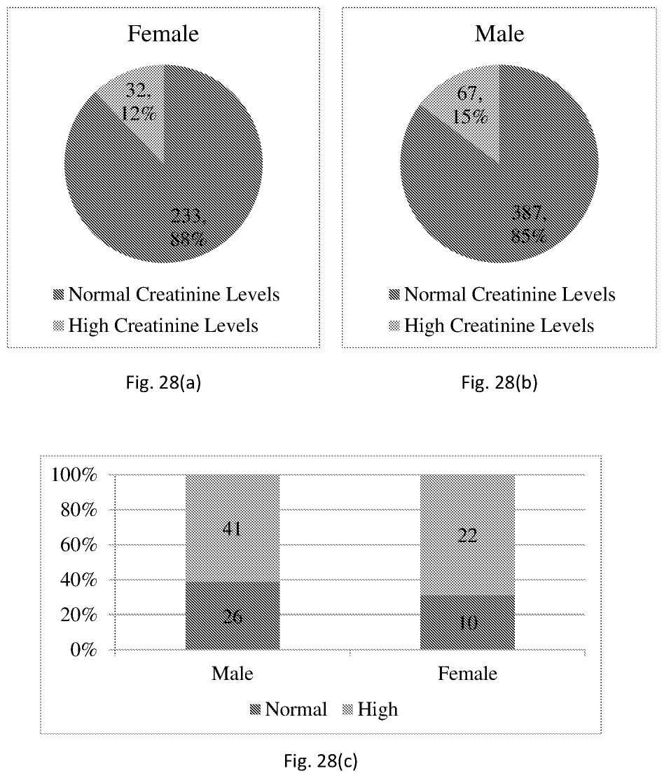

Group 3 consisted of 711 individuals with Type 2 diabetic/prediabetic status. FIG. 21 and Table 17 depict the age distribution among the participants. It was seen that with 30.8 percent, the age range of 40-50 years had the most number of individuals. Body Mass Index (BMI) of the individual participants was recorded both before and after the intervention program (FIG. 22). At the start of the program, nearly 39.8 percent of individuals had a BMI of between 25 and 30, corresponding to the ‘overweight’ category. At the end of the program, only 24.2 percent of individuals remained in this category. Similarly, the ‘obese’ category, at the start of the program, consisted of 22.5 percent of the 711 individuals, and at the end of the program, the category comprised of only 9.8 percent of individuals. The ‘healthy weight’ categories of BMI in the range of 23-25 had 29.1 percent of the individuals, at the end of the program in contrast to only 19.2 percent at the start of the program. The absolute change in the weight is graphically depicted in FIG. 23, where it can be seen that individuals with a BMI of >30, on an average, lost 9.0 kilograms of weight after the intervention program. Individuals with BMI of 25-30, on an average, lost about 6.9 kilograms of weight, those with BMI 23-25, on an average, lost about 4.0 kilograms of weight, those with BMI<21, on an average, lost 0.5 kilograms of weight. FIG. 24 graphically represents the number of individuals at the end of program, who are no longer under medication. The percentage of individuals free of the need for medication can be seen in Table 18. Nearly 75 percentage of individuals no longer need insulin medication to manage diabetes, and 48 percent of individuals no longer need diabetes medication at the end of the program. A HbA1c test was conducted to see the difference in glycated hemoglobin levels at the start and the end of the intervention program, as depicted in FIG. 25. At the end of the program, it is seen that nearly 44 percent of all individuals showed 6-7 percent of HbA1c, which is considered as good control for a diabetic subject. FIG. 26(a) is a pie chart of the cholesterol levels of individuals in Group 3 of the program at the start. FIG. 26(b) shows the distribution of individuals as a function of cholesterol levels at the end of the program. It is seen that of the 41 percent of individuals who started the program with high cholesterol levels, nearly 27 percent have normal levels of cholesterol at the end of the program. FIG. 27(a) is a pie chart of the Thyroid Stimulating Hormone (TSH) levels of individuals in Group 3 of the program at the start. FIG. 27(b) shows the distribution of individuals as a function of TSH levels at the end of the program. It is seen that of the 9 percent of individuals who started the program with high TSH levels, nearly 45 percent have normal levels of TSH at the end of the program. FIG. 28(a) is a pie chart of the creatinine levels of female individuals in Group 3 of the program at the start. FIG. 28(b) is a pie chart of the creatinine levels of male individuals in Group 3 of the program at the start. FIG. 28(c) shows the distribution of female individuals of Group 3 as a function of creatinine levels at the end of the program. It is seen that of the 12 and 15 percent of female and male individuals who started the program with high creatinine levels, nearly 39 and 31 percent had normal levels of creatinine at the end of the program, respectively.

TABLE 17
Age Distribution in participants in Group 3

| Parameter | Number |
|---|---|
| Total no. of active participants | 711 |
| No. of males | 422 |
| No. of females | 289 |
| Primary diabetics | 587 |
| Weight loss | 46 |
| Supporting | 78 |

TABLE 18
Percentage of freedom from medication for participants in Group 3

| Parameter | Number |
|---|---|
| Insulin | 75.5 |
| Diabetes medication | 47.9 |
| Thyroid Medication | 4.5 |
| Hypertension Medication | 24.8 |
| Cholesterol Medication | 41.2 |

### Group 4

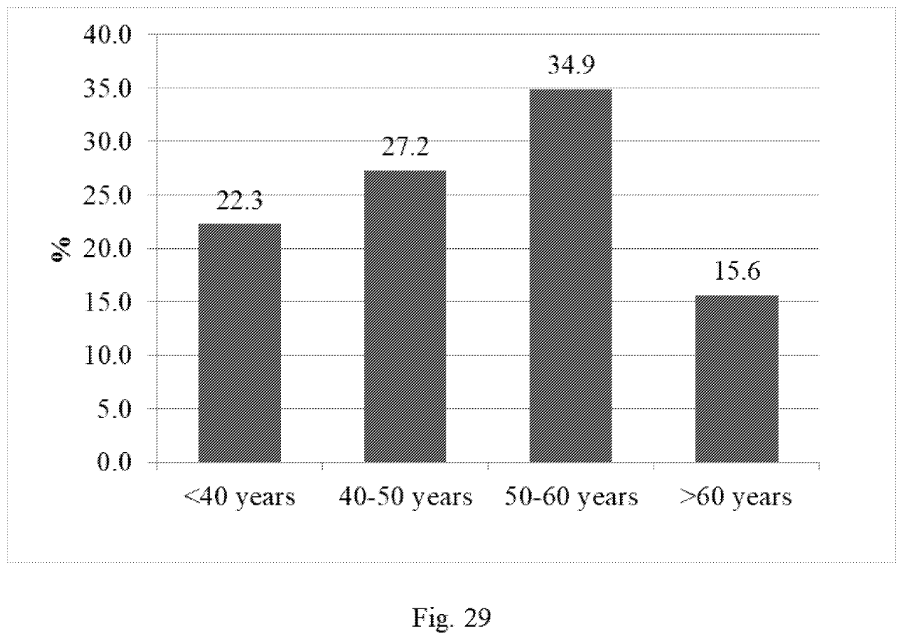

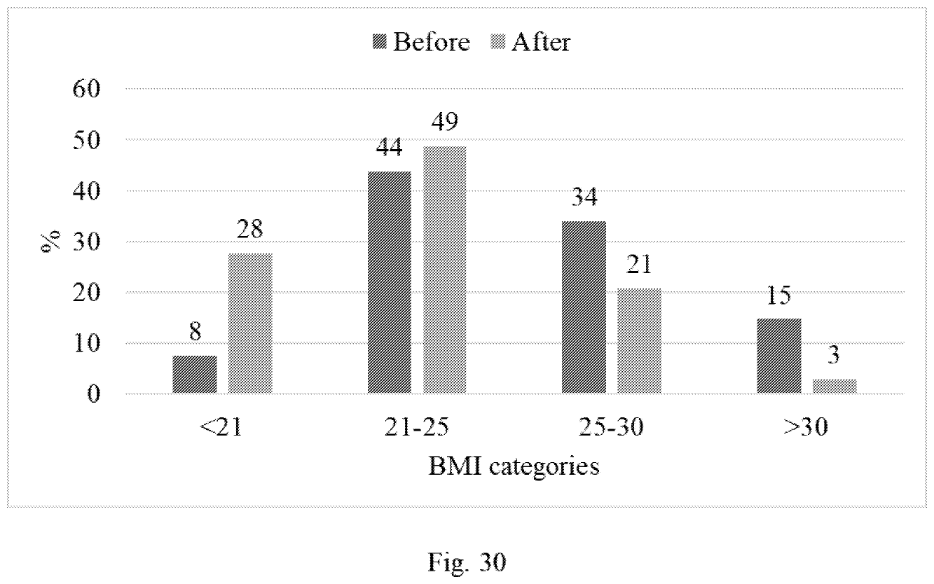

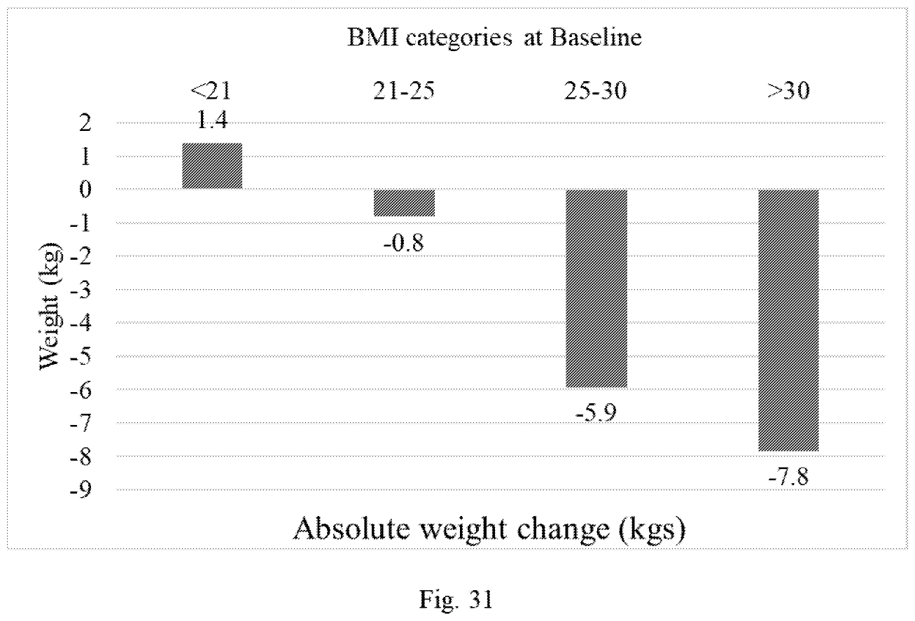

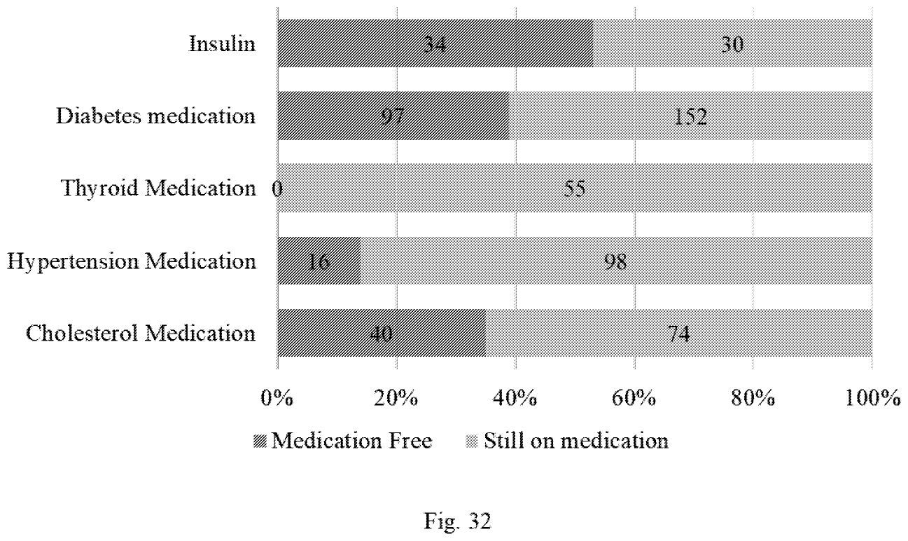

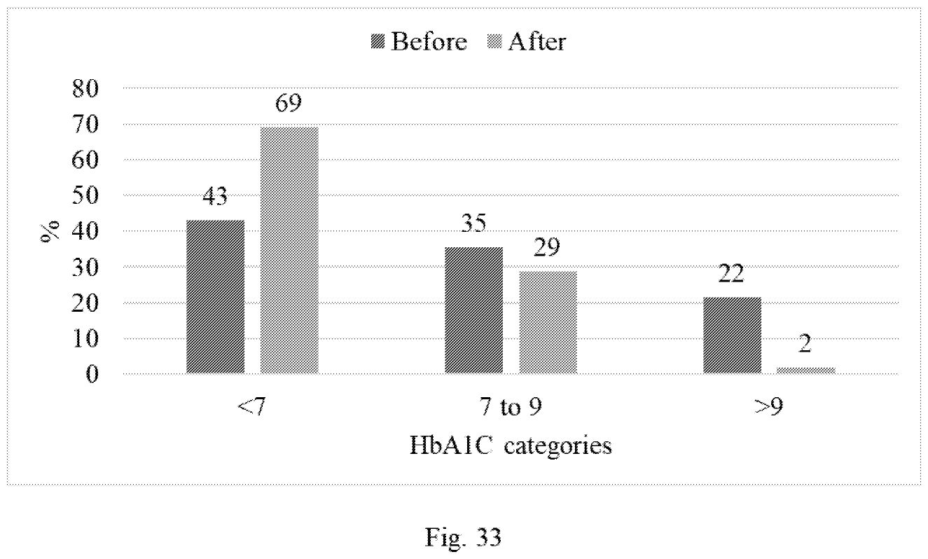

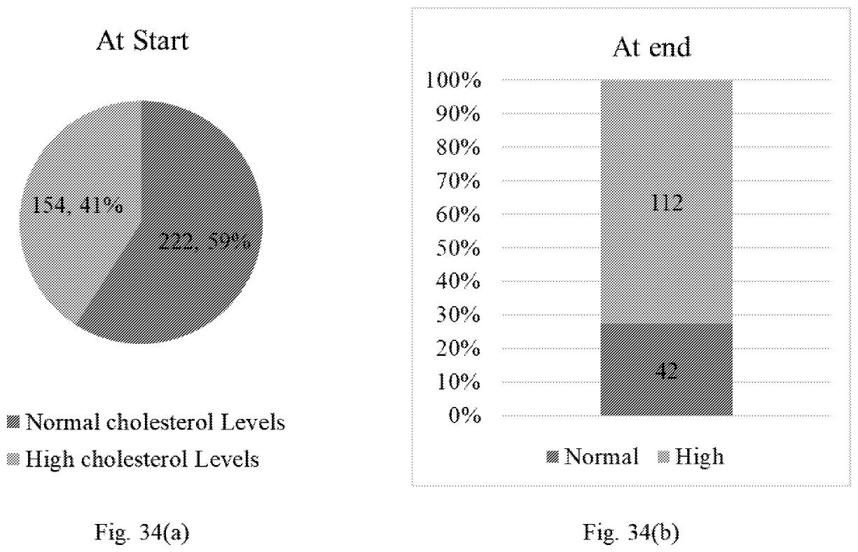

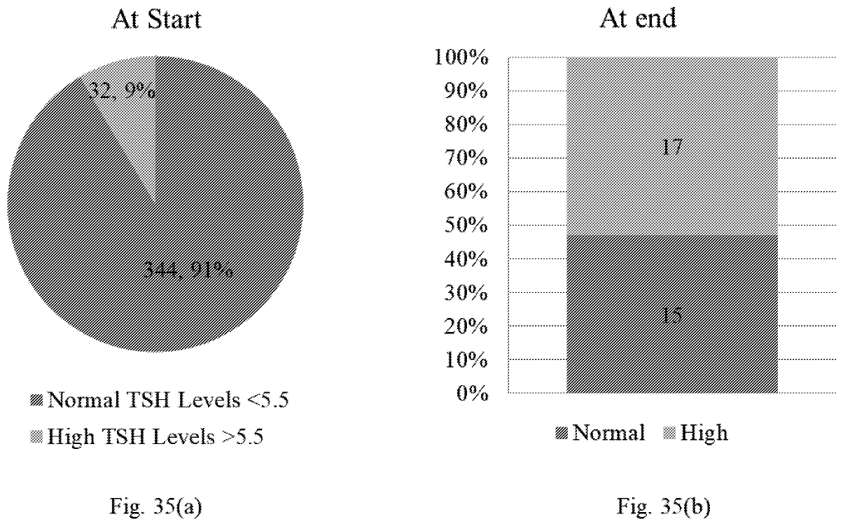

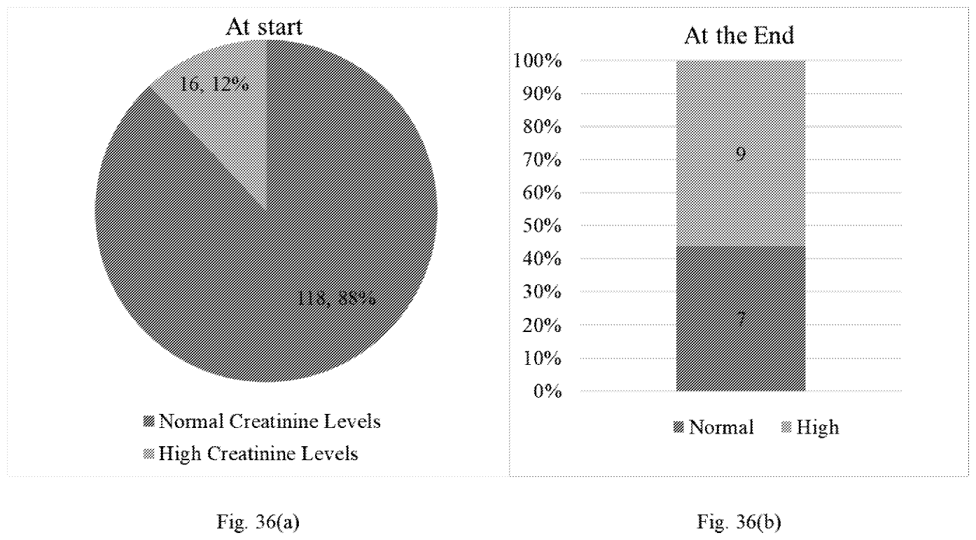

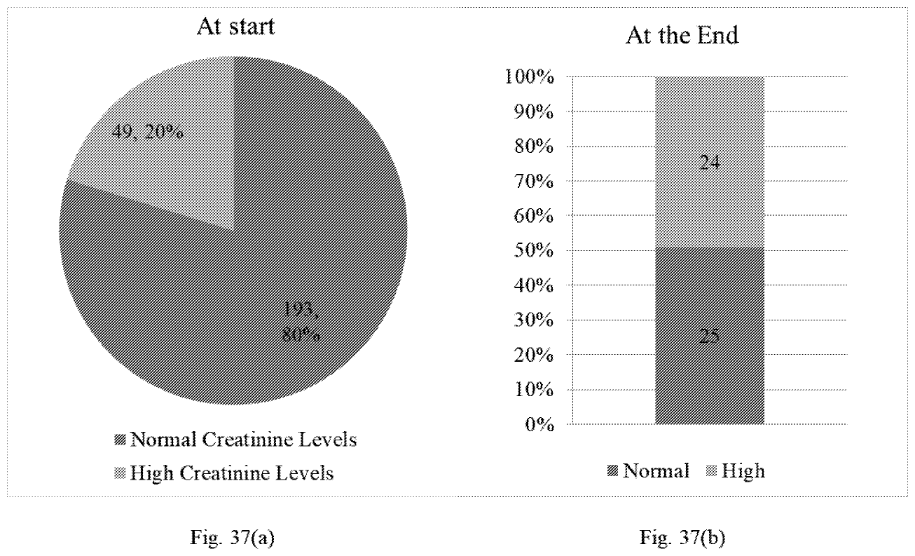

Group 4 consisted of 376 individuals with Type 2 diabetic/prediabetic status. FIG. 29 and Table 19 depict the age distribution among the participants. It was seen that with 34.9 percent, the age range of 50-60 years had the most number of individuals. Body Mass Index (BMI) of the individual participants was recorded both before and after the intervention program (FIG. 30). At the start of the program, nearly 34 percent of individuals had a BMI of between 25 and 30, corresponding to the ‘overweight’ category. At the end of the program, only 21 percent of individuals remained in this category. Similarly, the ‘obese’ category, at the start of the program, consisted of 15 percent of the 376 individuals, and at the end of the program, the category comprised of only 3 percent of individuals. The ‘healthy weight’ categories of BMI in the range of 21-25 had 20 nearly 49 percent of the individuals, at the end of the program in contrast to 44 percent at the start of the program. The absolute change in the weight is graphically depicted in FIG. 31, where it can be seen that individuals with a BMI of >30 on an average lost 7.8 kilograms of weight after the intervention program. Individuals with BMI of 25-30 on an average lost about 5.9 kilograms of weight, those with BMI 21-25 on an average lost about 0.8 kilograms of weight, those with BMI<21 on an average gained 1.4 kilograms of weight. FIG. 32 graphically represents the number of individuals at the end of program, who are no longer under medication. The percentage of individuals free of the need for medication can be seen in Table 20. Nearly 53 percentage of individuals no longer need insulin medication to manage diabetes, and 39 percent of individuals no longer need diabetes medication at the end of the program. A HbA1c test was conducted to see the difference in glycated hemoglobin levels at the start and the end of the intervention program, as depicted in FIG. 33. At the end of the program, it is seen that nearly 69 percent of all individuals showed<7 percent of HbA1c, which is considered as good control for a diabetic subject. FIG. 34(a) is a pie chart of the cholesterol levels of individuals in Group 4 of the program at the start. FIG. 34(b) shows the distribution of individuals as a function of cholesterol levels at the end of the program. It is seen that of the 41 percent of individuals who started the program with high cholesterol levels, nearly 27 percent have normal levels of cholesterol at the end of the program. FIG. 35(a) is a pie chart of the Thyroid Stimulating Hormone (TSH) levels of individuals in Group 4 of the program at the start. FIG. 35(b) shows the distribution of individuals as a function of TSH levels at the end of the program. It is seen that of the 9 percent of individuals who started the program with high TSH levels, nearly 47 percent have normal levels of TSH at the end of the program. FIG. 36(a) is a pie chart of the creatinine levels of female individuals in Group 4 of the program at the start. FIG. 36(b) shows the distribution of female individuals of Group 4 as a function of creatinine levels at the end of the program. It is seen that of the 12 percent of individuals who started the program with high creatinine levels, nearly 44 percent had normal levels of creatinine at the end of the program. Similarly, FIG. 37(a) is a pie chart of the creatinine levels of male individuals in Group 4 of the program at the start. FIG. 37(b) shows the distribution of male individuals as a function of creatinine levels at the end of the program. It is seen that of the percent of individuals who started the program with high creatinine levels, nearly 50 percent has normal levels of creatinine at the end of the program.

TABLE 19
Age Distribution in participants in Group 4

| Parameter | Number |
|---|---|
| Total no. of active participants | 376 |
| Primary diabetics | 306 |
| Weight loss | 38 |
| Supporting | 32 |

TABLE 20
Percentage of freedom from medication for participants in Group 4

| Parameter | Number |
|---|---|
| Insulin | 53.1 |
| Diabetes medication | 39 |
| Thyroid Medication | 0 |
| Hypertension Medication | 14 |
| Cholesterol Medication | 35.1 |

### Group 5

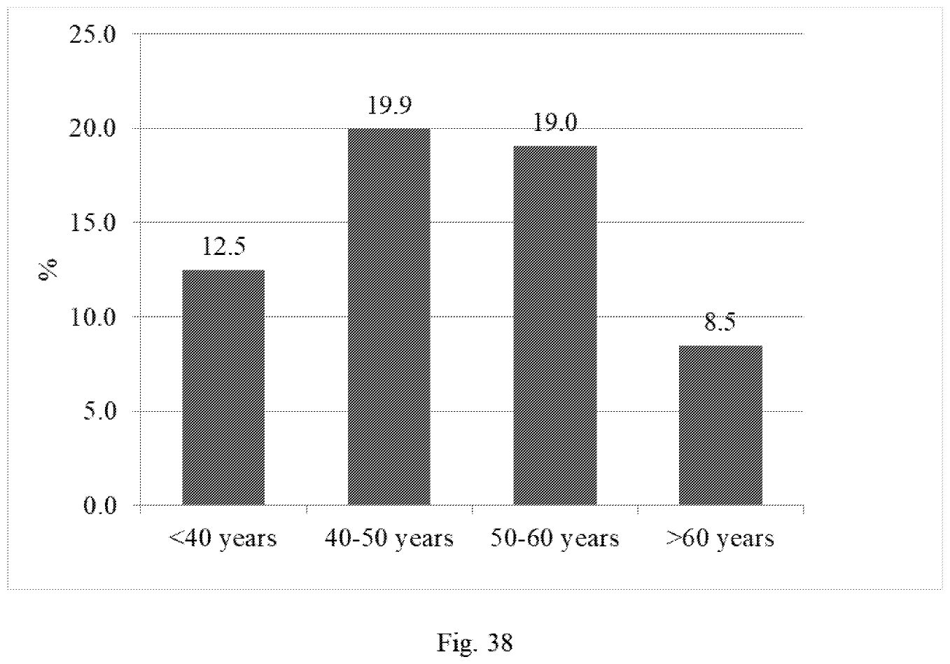

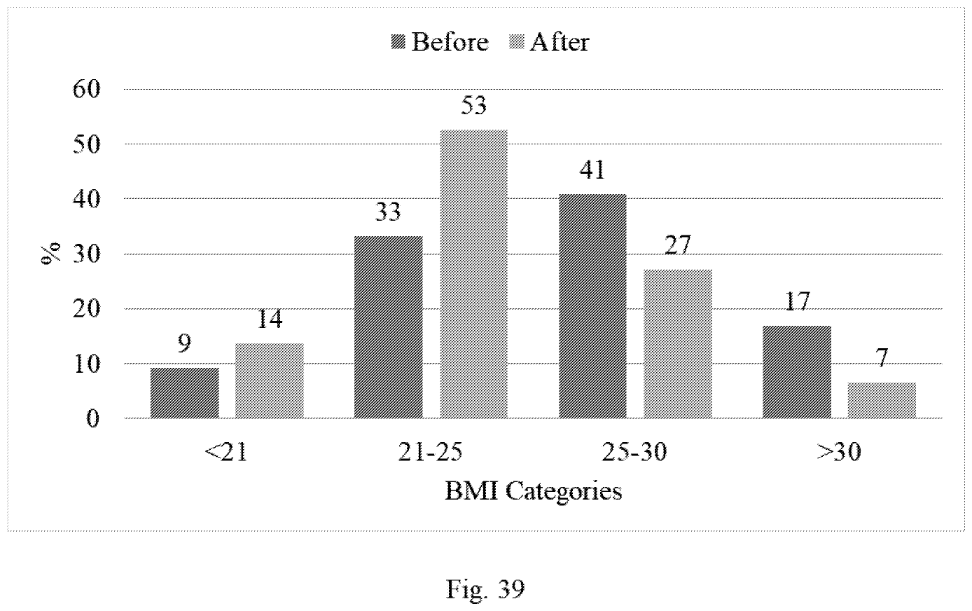

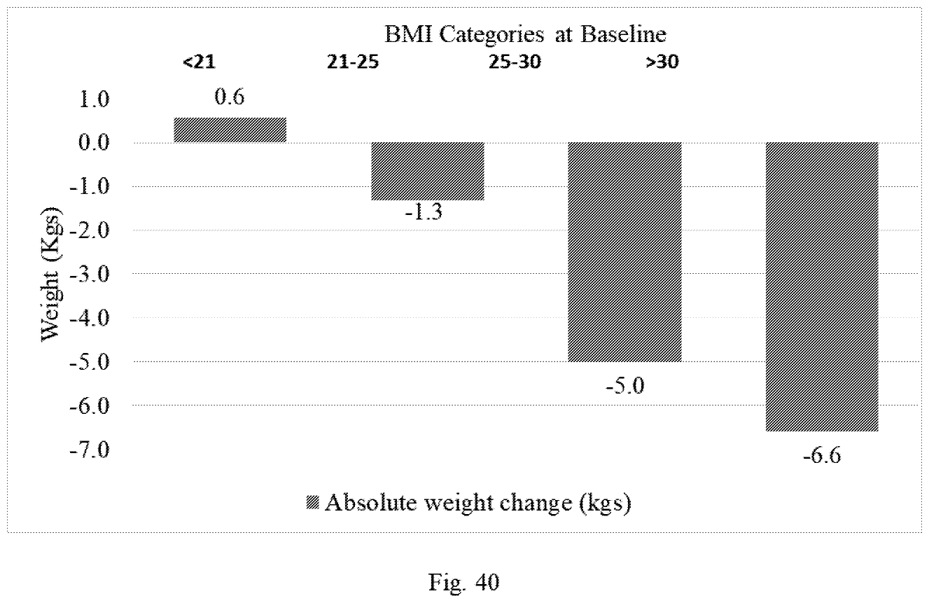

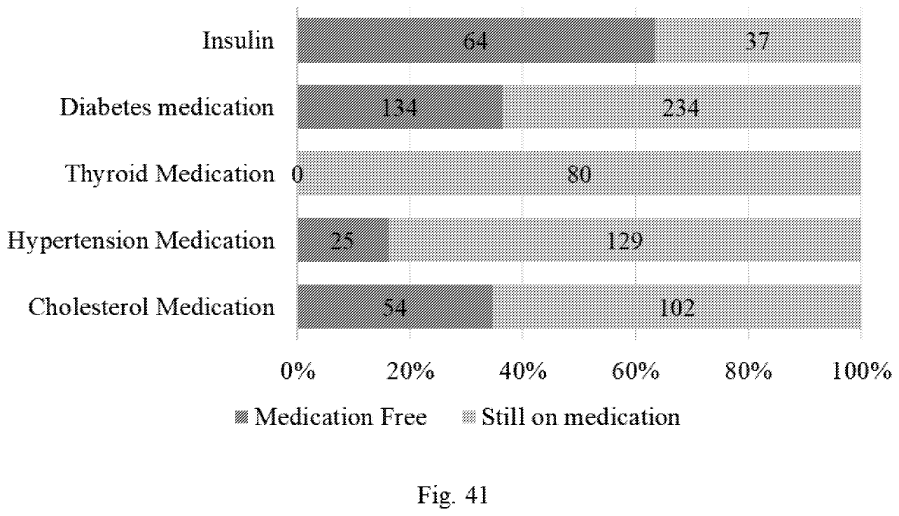

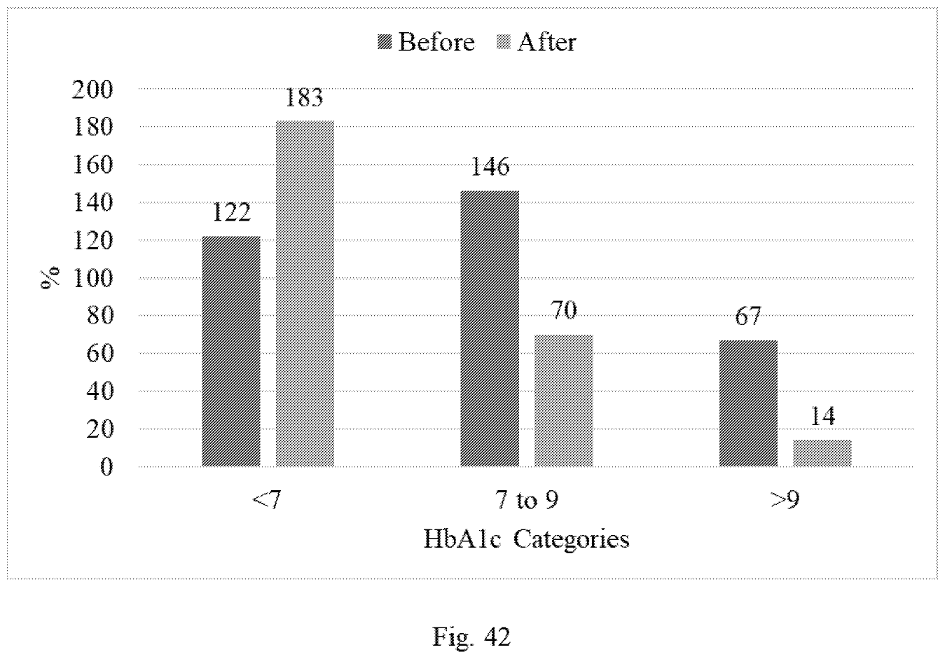

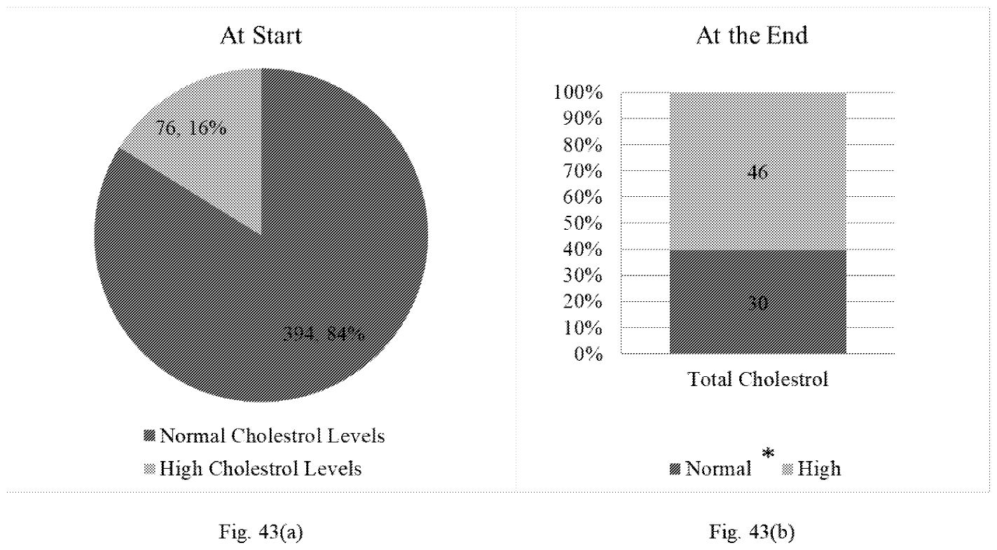

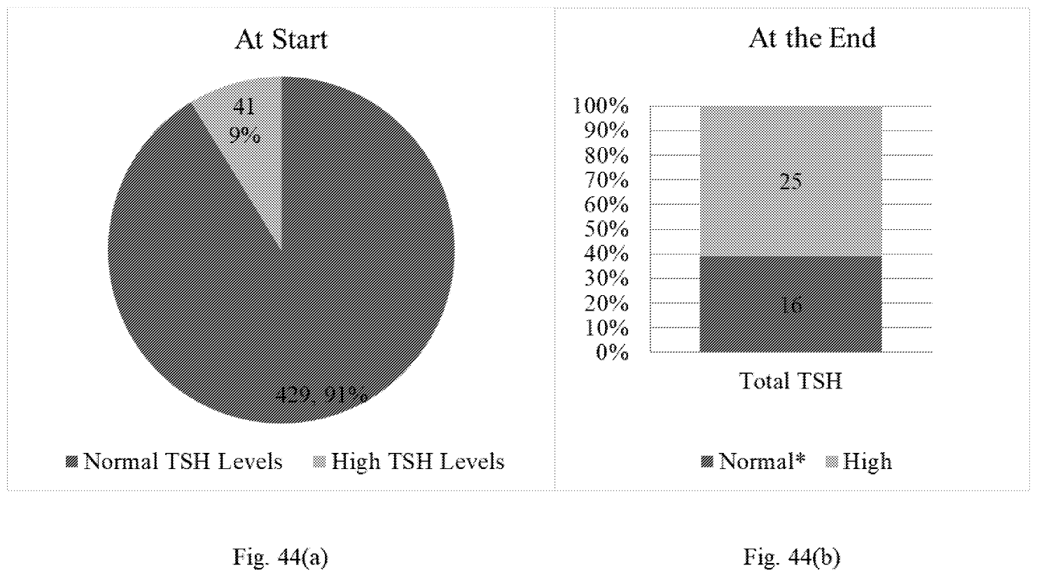

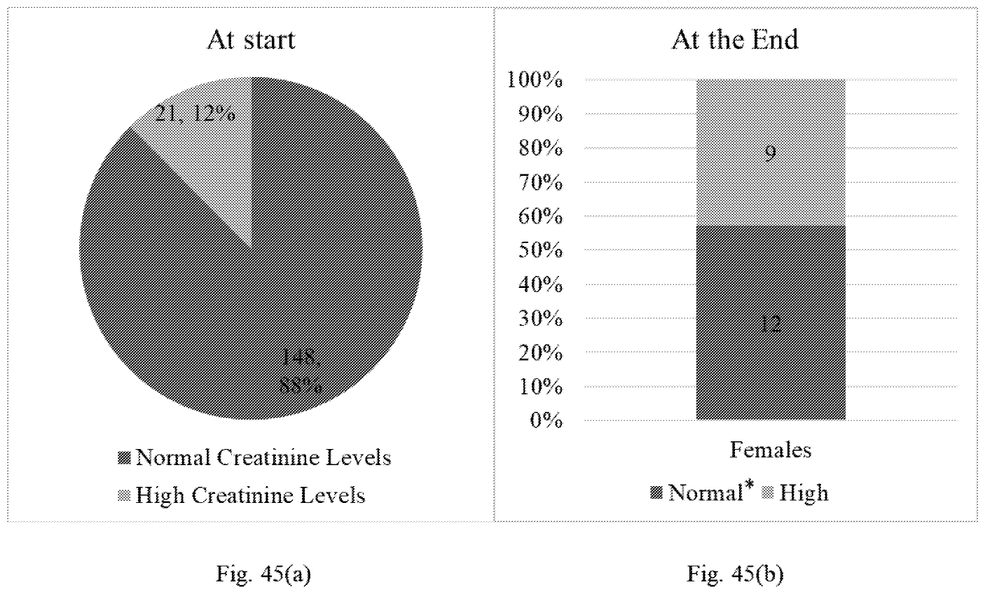

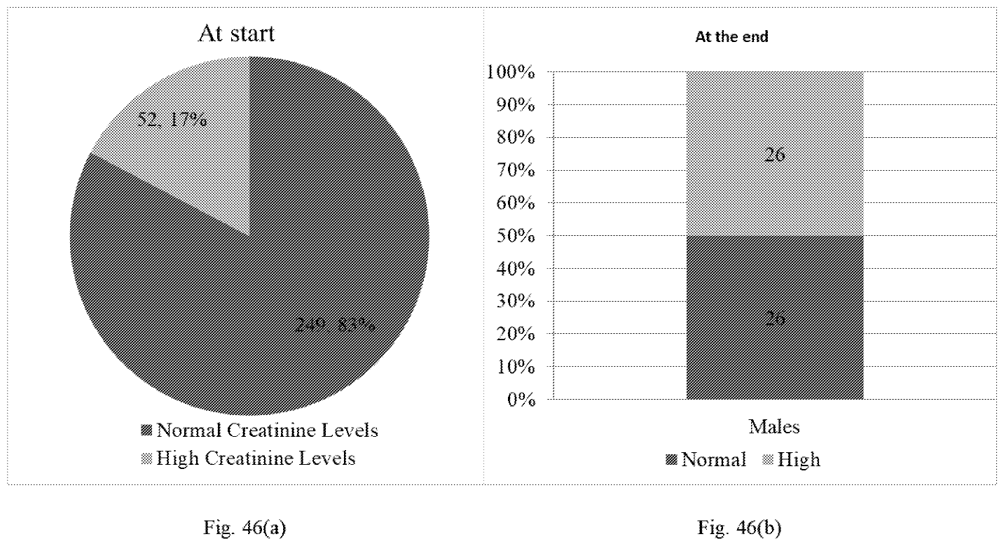

Group 5 consisted of 470 individuals with Type 2 diabetic/prediabetic status. FIG. 38 and Table 21 depict the age distribution among the participants. It was seen that with 19.9 percent, the age range of 40-50 years had the most number of individuals. Body Mass Index (BMI) of the individual participants was recorded both before and after the intervention program (FIG. 39). At the start of the program, nearly 41 percent of individuals had a BMI of between 25 and 30, corresponding to the ‘overweight’ category. At the end of the program, only 27 percent of individuals remained in this category. Similarly, the ‘obese’ category, at the start of the program, consisted of 17 percent of the 470 individuals, and at the end of the program, the category comprised of only 7 percent of individuals. The ‘healthy weight’ categories of BMI in the range of 21-25 had nearly 53 percent of the individuals, at the end of the program in contrast to only 33 percent at the start of the program. The absolute change in the weight is graphically depicted in FIG. 40, where it can be seen that individuals with a BMI of >30 on average lost 6.6 kilograms of weight after the intervention program. Individuals with BMI of 25-30 on average lost about 5.0 kilograms of weight, those with BMI 21-25 on average lost about 1.3 kilograms of weight, those with BMI<21 on average gained 0.6 kilograms of weight. FIG. 41 graphically represents the number of individuals at the end of program, who are no longer under medication. The percentage of individuals free of the need for medication can be seen in Table 22. Nearly 63 percentage of individuals no longer need insulin medication to manage diabetes, and 36 percent of individuals no longer need diabetes medication at the end of the program. A HbA1c test was conducted to see the difference in glycated hemoglobin levels at the start and the end of the intervention program, as depicted in FIG. 42. At the end of the program, it is seen that nearly 48 percent of all individuals showed<7 percent of HbA1c, which is considered good control for a diabetic subject. FIG. 43(a) is a pie chart of the cholesterol levels of individuals in Group 5 of the program at the start. FIG. 43(b) shows the distribution of individuals as a function of cholesterol levels at the end of the program. It is seen that of the 16 percent of individuals who started the program with high cholesterol levels, nearly 40 percent have normal levels of cholesterol at the end of the program. FIG. 44(a) is a pie chart of the Thyroid Stimulating Hormone (TSH) levels of individuals in Group 5 of the program at the start. FIG. 44(b) shows the distribution of individuals as a function of TSH levels at the end of the program. It is seen that of the 9 percent of individuals who started the program with high TSH levels, nearly 39 percent have normal levels of TSH at the end of the program. FIG. 45(a) is a pie chart of the creatinine levels of female individuals in Group 5 of the program at the start. FIG. 45(b) shows the distribution of female individuals of Group 5 as a function of creatinine levels at the end of the program. It is seen that of the 12 percent of individuals who started the program with high creatinine levels, nearly 56 percent had normal levels of creatinine at the end of the program. Similarly, FIG. 46(a) is a pie chart of the creatinine levels of male individuals in Group 5 of the program at the start. FIG. 46(b) shows the distribution of male individuals as a function of creatinine levels at the end of the program. It is seen that of the 17 percent of individuals who started the program with high creatinine levels, nearly 50 percent has normal levels of creatinine at the end of the program.

TABLE 21
Age Distribution in participants in Group 5

| Parameter | Number |
|---|---|
| Total no. of active participants | 470 |
| Primary diabetics | 394 |
| Weight loss | 34 |
| Supporting | 42 |

TABLE 22
Percentage of freedom from medication for participants in Group 5

| Parameter | Number |
|---|---|
| Insulin | 63.4 |
| Diabetes medication | 36 |
| Thyroid Medication | 0 |
| Hypertension Medication | 16.2 |
| Cholesterol Medication | 34.6 |

### Group 6

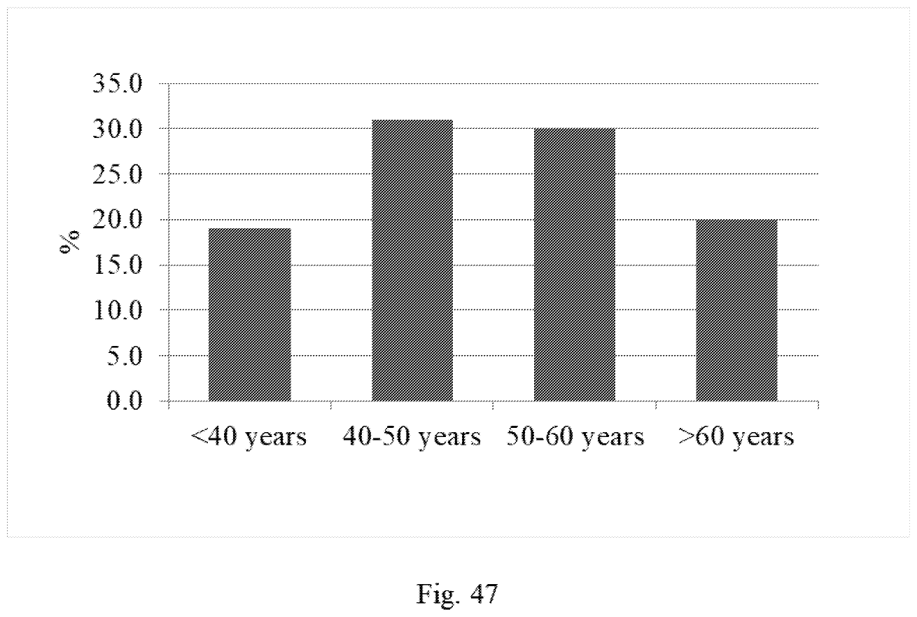

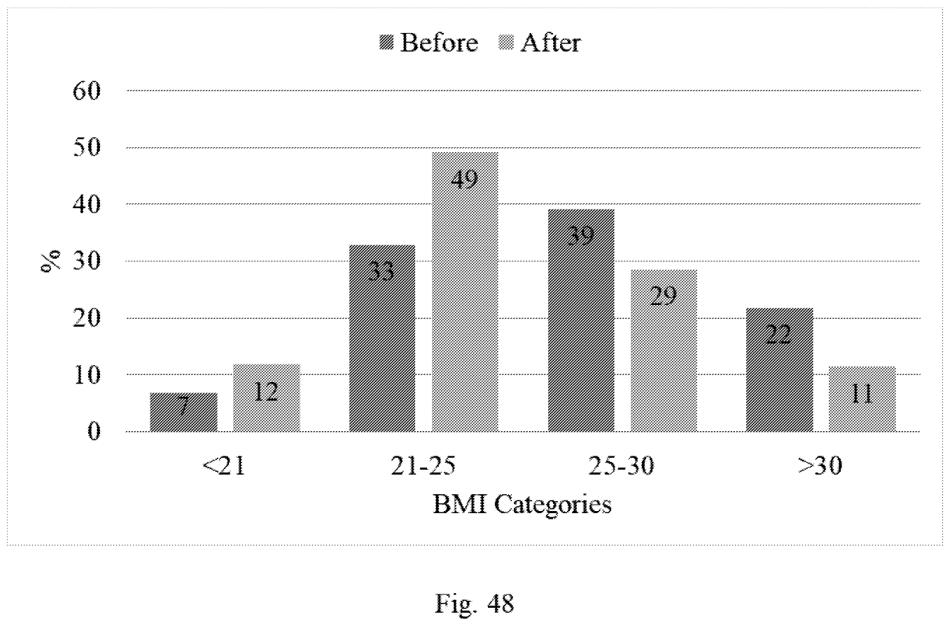

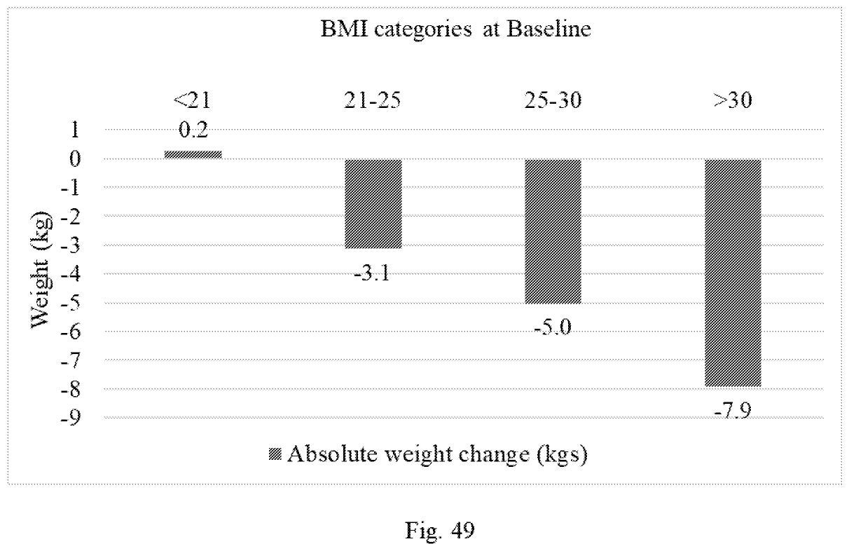

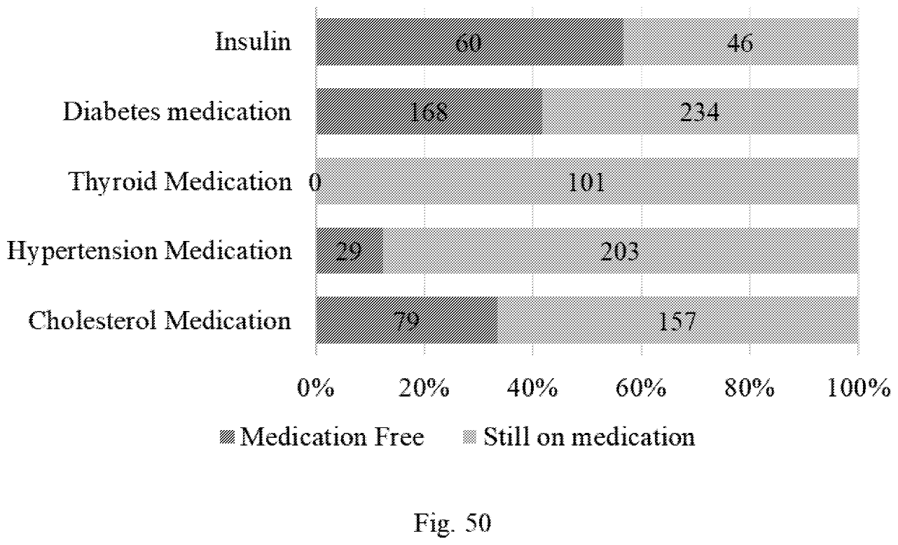

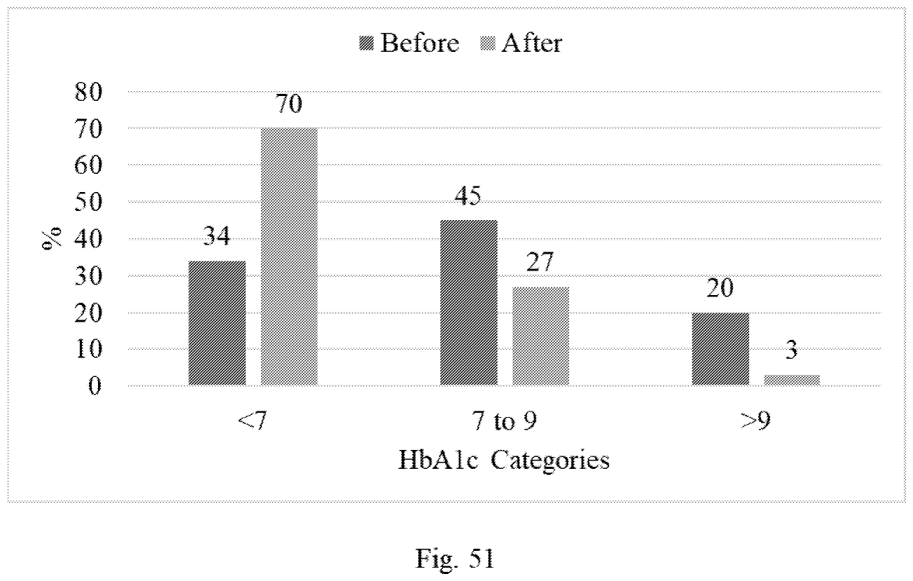

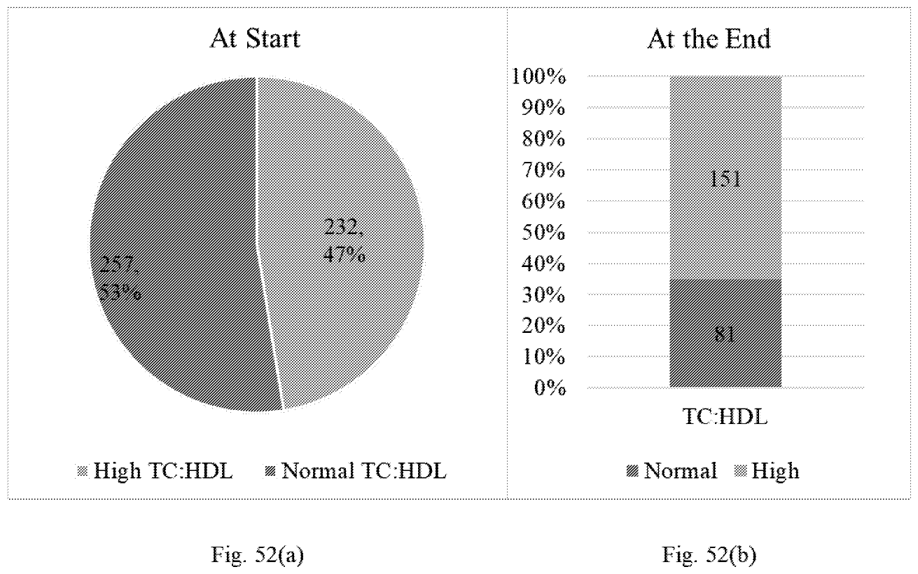

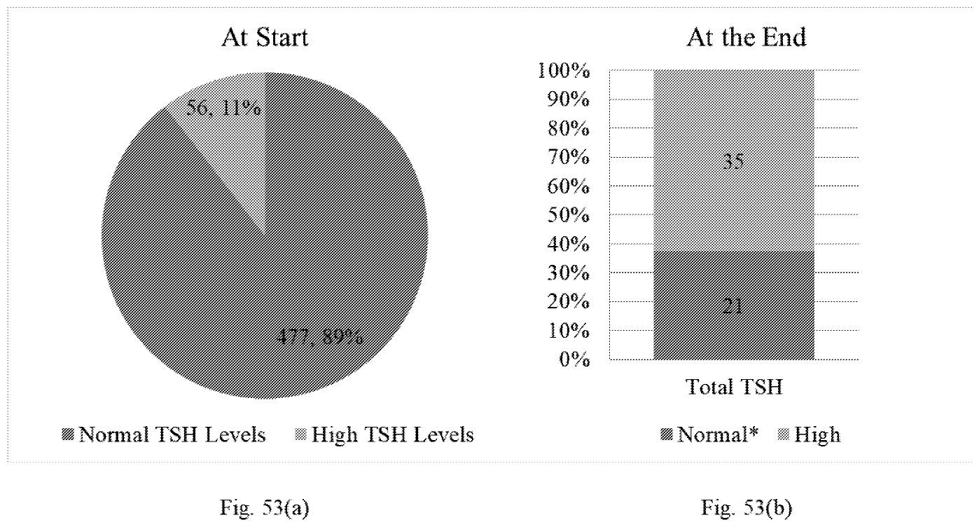

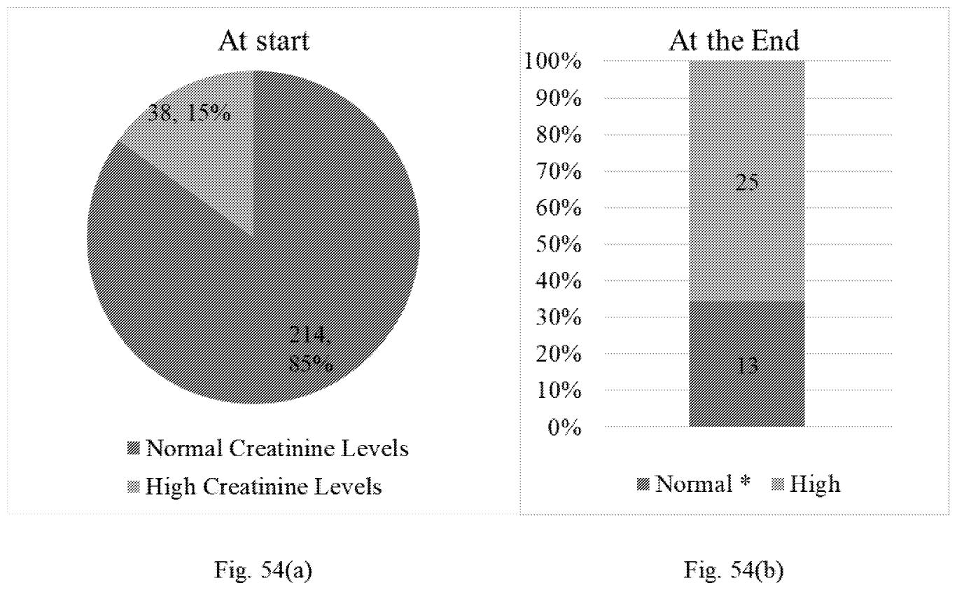

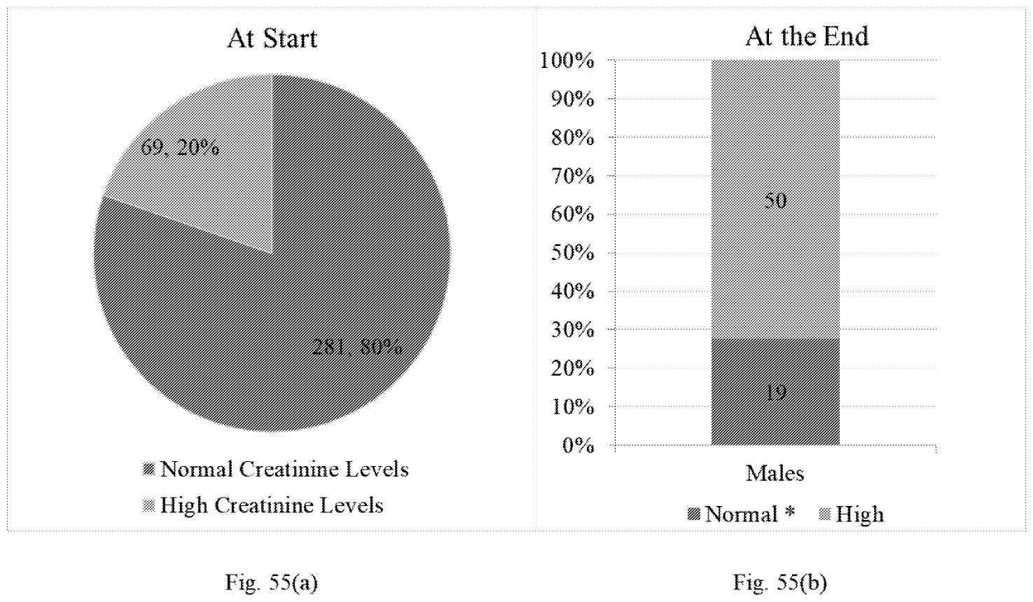

Group 6 consisted of 602 individuals with Type 2 diabetic/prediabetic status. FIG. 47 and Table 23 depict the age distribution among the participants. It was seen that with 31 percent, the age range of 40-50 years had the most number of individuals. Body Mass Index (BMI) of the individual participants was recorded both before and after the intervention program (FIG. 48). At the start of the program, nearly 39 percent of individuals had a BMI of between 25 and 30, 15 corresponding to the ‘overweight’ category. At the end of the program, only 29 percent of individuals remained in this category. Similarly, the ‘obese’ category, at the start of the program, consisted of 22 percent of the 602 individuals, and at the end of the program, the category comprised of only 11 percent of individuals. The ‘healthy weight’ categories of BMI in the range of 21-25 had nearly 49 percent of 20 the individuals, at the end of the program in contrast to only 33 percent at the start of the program. The absolute change in the weight is graphically depicted in FIG. 49, where it can be seen that individuals with a BMI of >30 on average, lost 7.9 kilograms of weight after the intervention program. Individuals with BMI of 25-30 on average, lost about 5.0 kilograms of weight, those with BMI 21-25 on average, lost about 3.1 kilograms of weight, those with BMI<21 on an average, gained 0.2 kilograms of weight. FIG. 50 graphically represents the number of individuals at the end of program, who are no longer under medication. The percentage of individuals free of the need for medication can be seen in Table 24. Nearly 57 percentage of individuals no longer need insulin medication to manage diabetes, and 37 percent of individuals no longer need diabetes medication at the end of the program. A HbA1c test was conducted to see the difference in glycated hemoglobin levels at the start and the end of the intervention program, as depicted in FIG. 51. At the end of the program, it is seen that nearly 70 percent of all individuals showed<7 percent of HbA1c, which is considered as good control for a diabetic subject. FIG. 52(a) is a pie chart of the cholesterol levels of individuals in Group 6 of the program at the start. FIG. 52(b) shows the distribution of individuals as a function of cholesterol levels at the end of the program. It is seen that of the 53 percent of individuals who started the program with high cholesterol levels, nearly 35 percent have normal levels of cholesterol at the end of the program. FIG. 53(a) is a pie chart of the Thyroid Stimulating Hormone (TSH) levels of individuals in Group 6 of the program at the start. FIG. 53(b) shows the distribution of individuals as a function of TSH levels at the end of the program. It is seen that of the 11 percent of individuals who started the program with high TSH levels, nearly 37 percent have normal levels of TSH at the end of the program. FIG. 54(a) is a pie chart of the creatinine levels of female individuals in Group 6 of the program at the start. FIG. 54(b) shows the distribution of female individuals of Group 6 as a function of creatinine levels at the end of the program. It is seen that of the 15 percent of individuals who started the program with high creatinine levels, nearly 35 percent had normal levels of creatinine at the end of the program. Similarly, FIG. 55(a) is a pie chart of the creatinine levels of male individuals in Group 6 of the program at the start. FIG. 55(b) shows the distribution of male individuals as a function of creatinine levels at the end of the program. It is seen that of the 20 percent of individuals who started the program with high creatinine levels, nearly 27 percent has normal levels of creatinine at the end of the program.

TABLE 23
Age Distribution in participants in Group 6

| Parameter | Number |
|---|---|
| Total no. of active participants | 602 |
| Primary diabetics | 484 |
| Weight loss | 82 |
| Supporting | 36 |

TABLE 24
Percentage of freedom from medication for participants in Group 6

| Parameter | Number |
|---|---|
| Insulin | 57 |
| Diabetes medication | 37 |
| Thyroid Medication | 0 |
| Hypertension Medication | 12 |
| Cholesterol Medication | 34 |

### Group 7

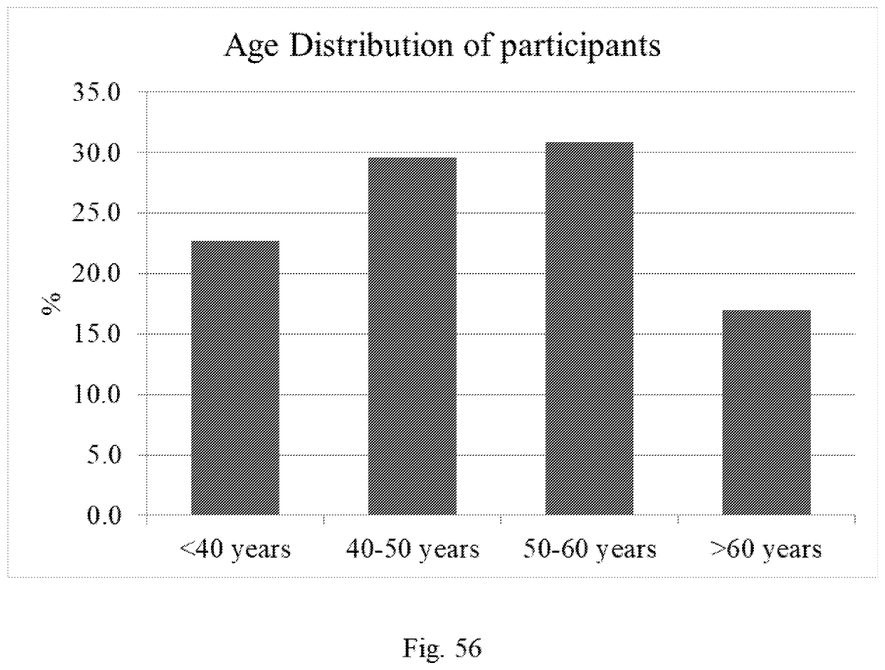

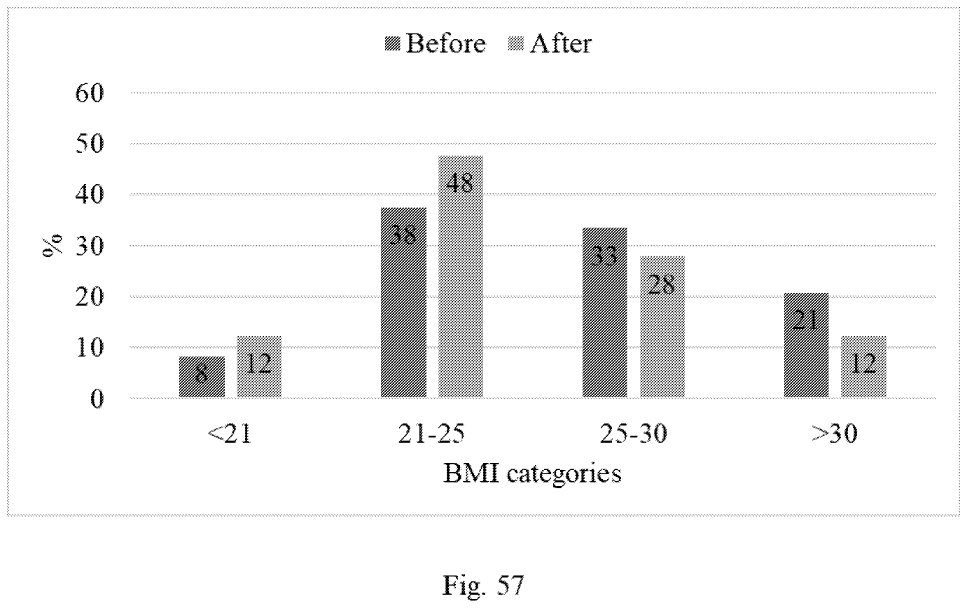

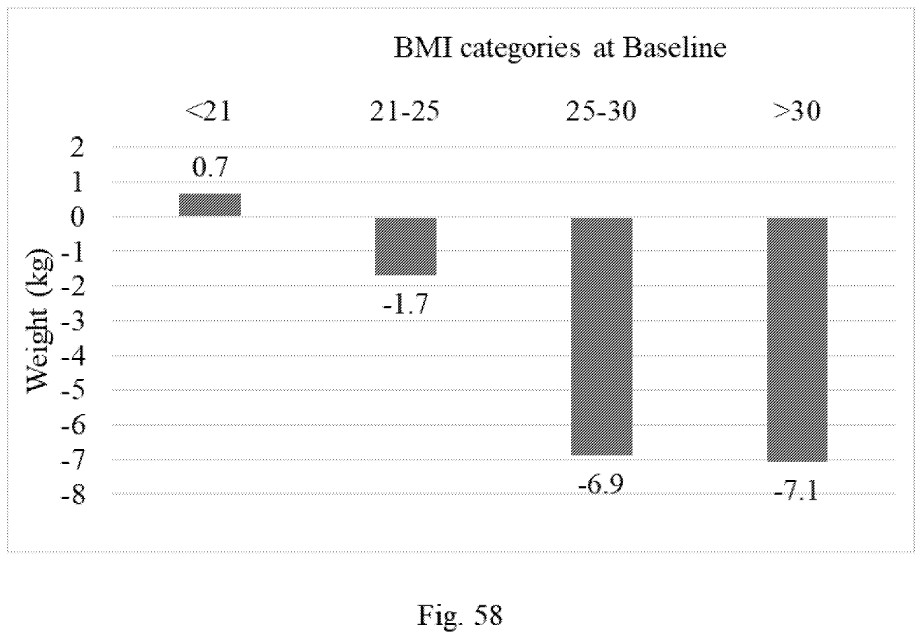

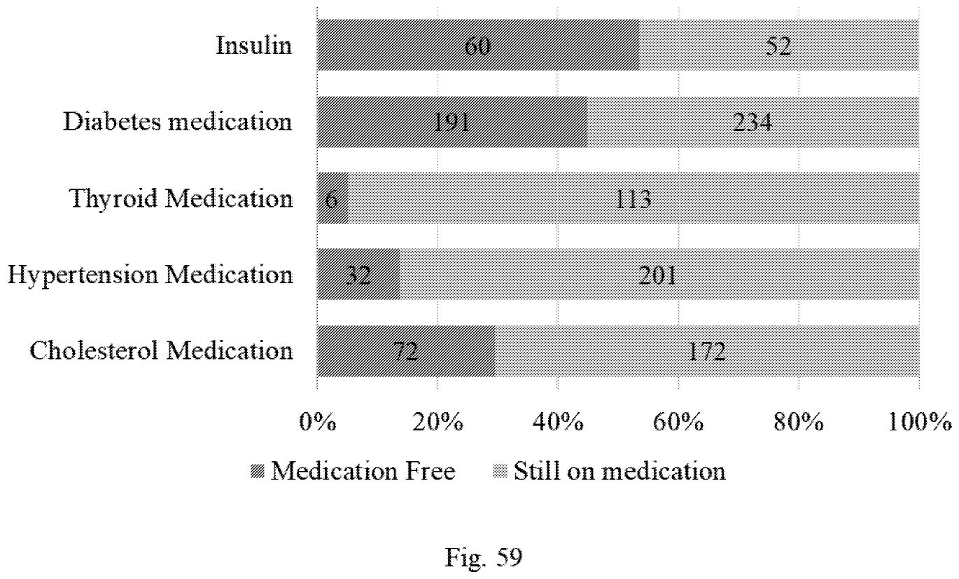

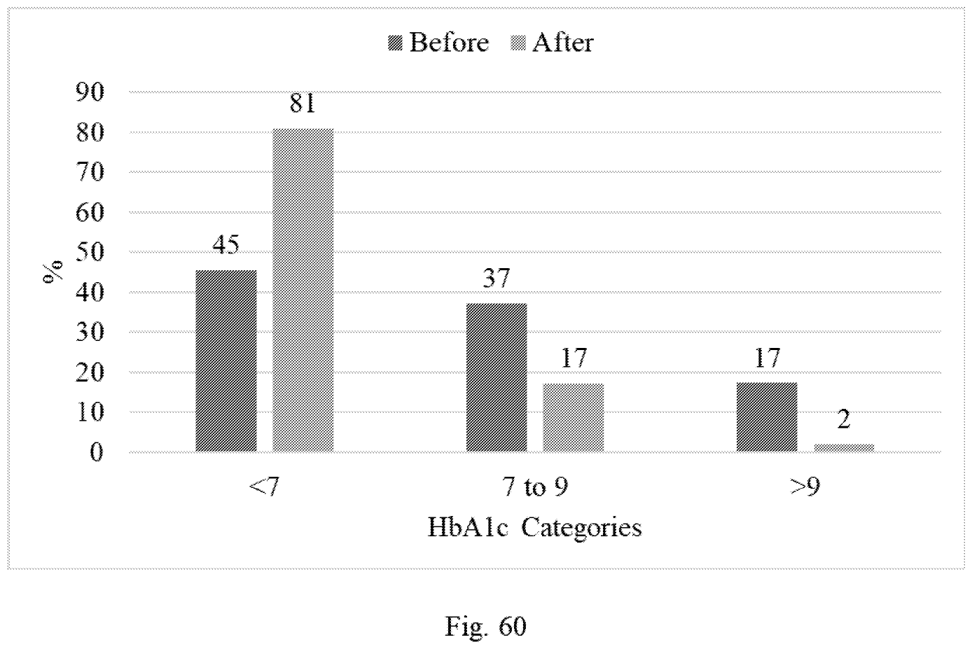

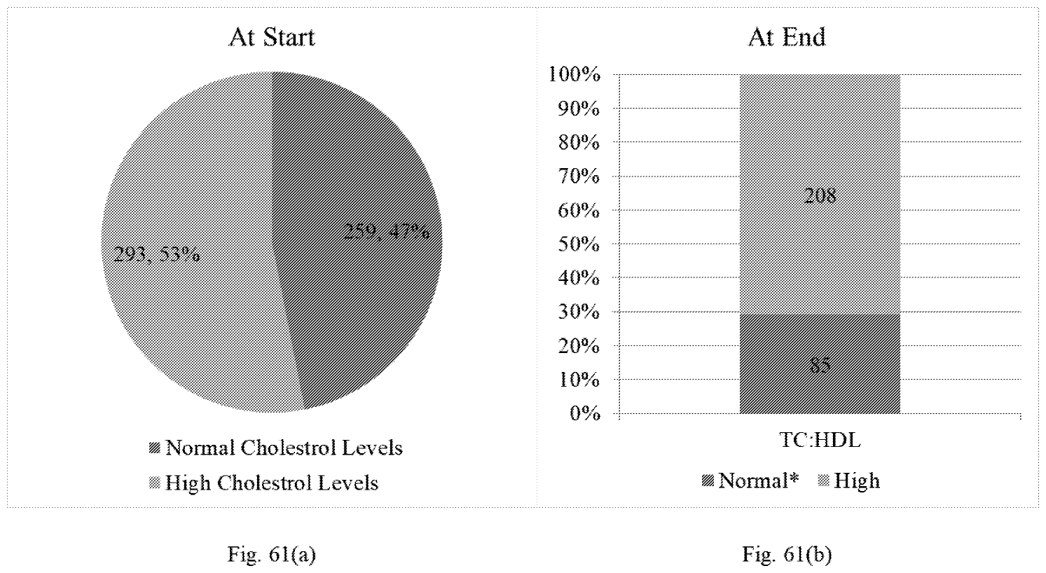

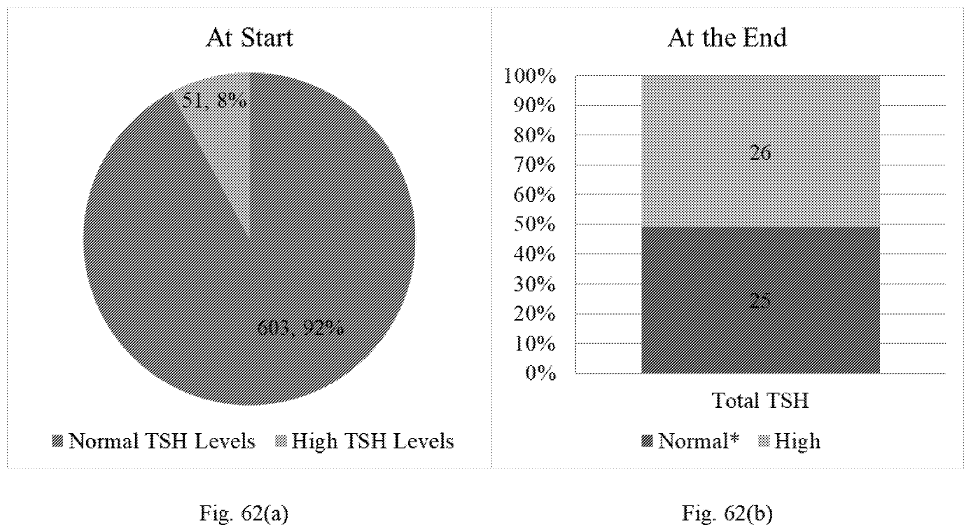

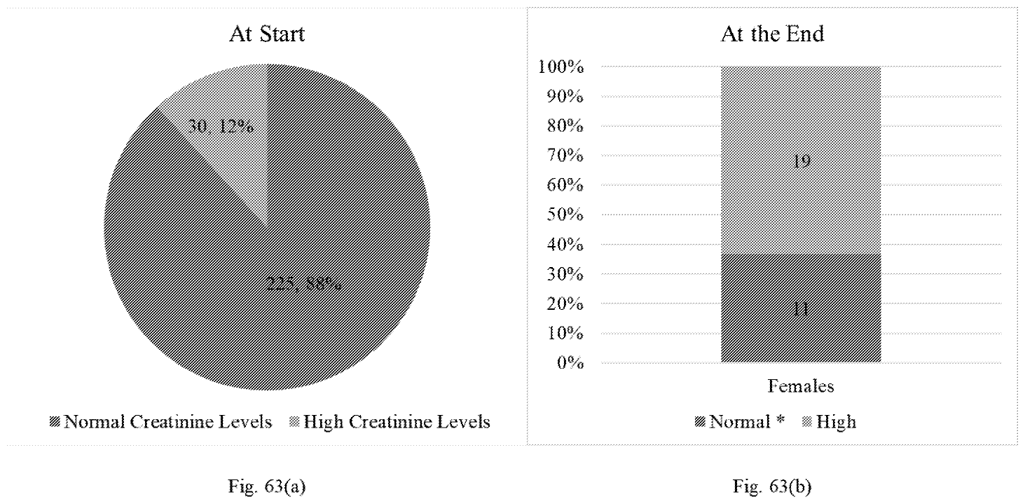

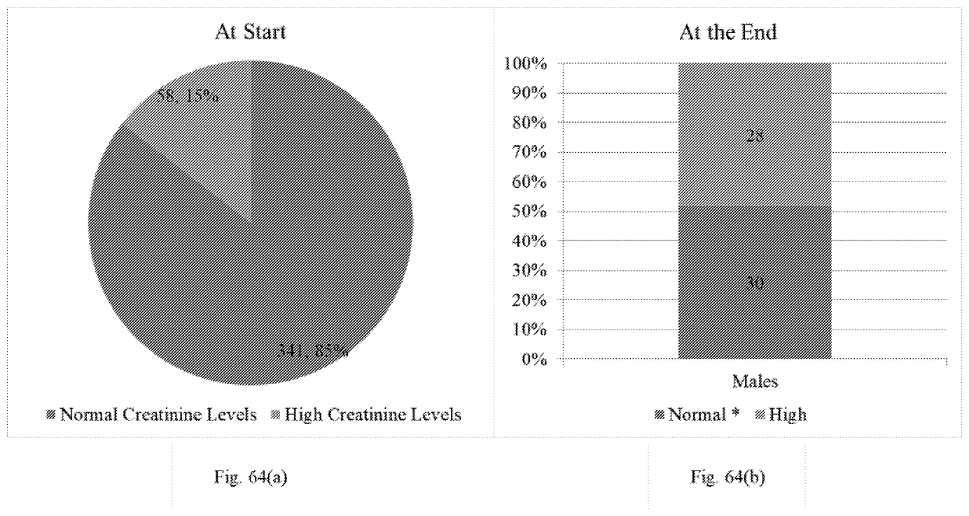

Group 7 consisted of 654 individuals with Type 2 diabetic/prediabetic status. FIG. 56 and Table 25 depict the age distribution among the participants. It was seen that with 30.9 percent, the age range of 50-60 years had the most number of individuals. Body Mass Index (BMI) of the individual participants was recorded both before and after the intervention program (FIG. 57). At the start of the program, nearly 33 percent of individuals had a BMI of between 25 and 30, corresponding to the ‘overweight’ category. At the end of the program, only 28 percent of individuals remained in this category. Similarly, the ‘obese’ category, at the start of the program, consisted of 21 percent of the 654 individuals, and at the end of the program, the category comprised of 12 percent of individuals. The ‘healthy weight’ categories of BMI in the range of 21-25 had nearly 48 percent 20 of the individuals, at the end of the program in contrast to only 38 percent at the start of the program. The absolute change in the weight is graphically depicted in FIG. 58, where it can be seen that individuals with a BMI of >30 on an average, lost 7.1 kilograms of weight after the intervention program. Individuals with BMI of 25-30, on average, lost about 6.9 kilograms of weight, those with BMI 21-25 on average lost about 1.7 kilograms of weight, those with BMI<21, on an average, gained 0.7 kilograms of weight. FIG. 59 graphically represents the number of individuals at the end of program, who are no longer under medication. The percentage of individuals free of the need for medication can be seen in Table 26. Nearly 54 percentage of individuals no longer need insulin medication to manage diabetes, and 39 percent of individuals no longer need diabetes medication at the end of the program. A HbA1c test was conducted to see the difference in glycated hemoglobin levels at the start and the end of the intervention program, as depicted in FIG. 60. At the end of the program, it is seen that nearly 81 percent of all individuals showed<7 percent of HbA1c, which is considered as good control for a diabetic subject. FIG. 61(a) is a pie chart of the cholesterol levels of individuals in Group 7 of the program at the start. FIG. 61(b) shows the distribution of individuals as a function of cholesterol levels at the end of the program. It is seen that of the 53 percent of individuals who started the program with high cholesterol levels, nearly 29 percent have normal levels of cholesterol at the end of the program. FIG. 62(a) is a pie chart of the Thyroid Stimulating Hormone (TSH) levels of individuals in Group 7 of the program at the start. FIG. 62(b) shows the distribution of individuals as a function of TSH levels at the end of the program. It is seen that of the 8 percent of individuals who started the program with high TSH levels, nearly 49 percent have normal levels of TSH at the end of the program. FIG. 63(a) is a pie chart of the creatinine levels of female individuals in Group 7 of the program at the start. FIG. 63(b) shows the distribution of female individuals of Group 7 as a function of creatinine levels at the end of the program. It is seen that of the 12 percent of individuals who started the program with high creatinine levels, nearly 36 percent had normal levels of creatinine at the end of the program. Similarly, FIG. 64(a) is a pie chart of the creatinine levels of male individuals in Group 7 of the program at the start. FIG. 64(b) shows the distribution of male individuals as a function of creatinine levels at the end of the program. It is seen that of the 15 percent of individuals who started the program with high creatinine levels, nearly 50 percent has normal levels of creatinine at the end of the program.

TABLE 25
Age Distribution in participants in Group 7

| Parameter | Number |
|---|---|
| Total no. of active participants | 654 |
| Primary diabetics | 526 |
| Weight loss | 81 |
| Supporting | 47 |

TABLE 26
Percentage of freedom from medication for participants in Group 7

| Parameter | Number |
|---|---|
| Insulin | 54 |
| Diabetes medication | 39 |
| Thyroid Medication | 5 |
| Hypertension Medication | 14 |
| Cholesterol Medication | 30 |

### Results

A total number of 3926 Type 2 diabetic/prediabetic individuals partook in the controlled intervention program. The 7 groups underwent the 5 phases of the 12 month program. Distribution of BMI and absolute weight change graphs for all groups indicate that the intervention program helped participants in weight loss/management. Participants also showed a significant reduction in blood glucose levels. The need for medication such as insulin, OHA, thyroid medication, or cholesterol lowering drugs also significantly decreased throughout individuals in the 7 groups. Further, creatinine levels, cholesterol levels, and TSH levels are seen to return nearing normal levels following the program. The experimental study validates that obesity and obesity related disorders such as diabetes, hypertension, hypocholesteremia, and hyper/hypothyroidism can be prevented or managed by following the controlled 12 month intervention program.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

We claim:

1. A system (100) for monitoring and management of lifestyle of a subject, said system (100) comprising:
at least one input device (102) associated with the subject, said input device (102) is configured to:
capture input data of the subject, wherein said input data is received by subjecting the subject to,
an alkalizing diet, an exercise protocol comprising circulation and activation exercises, and determining improvement in subject's health condition;
a cleansing diet, or SNS meal plan for subject requiring additional weight loss, an exercise protocol comprising yoga and strengthening exercises, and determining improvement in subject's health condition;
the cleansing diet or a muscle building diet, an exercise protocol comprising of yoga and strengthening exercises or sampling and athletic specialization exercises, a meditation protocol for release of stress, and determining improvement in subject's health condition;
the muscle building diet, an exercise protocol comprising sampling and specialization exercises, and determining improvement in subject's health condition; and
a specific diet and exercise chart to sustain reversal and phase 4 athletic identity; and
a subject monitoring device (108) coupled to the at least one input device (102), said subject monitoring device (108) is configured to:
generate results for the subject by processing the input data of the subject captured by the at least one input device (102);
process and monitor the generated results of the subject based on the input data in a closed loop control operation for management of lifestyle, of the subject; and
provide the results of the subject to at least one of, the subject, a healthcare professional, and a doctor.

2. The system (100) as claimed in claim 1, wherein the subject monitoring device (108) is further configured to:
provide a prognosis to the subject by evaluating differences between the input data and the results of the subject; and
provide a diet and exercise protocol to the subject, based on the input data of the subject.

3. The system (100) as claimed in claim 1, wherein each of said diet is a vegan diet which is free of milk and milk product, wherein said vegan diet comprises,
a green smoothie comprising at least one green leafy vegetable selected from a group consisting of spinach, kale, lettuce and green sorrel;
at least one fruit selected from a group consisting of apple, pear, guava, banana and papaya;
leaves of at least two herbs selected from a group consisting of Murraya koenigii, Piper betle, Ocimum tenuiflorum, basil, and mint leaves;
a protein rich breakfast comprising sprouts at a portion size of 25%, salads at a portion size of 25% and cooked Lentil-based food at a portion size of 50%, of said breakfast; and
a principle meal comprising 25% of each of raw salad, cooked vegetable, grain and lentil-based food.

4. The system (100) as claimed in claim 1, wherein said alkalizing diet comprises anti-diabetogenic food, wherein calorie intake of said diet is in the range of 1300 to 1400.

5. The system (100) as claimed in claim 1, wherein said cleansing diet comprises iron and vitamin rich food; intermittent or water fasting for a duration of half a day or one day;
and at least one portion of juice.

6. The system (100) as claimed in claim 5, wherein said juice is selected from, red juice comprising suitable quantities of tomato, carrot, red capsicum and yellow capsicum;
green juice comprising suitable quantities of ash gourd, apple, capsicum, ridge gourd and lemon juice, and white juice, and/or suitable quantities of bottle gourd, green sorrel/Chenopodium, coriander leaves and lemon juice; and
white juice comprising suitable quantities of bottle gourd, cucumber, and lemon juice.

7. The system (100) as claimed in claim 1, wherein said SNS meal plan is a meal replacement plan wherein the subject is provided with 2 glasses of green smoothie in the morning, 25 to 40 nuts for lunch, and 400 to 500 ml soup for dinner.

8. The system (100) as claimed in claim 1, wherein said muscle building diet comprises muscle building food, wherein calorie intake of said diet is in the range of 1800 to 2200 and the ratio of Carbohydrates:Protein:Fat is in the range of 4 to 5:1 to 1.25:1 to 1.25.

9. The system (100) as claimed in claim 1, wherein said portion size of said diet is based on body type of said subject, wherein said body type is selected from Ectomorph, Thin mesomorph, Obese Mesomorph and Endomorph.

10. The system (100) as claimed in claim 1, said circulation and activation exercises comprise exercises selected from a group consisting of warm up, super brain yoga, palm plank, push-ups and anti-gravity exercises comprising stairway ascending and descending exercise and nitric oxide dump, which is based on BMI of lesser than 25 or greater than 25.

11. The system (100) as claimed in claim 1, wherein sampling and specialization exercises comprise strength building exercise, yoga, and cardio exercise at a ratio of 3:2:1, and a specialization in a specific form of exercise.

12. The system (100) as claimed in claim 1, wherein the cleansing diet further comprises a portion of Shahi pani as a hunger suppressant, comprising water, sabja/chia seeds, rock salt, kesar and lemon juice.

13. The system (100) as claimed in claim 1, wherein said improvement is determined in one or more group sessions with said subject and one or more healthcare professional or doctor for said subject, and with or without other participants.

14. The system (100) as claimed in claim 12, wherein said improvement is determined based on a change in said subject's requirement of diabetes medication, fastingblood glucose level (mg/dl), HbA1C level (%), BMI (kg/m²) and waist circumference (cm).

15. A method for monitoring and management of lifestyle of a subject, said method comprising:
capturing, by at least one input device (102) associated with the subject, at least one input data of the subject;
generating, by a subject monitoring device (108), results for the subject by processing the input data of the subject captured by the at least one input device (102);
processing and monitoring, by the subject monitoring device (108), the generated results of the subject based on the input data in a closed loop control operation for management of lifestyle, of the subject; and
providing, by the subject monitoring device (108), the results of the subject to at least one of, the subject, a healthcare professional, and a doctor.

16. The method as claimed in claim 15, wherein said capturing, by at least one input device (102) associated with the subject, at least one input data of the subject includes,
receiving by the by at least one input device (102) associated with the subject, the at least one input data by subjecting the subject to,
an alkalizing diet, an exercise protocol comprising circulation and activation exercises, and determining improvement in subject's health condition;
a cleansing diet, or SNS meal plan for subject requiring additional weight loss, an exercise protocol comprising yoga and strengthening exercises, and determining improvement in subject's health condition;
the cleansing diet or a muscle building diet, an exercise protocol comprising of yoga and strengthening exercises or sampling and athletic specialization exercises, a meditation protocol for release of stress, and determining improvement in subject's health condition;
the muscle building diet, an exercise protocol comprising sampling and specialization exercises, and determining improvement in subject's health condition; and
a specific diet and exercise chart to sustain reversal and phase 4 athletic identity,
wherein
each of said diet is a vegan diet which is free of milk and milk product, wherein said vegan diet comprises,
a green smoothie comprising at least one green leafy vegetable selected from a group consisting of spinach, kale, lettuce and green sorrel;
at least one fruit selected from a group consisting of apple, pear, guava, banana and papaya;
leaves of at least two herbs selected from a group consisting of Murraya koenigii, Piper betle, Ocimum tenuiflorum, basil, and mint leaves;
a protein rich breakfast comprising sprouts at a portion size of 25%, salads at a portion size of 25% and cooked Lentil-based food at a portion size of 50%, of said breakfast; and
a principle meal comprising 25% of each of raw salad, cooked vegetable, grain and lentil-based food.

17. The method as claimed in claim 15, wherein said method includes,
providing, by the subject monitoring device (108), a prognosis to the subject by evaluating differences between the input data and the results of the subject; and
providing, by the subject monitoring device (108), a diet and exercise protocol to the subject, based on the input data of the subject.

18. The method as claimed in claim 15, wherein said alkalizing diet comprises anti-diabetogenic food, wherein calorie intake of said diet is in the range of 1300 to 1400.

19. The method as claimed in claim 15, wherein said cleansing diet comprises iron and vitamin rich food; intermittent or water fasting for a duration of half a day or one day;
and at least one portion of juice.

20. The method as claimed in claim 19, wherein said juice is selected from red juice comprising suitable quantities of tomato, carrot, red capsicum and yellow capsicum;
green juice comprising suitable quantities of ash gourd, apple, capsicum, ridge gourd and lemon juice; and
white juice, and/or suitable quantities of bottle gourd, green sorrel/Chenopodium, coriander leaves and lemon juice; and white juice comprising suitable quantities of bottle gourd, cucumber, and lemon juice.

21. The method as claimed in claim 15, wherein said SNS meal plan is a meal replacement plan wherein the subject is provided with 2 glasses of green smoothie in the morning, 25 to 40 nuts for lunch, and 400 to 500 ml soup for dinner.

22. The method as claimed in claim 15, wherein said muscle building diet comprises muscle building food, wherein calorie intake of said diet is in the range of 1800 to 2200 and the ratio of Carbohydrates:Protein:Fat is in the range of 4 to 5:1 to 1.25:1 to 1.25.

23. The method as claimed in claim 15, wherein said portion size of said diet is based on body type of said subject, wherein said body type is selected from Ectomorph, Thin mesomorph, Obese Mesomorph and Endomorph.

24. The method as claimed in claim 15, said circulation and activation exercises comprises exercises selected from a group consisting of warm up, super brain yoga, palm plank, push-ups and anti-gravity exercises comprising stairway ascending and descending exercise and nitric oxide dump, which is based on BMI of lesser than 25 or greater than 25.

25. The method as claimed in claim 15, wherein sampling and specialization exercises comprises of strength building exercise, yoga, and cardio exercise at a ratio of 3:2:1, and a specialization in a specific form of exercise.

26. The method as claimed in claim 17, wherein the cleansing diet further comprises a portion of Shahi pani as a hunger suppressant, comprising water, sabja/chia seeds, rock salt, kesar and lemon juice.

27. The method as claimed in claim 15, wherein said improvement is determined in one or more group sessions with said subject and one or more healthcare professional or doctor of said subject, and with or without other participants.

28. The method as claimed in claim 15, wherein said improvement is determined based on a change in said subject's requirement of diabetes medication, fastingblood glucose level (mg/dl), HbA1C level (%), BMI (kg/m²) and waist circumference (cm).

29. A method for prevention, treatment, management, and/or reversal of Diabetes and related lifestyle disorders in a subject, said method comprising subjecting said subject to an alkalizing diet, an exercise protocol comprising circulation and activation exercises, and determining improvement in subject's condition;
a cleansing diet, or SNS meal plan for subject requiring additional weight loss, an exercise protocol comprising yoga and strengthening exercises, and determining improvement in subject's condition;
a cleansing diet or a muscle building diet, an exercise protocol comprising of yoga and strengthening exercises or sampling and athletic specialization exercises, a meditation protocol for release of stress, and determining improvement in subject's condition;
a muscle building diet, an exercise protocol comprising sampling and specialization exercises; and determining improvement in subject's condition; and
a specific diet and exercise chart to sustain reversal and phase 4 athletic identity,
wherein each of said diet is a vegan diet which is free of milk and milk product, wherein said vegan diet comprises
a green smoothie comprising at least one green leafy vegetable selected from a group consisting of spinach, kale, lettuce and green sorrel; at least one fruit selected from a group consisting of apple, pear, guava, banana and papaya; leaves of at least two herbs selected from a group consisting of Murraya koenigii, Piper betle, Ocimum tenuiflorum, basil, and mint leaves; protein rich breakfast comprising sprouts at a portion size of 25%, salads at a portion size of 25% and cooked Lentil-based food at a portion size of 50%, of said breakfast; and a principle meal comprising 25% of each of raw salad, cooked vegetable, grain and lentil-based food.

30. The method as claimed in claim 29, wherein said alkalizing diet is rich in anti-diabetogenic food, wherein calorie intake of said diet is in the range of 1300 to 1400.

31. The method as claimed in claim 29, wherein said cleansing diet comprises iron and vitamin rich food; intermittent or water fasting for a duration of half a day or one day;
and at least one portion of juice.

32. The method as claimed in claim 29, wherein said juice is selected from red juice comprising suitable quantities of tomato, carrot, red capsicum and yellow capsicum;
green juice comprising suitable quantities of ash gourd, apple, capsicum, ridge gourd and lemon juice, and white juice, and/or suitable quantities of bottle gourd, green sorrel/Chenopodium, coriander leaves and lemon juice; and
white juice comprising suitable quantities of bottle gourd, cucumber, and lemon juice.

33. The method as claimed in claim 29, wherein said SNS meal plan is a meal replacement plan wherein the subject is provided with 2 glasses of green smoothie in the morning, 25 to 40 nuts for lunch, and 400 to 500 ml soup for dinner.

34. The method as claimed in claim 29, wherein said muscle building diet is rich in muscle building food, wherein calorie intake of said diet is in the range of 1800 to 2200 and the ratio of Carbohydrates:Protein:Fat is in the range of 4 to 5:1 to 1.25:1 to 1.25.

35. The method as claimed in claim 29, wherein said portion size of said diet is based on body type of said subject, wherein said body type is selected from Ectomorph, Thin mesomorph, Obese Mesomorph and Endomorph.

36. The method as claimed in claim 29, said circulation and activation exercises comprises exercises selected from a group consisting of warm up, super brain yoga, palm plank, push-ups and anti-gravity exercises comprising stairway ascending and descending exercise and nitric oxide dump, which is based on BMI of lesser than 25 or greater than 25.

37. The method as claimed in claim 29, wherein sampling and specialization exercises comprises of strength building exercise, yoga, and cardio exercise at a ratio of 3:2:1, and a specialization in a specific form of exercise.

38. The method as claimed in claim 29, wherein the cleansing diet further comprises a portion of Shahi pani as a hunger suppressant, comprising water, sabja/chia seeds, rock salt, kesar and lemon juice.

39. The method as claimed in claim 29, wherein said improvement is determined in one or more group sessions with said subject and one or more healthcare professional or doctor for said subject, and with or without other participants.

40. The method as claimed in claim 29, wherein said improvement is determined based on a change in said subject's requirement of diabetes medication, fasting blood glucose level (mg/dl), HbA1C level (%), BMI (kg/m²) and waist circumference (cm).
